SEQUENCE LISTING (111 sequences)

SEQ ID NO: 1 - DNA, 1586 bp, Homo sapiens
cagagtaaccccgctctcgtgacctttcccctccattccgcacctccgagtgctggccgggcgagaggctggcggctgggctctcgcgcccctccctgcagggctcaggctctccccctcctgtcttctccgcgctgttcctcgtcatggcggccctcagcaagtccatccctcataactgctatgagatcggccacacttggcacccttcctgccgggtctccttcctgcagatcaccgggggcgccctggaggagtccctgaagatctatgctcctctgtacttgattgcagcaattctccggaaacggaaattagactattatttacacaaactactccctgagatcctacaatccgcttcatttctaactgctaatggggccttgtatatggctttcttttgcattttaaggaagatacttggaaaattctactcatggactcctggctttggtgccgctctgccagcatcttatgtggccattctcattgaaagaaaaagcaggagagggctgctcacaatttatatggccaacttggccacagaaacactattcagaatgggtgtagcaagaggaaccatcacaacattaagaaatggagaagtccttttgttttgcatcacagctgccatgtacatgttctttttcaggtgcaaggatggcttgaaaggatttacattttctgcacttaggttcattgtagggaaggaagaaattcccacacattctttttcaccagaggcagcatatgcaaaagtggaacaaaagagagagcaacatgaggaaaaacccagaagaatgaatatgattggtctagtcaggaaatttgtggattcaatatgcaaacatggaccaaggcatagatgttgcaaacattatgaagataattgcatctcttattgcattaaaggtttcatcagaatgtttagcgtggggtacttgatccagtgctgcctccgaatcccttctgcatttaggcatctgtttacacagccatctcggctactttctctcttctacaataaagaaaacttccagcttggagcttttcttggctcttttgttagtatatacaagggtactagttgcttcctgcgctggatcagaaacttagatgatgaactacatgctattatagctggatttttggcaggtatatcaatgatgttttataaaagcacaacaatttccatgtatttagcgtccaaattggtagagacaatgtatttcaaaggcattgaagcagggaaggttccctattttcctcatgcagatactatcatctattccatctctacagcaatttgcttccaggcagctgtcatggaagttcagactttgagaccatcttactggaagttccttttaagactcaccaagggcaaatttgctgtcatgaaccgaaaagtccttgatgtttttggtactgctgcatctaaacactttcaggatttcatccccaggttggatccaagatacacaactgtaacaccagagttgcccacagagttttcctgaagatgactgtaacttattaatgtgactaaatgtttcatcttgaagagttaattatgttgaaca

SEQ ID NO: 2 - protein, 458 aa, Homo sapiens [sequence omitted]

SEQ ID NO: 3 - DNA, 9497 bp, Homo sapiens
  misc_feature (1)..(9497): n = a,c,g, or t
caaacatgtcagctgttactggaagtggcctggcctctatttatcttcctgatcctgatctctgttcggctgagctacccaccctatgaacaacatgaatgccattttccaaataaagccatgccctctgcaggaacacttccttgggttcaggggattatctgtaatgccaacaacccctgtttccgttacccgactcctggggaggctcccggagttgttggaaactttaacaaatccattgtggctcgcctgttctcagatgctcggaggcttcttttatacagccagaaagacaccagcatgaaggacatgcgcaaagttctgagaacattacagcagatcaagaaatccagctcaaacttgaagcttcaagatttcctggtggacaatgaaaccttctctgggttcctgtatcacaacctctctctcccaaagtctactgtggacaagatgctgagggctgatgtcattctccacaaggtatttttgcaaggctaccagttacatttgacaagtctgtgcaatggatcaaaatcagaagagatgattcaacttggtgaccaagaagtttctgagctttgtggcctaccaagggagaaactggctgcagcagagcgagtacttcgttccaacatggacatcctgaagccaatcctgagaacactaaactctacatctcccttcccgagcaaggagctggccgaagccacaaaaacattgctgcatagtcttgggactctggcccaggagctgttcagcatgagaagctggagtgacatgcgacaggaggtgatgtttctgaccaatgtgaacagctccagctcctccacccaaatctaccaggctgtgtctcgtattgtctgcgggcatcccgagggaggggggctgaagatcaagtctctcaactggtatgaggacaacaactacaaagccctctttggaggcaatggcactgaggaagatgctgaaaccttctatgacaactctacaactccttactgcaatgatttgatgaagaatttggagtctagtcctctttcccgcattatctggaaagctctgaagccgctgctcgttgggaagatcctgtatacacctgacactccagccacaaggcaggtcatggctgaggtgaacaagaccttccaggaactggctgtgttccatgatctggaaggcatgtgggaggaactcagccccaagatctggaccttcatggagaacagccaagaaatggaccttgtccggatgctgttggacagcagggacaatgaccacttttgggaacagcagttggatggcttagattggacagcccaagacatcgtggcgtttttggccaagcacccagaggatgtccagtccagtaatggttctgtgtacacctggagagaagctttcaacgagactaaccaggcaatccggaccatatctcgcttcatggagtgtgtcaacctgaacaagctagaacccatagcaacagaagtctggctcatcaacaagtccatggagctgctggatgagaggaagttctgggctggtattgtgttcactggaattactccaggcagcattgagctgccccatcatgtcaagtacaagatccgaatggacattgacaatgtggagaggacaaataaaatcaaggatgggtactgggaccctggtcctcgagctgacccctttgaggacatgcggtacgtctgggggggcttcgcctacttgcaggatgtggtggagcaggcaatcatcagggtgctgacgggcaccgagaagaaaactggtgtctatatgcaacagatgccctatccctgttacgttgatgacatctttctgcgggtgatgagccggtcaatgcccctcttcatgacgctggcctggatttactcagtggctgtgatcatcaagggcatcgtgtatgagaaggaggcacggctgaaagagaccatgcggatcatgggcctggacaacagcatcctctggtttagctggttcattagtagcctcattcctcttcttgtgagcgctggcctgctagtggtcatcctgaagttaggaaacctgctgccctacagtgatcccagcgtggtgtttgtcttcctgtccgtgtttgctgtggtgacaatcctgcagtgcttcctgattagcacactcttctccagagccaacctggcagcagcctgtgggggcatcatctacttcacgctgtacctgccctacgtcctgtgtgtggcatggcaggactacgtgggcttcacactcaagatcttcgctagcctgctgtctcctgtggcttttgggtttggctgtgagtactttgccctttttgaggagcagggcattggagtgcagtgggacaacctgtttgagagtcctgtggaggaagatggcttcaatctcaccacttcggtctccatgatgctgtttgacaccttcctctatggggtgatgacctggtacattgaggctgtctttccaggccagtacggaattcccaggccctggtattttccttgcaccaagtcctactggtttggcgaggaaagtgatgagaagagccaccctggttccaaccagaagagaatatcagaaatctgcatggaggaggaacccacccacttgaagctgggcgtgtccattcagaacctggtaaaagtctaccgagatgggatgaaggtggctgtcgatggcctggcactgaatttttatgagggccagatcacctccttcctgggccacaatggagcggggaagacgaccaccatgtcaatcctgaccgggttgttccccccgacctcgggcaccgcctacatcctgggaaaagacattcgctctgagatgagcaccatccggcagaacctgggggtctgtccccagcataacgtgctgtttgacatgctgactgtcgaagaacacatctggttctatgcccgcttgaaagggctctctgagaagcacgtgaaggcggagatggagcagatggccctggatgttggtttgccatcaagcaagctgaaaagcaaaacaagccagctgtcaggtggaatgcagagaaagctatctgtggccttggcctttgtcgggggatctaaggttgtcattctggatgaacccacagctggtgtggacccttactcccgcaggggaatatgggagctgctgctgaaataccgacaaggccgcaccattattctctctacacaccacatggatgaagcggacgtcctgggggacaggattgccatcatctcccatgggaagctgtgctgtgtgggctcctccctgtttctgaagaaccagctgggaacaggctactacctgaccttggtcaagaaagatgtggaatcctccctcagttcctgcagaaacagtagtagcactgtgtcatacctgaaaaaggaggacagtgtttctcagagcagttctgatgctggcctgggcagcgaccatgagagtgacacgctgaccatcgatgtctctgctatctccaacctcatcaggaagcatgtgtctgaagcccggctggtggaagacatagggcatgagctgacctatgtgctgccatatgaagctgctaaggagggagcctttgtggaactctttcatgagattgatgaccggctctcagacctgggcatttctagttatggcatctcagagacgaccctggaagaaatattcctcaaggtggccgaagagagtggggtggatgctgagacctcagatggtaccttgccagcaagacgaaacaggcgggccttcggggacaagcagagctgtcttcgcccgttcactgaagatgatgctgctgatccaaatgattctgacatagacccagaatccagagagacagacttgctcagtgggatggatggcaaagggtcctaccaggtgaaaggctggaaacttacacagcaacagtttgtggcccttttgtggaagagactgctaattgccagacggagtcggaaaggattttttgctcagattgtcttgccagctgtgtttgtctgcattgcccttgtgttcagcctgatcgtgccaccctttggcaagtaccccagcctggaacttcagccctggatgtacaacgaacagtacacatttgtcagcaatgatgctcctgaggacacgggaaccctggaactcttaaacgccctcaccaaagaccctggcttcgggacccgctgtatggaaggaaacccaatcccagacacgccctgccaggcaggggaggaagagtggaccactgccccagttccccagaccatcatggacctcttccagaatgggaactggacaatgcagaacccttcacctgcatgccagtgtagcagcgacaaaatcaagaagatgctgcctgtgtgtcccccaggggcaggggggctgcctcctccacaaagaaaacaaaacactgcagatatccttcaggacctgacaggaagaaacatttcggattatctggtgaagacgtatgtgcagatcatagccaaaagcttaaagaacaagatctgggtgaatgagtttaggtatggcggcttttccctgggtgtcagtaatactcaagcacttcctccgagtcaagaagttaatgatgccaccaaacaaatgaagaaacacctaaagctggccaaggacagttctgcagatcgatttctcaacagcttgggaagatttatgacaggactggacaccagaaataatgtcaaggtgtggttcaataacaagggctggcatgcaatcagctctttcctgaatgtcatcaacaatgccattctccgggccaacctgcaaaagggagagaaccctagccattatggaattactgctttcaatcatcccctgaatctcaccaagcagcagctctcagaggtggctccgatgaccacatcagtggatgtccttgtgtccatctgtgtcatctttgcaatgtccttcgtcccagccagctttgtcgtattcctgatccaggagcgggtcagcaaagcaaaacacctgcagttcatcagtggagtgaagcctgtcatctactggctctctaattttgtctgggatatgtgcaattacgttgtccctgccacactggtcattatcatcttcatctgcttccagcagaagtcctatgtgtcctccaccaatctgcctgtgctagcccttctacttttgctgtatgggtggtcaatcacacctctcatgtacccagcctcctttgtgttcaagatccccagcacagcctatgtggtgctcaccagcgtgaacctcttcattggcattaatggcagcgtggccacctttgtgctggagctgttcaccgacaataagctgaataatatcaatgatatcctgaagtccgtgttcttgatcttcccacatttttgcctgggacgagggctcatcgacatggtgaaaaaccaggcaatggctgatgccctggaaaggtttggggagaatcgctttgtgtcaccattatcttgggacttggtgggacgaaacctcttcgccatggccgtggaaggggtggtgttcttcctcattactgttctgatccagtacagattcttcatcaggcccagacctgtaaatgcaaagctatctcctctgaatgatgaagatgaagatgtgaggcgggaaagacagagaattcttgatggtggaggccagaatgacatcttagaaatcaaggagttgacgaagatatatagaaggaagcggaagcctgctgttgacaggatttgcgtgggcattcctcctggtgagtgctttgggctcctgggagttaatggggctggaaaatcatcaactttcaagatgttaacaggagataccactgttaccagaggagatgctttccttaacagaaatagtatcttatcaaacatccatgaagtacatcagaacatgggctactgccctcagtttgatgccatcacagagctgttgactgggagagaacacgtggagttctttgcccttttgagaggagtcccagagaaagaagttggcaaggttggtgagtgggcgattcggaaactgggcctcgtgaagtatggagaaaaatatgctggtaactatagtggaggcaacaaacgcaagctctctacagccatggctttgatcggcgggcctcctgtggtgtttctggatgaacccaccacaggcatggatcccaaagcccggcggttcttgtggaattgtgccctaagtgttgtcaaggaggggagatcagtagtgcttacatctcatagtatggaagaatgtgaagctctttgcactaggatggcaatcatggtcaatggaaggttcaggtgccttggcagtgtccagcatctaaaaaataggtttggagatggttatacaatagttgtacgaatagcagggtccaacccggacctgaagcctgtccaggatttctttggacttgcatttcctggaagtgttccaaaagagaaacaccggaacatgctacaataccagcttccatcttcattatcttctctggccaggatattcagcatcctctcccagagcaaaaagcgactccacatagaagactactctgtttctcagacaacacttgaccaagtatttgtgaactttgccaaggaccaaagtgatgatgaccacttaaaagacctctcattacacaaaaaccagacagtagtggacgttgcagttctcacatcttttctacaggatgagaaagtgaaagaaagctatgtatgaagaatcctgttcatacggggtggctgaaagtaaagaggnactagactttcctttgcaccatgtgaagtgttgtggagaaaagagccagaagttgatgtgggaagaagtaaactggatactgtactgatactattcaatgcaatgcaattcaatgcaatgaaaacaaaattccattacaggggcagtgcctttgtagcctatgtcttgtatggctctcaagtgaaagacttgaatttagttttttacctatacctatgtgaaactctattatggaacccaatggacatatgggtttgaactcacactttttttttttttttgttcctgtgtattctcattggggttgcaacaataattcatcaagtaatcatggccagcgattattgatcaaaatcaaaaggtaatgcacatcctcattcactaagccatgccatgcccaggagactggtttcccggtgacacatccattgctggcaatgagtgtgccagagttattagtgccaagtttttcagaaagtttgaagcaccatggtgtgtcatgctcacttttgtgaaagctgctctgctcagagtctatcaacattgaatatcagttgacagaatggtgccatgcgtggctaacatcctgctttgattccctctgataagctgttctggtggcagtaacatgcaacaaaaatgtgggtgtctctaggcacgggaaacttggttccattgttatattgtcctatgcttcgagccatgggtctacagggtcatccttatgagactcttaaatatacttagatcctggtaagaggcaaagaatcaacagccaaactgctggggctgcaagctgctgaagccagggcatgggattaaagagattgtgcgttcaaacctagggaagcctgtgcccatttgtcctgactgtctgctaacatggtacactgcatctcaagatgtttatctgacacaagtgtattatttctggctttttgaattaatctagaaaatgaaaagatggagttgtattttgacaaaaatgtttgtactttttaatgttatttggaattttaagttctatcagtgacttctgaatccttagaatggcctctttgtagaaccctgtggtatagaggagtatggccactgccccactatttttattttcttatgtaagtttgcatatcagtcatgactagtgcctagaaagcaatgtgatggtcaggatctcatgacattatatttgagtttctttcagatcatttaggatactcttaatctcacttcatcaatcaaatattttttgagtgtatgctgtagctgaaagagtatgtacgtacgtataagactagagagatattaagtctcagtacacttcctgtgccatgttattcagctcactggtttacaaatataggttgtcttgtggttgtaggagcccactgtaacaatactgggcagcctttttttttttttttaattgcaacaatgcaaaagccaagaaagtataagggtcacaagtctaaacaatgaattcttcaacagggaaaacagctagcttgaaaacttgctgaaaaacacaacttgtgtttatggcatttagtaccttcaaataattggctttgcagatattggataccccattaaatctgacagtctcaaatttttcatctcttcaatcactagtcaagaaaaatataaaaacaacaaatacttccatatggagcatttttcagagttttctaacccagtcttatttttctagtcagtaaacatttgtaaaaatactgtttcactaatacttactgttaactgtcttgagagaaaagaaaaatatgagagaactattgtttggggaagttcaagtgatctttcaatatcattactaacttcttccactttttccaaaatttgaatattaacgctaaaggtgtaagacttcagatttcaaattaatctttctatattttttaaatttacagaatattatataacccactgctgaaaaagaaaaaaatgattgttttagaagttaaagtcaatattgattttaaatataagtaatgaaggcatatttccaataactagtgatatggcatcgttgcattttacagtatcttcaaaaatacagaatttatagaataatttctcctcatttaatatttttcaaaatcaaagttatggtttcctcattttactaaaatcgtattctaattcttcattatagtaaatctatgagcaactccttacttcggttcctctgatttcaaggccatattttaaaaaatcaaaaggcactgtgaactattttgaagaaaacacaacattttaatacagattgaaaggacctcttctgaagctagaaacaatctatagttatacatcttcattaatactgtgttaccttttaaaatagtaattttttacattttcctgtgtaaacctaattgtggtagaaatttttaccaactctatactcaatcaagcaaaatttctgtatattccctgtggaatgtacctatgtgagtttcagaaattctcaaaatacgtgttcaaaaatttctgcttttgcatctttgggacacctcagaaaacttattaacaactgtgaatatgagaaatacagaagaaaataataagccctctatacataaatgcccagcacaattcattgttaaaaaacaaccaaacctcacactactgtatttcattatctgtactgaaagcaaatgctttgtgactattaaatgttgcacatcattcattcaaaaaaaaaaaaaaaaaaa

SEQ ID NO: 4 - protein, 2201 aa, Homo sapiens [sequence omitted]

SEQ ID NO: 5 - DNA, 4154 bp, Homo sapiens
taacgaggaacttttcgccggcgccgggccgcctctgaggccagggcaggacacgaacgcgcggagcggcggcggcgactgagagccggggccgcggcggcgctccctaggaagggccgtacgaggcggcgggcccggcgggcctcccggaggaggcggctgcgccatggacgagccacccttcagcgaggcggctttggagcaggcgctgggcgagccgtgcgatctggacgcggcgctgctgaccgacatcgaagacatgcttcagcttatcaacaaccaagacagtgacttccctggcctatttgacccaccctatgctgggagtggggcagggggcacagaccctgccagccccgataccagctccccaggcagcttgtctccacctcctgccacattgagctcctctcttgaagccttcctgagcgggccgcaggcagcgccctcacccctgtcccctccccagcctgcacccactccattgaagatgtacccgtccatgcccgctttctcccctgggcctggtatcaaggaagagtcagtgccactgagcatcctgcagacccccaccccacagcccctgccaggggccctcctgccacagagcttcccagccccagccccaccgcagttcagctccacccctgtgttaggctaccccagccctccgggaggcttctctacaggaagccctcccgggaacacccagcagccgctgcctggcctgccactggcttccccgccaggggtcccgcccgtctccttgcacacccaggtccagagtgtggtcccccagcagctactgacagtcacagctgcccccacggcagcccctgtaacgaccactgtgacctcgcagatccagcaggtcccggtcctgctgcagccccacttcatcaaggcagactcgctgcttctgacagccatgaagacagacggagccactgtgaaggcggcaggtctcagtcccctggtctctggcaccactgtgcagacagggcctttgccgaccctggtgagtggcggaaccatcttggcaacagtcccactggtcgtagatgcggagaagctgcctatcaaccggctcgcagctggcagcaaggccccggcctctgcccagagccgtggagagaagcgcacagcccacaacgccattgagaagcgctaccgctcctccatcaatgacaaaatcattgagctcaaggatctggtggtgggcactgaggcaaagctgaataaatctgctgtcttgcgcaaggccatcgactacattcgctttctgcaacacagcaaccagaaactcaagcaggagaacctaagtctgcgcactgctgtccacaaaagcaaatctctgaaggatctggtgtcggcctgtggcagtggagggaacacagacgtgctcatggagggcgtgaagactgaggtggaggacacactgaccccacccccctcggatgctggctcacctttccagagcagccccttgtcccttggcagcaggggcagtggcagcggtggcagtggcagtgactcggagcctgacagcccagtctttgaggacagcaaggcaaagccagagcagcggccgtctctgcacagccggggcatgctggaccgctcccgcctggccctgtgcacgctcgtcttcctctgcctgtcctgcaaccccttggcctccttgctgggggcccgggggcttcccagcccctcagataccaccagcgtctaccatagccctgggcgcaacgtgctgggcaccgagagcagagatggccctggctgggcccagtggctgctgcccccagtggtctggctgctcaatgggctgttggtgctcgtctccttggtgcttctctttgtctacggtgagccagtcacacggccccactcaggccccgccgtgtacttctggaggcatcgcaagcaggctgacctggacctggcccggggagactttgcccaggctgcccagcagctgtggctggccctgcgggcactgggccggcccctgcccacctcccacctggacctggcttgtagcctcctctggaacctcatccgtcacctgctgcagcgtctctgggtgggccgctggctggcaggccgggcagggggcctgcagcaggactgtgctctgcgagtggatgctagcgccagcgcccgagacgcagccctggtctaccataagctgcaccagctgcacaccatggggaagcacacaggcgggcacctcactgccaccaacctggcgctgagtgccctgaacctggcagagtgtgcaggggatgccgtgtctgtggcgacgctggccgagatctatgtggcggctgcattgagagtgaagaccagtctcccacgggccttgcattttctgacacgcttcttcctgagcagtgcccgccaggcctgcctggcacagagtggctcagtgcctcctgccatgcagtggctctgccaccccgtgggccaccgtttcttcgtggatggggactggtccgtgctcagtaccccatgggagagcctgtacagcttggccgggaacccagtggaccccctggcccaggtgactcagctattccgggaacatctcttagagcgagcactgaactgtgtgacccagcccaaccccagccctgggtcagctgatggggacaaggaattctcggatgccctcgggtacctgcagctgctgaacagctgttctgatgctgcgggggctcctgcctacagcttctccatcagttccagcatggccaccaccaccggcgtagacccggtggccaagtggtgggcctctctgacagctgtggtgatccactggctgcggcgggatgaggaggcggctgagcggctgtgcccgctggtggagcacctgccccgggtgctgcaggagtctgagagacccctgcccagggcagctctgcactccttcaaggctgcccgggccctgctgggctgtgccaaggcagagtctggtccagccagcctgaccatctgtgagaaggccagtgggtacctgcaggacagcctggctaccacaccagccagcagctccattgacaaggccgtgcagctgttcctgtgtgacctgcttcttgtggtgcgcaccagcctgtggcggcagcagcagcccccggccccggccccagcagcccagggcgccagcagcaggccccaggcttccgcccttgagctgcgtggcttccaacgggacctgagcagcctgaggcggctggcacagagcttccggcccgccatgcggagggtgttcctacatgaggccacggcccggctgatggcgggggccagccccacacggacacaccagctcctcgaccgcagtctgaggcggcgggcaggccccggtggcaaaggaggcgcggtggcggagctggagccgcggcccacgcggcgggagcacgcggaggccttgctgctggcctcctgctacctgccccccggcttcctgtcggcgcccgggcagcgcgtgggcatgctggctgaggcggcgcgcacactcgagaagcttggcgatcgccggctgctgcacgactgtcagcagatgctcatgcgcctgggcggtgggaccactgtcacttccagctagaccccgtgtccccggcctcagcacccctgtctctagccactttggtcccgtgcagcttctgtcctgcgtcgaagctttgaaggccgaaggcagtgcaagagactctggcctccacagttcgacctgcggctgctgtgtgccttcgcggtggaaggcccgaggggcgcgatcttgaccctaagaccggcggccatgatggtgctgacctctggtggccgatcggggcactgcaggggccgagccattttggggggcccccctccttgctctgcaggcaccttagtggcttttttcctcctgtgtacagggaagagaggggtacatttccctgtgctgacggaagccaacttggctttcccggactgcaagcagggctctgccccagaggcctctctctccgtcgtgggagagagacgtgtacatagtgtaggtcagcgtgcttagcctcctgacctgaggctcctgtgctactttgccttttgcaaactttattttcatagattgagaagttttgtacagagaattaaaaatgaaattatttata

SEQ ID NO: 6 - protein, 1147 aa, Homo sapiens [sequence omitted]

SEQ ID NO: 7 - DNA, 1470 bp, Homo sapiens
gacggtcacccgttgccagctctagcctttaaattcccggctcggggacctccacgcaccgcggctagcgccgacaaccagctagcgtgcaaggcgccgcggctcagcgcgtaccggcgggtttcgaaaccgcagtcctccggcgaccccgaactccgctccggagcctcagccccctggaaagtgatcccggcatcggagagccaagatgccggcccacttgctgcaggacgatatctctagctcctataccaccaccaccaccattacagcgcctcctccaggggtcctgcagaatggaggagataagttggagacgatgcccctctacttggaagacgacattcgccctgatataaaagatgatatatatgaccccacctacaaggataaggaaggcccaagccccaaggttgaatatgtctggagaaacatcatccttatgtctctgctacacttgggagccctgtatgggatcactttgattcctacctgcaagttctacacctggctttggggggtattctactattttgtcagtgccctgggcataacagcaggagctcatcgtctgtggagccaccgctcttacaaagctcggctgcccctacggctctttctgatcattgccaacacaatggcattccagaatgatgtctatgaatgggctcgtgaccaccgtgcccaccacaagttttcagaaacacatgctgatcctcataattcccgacgtggctttttcttctctcacgtgggttggctgcttgtgcgcaaacacccagctgtcaaagagaaggggagtacgctagacttgtctgacctagaagctgagaaactggtgatgttccagaggaggtactacaaacctggcttgctgatgatgtgcttcatcctgcccacgcttgtgccctggtatttctggggtgaaacttttcaaaacagtgtgttcgttgccactttcttgcgatatgctgtggtgcttaatgccacctggctggtgaacagtgctgcccacctcttcggatatcgtccttatgacaagaacattagcccccgggagaatatcctggtttcacttggagctgtgggtgagggcttccacaactaccaccactcctttccctatgactactctgccagtgagtaccgctggcacatcaacttcaacacattcttcattgattggatggccgccctcggtctgacctatgaccggaagaaagtctccaaggccgccatcttggccaggattaaaagaaccggagatggaaactacaagagtggctgagtttggggtccctcaggttcctttttcaaaaaccagccaggcagaggttttaatgtctgtttattaactactgaataatgctaccaggatgctaaagatgatgatgttaacccattccagtacagtattcttttaaaattcaaaagtattgaaagccaaaaaaaaaaaaaaaaaaaaaaaaa

SEQ ID NO: 8 - protein, 359 aa, Homo sapiens
MPAHLLQDDISSSYTTTTTITAPPPGVLQNGGDKLETMPLYLEDDIRPDIKDDIYDPTYKDKEGPSPKVEYVWRNIILMSLLHLGALYGITLIPTCKFYTWLWGVFYYFVSALGITAGAHRLWSHRSYKARLPLRLFLIIANTMAFQNDVYEWARDHRAHHKFSETHADPHNSRRGFFFSHVGWLLVRKHPAVKEKGSTLDLSDLEAEKLVMFQRRYYKPGLLMMCFILPTLVPWYFWGETFQNSVFVATFLRYAVVLNATWLVNSAAHLFGYRPYDKNISPRENILVSLGAVGEGFHNYHHSFPYDYSASEYRWHINFNTFFIDWMAALGLTYDRKKVSKAAILARIKRTGDGNYKSG

SEQ ID NO: 9 - DNA, 2930 bp, Homo sapiens
gaattccggtttcttcctaaaaaatgtctgatggccgctttctcggtcggcaccgccatgaatgccagcagttactctgcagagatgacggagcccaagtcggtgtgtgtctcggtggatgaggtggtgtccagcaacatggaggccactgagacggacctgctgaatggacatctgaaaaaagtagataataacctcacggaagcccagcgcttctcctccttgcctcggagggcagctgtgaacattgaattcagggacctttcctattcggttcctgaaggaccctggtggaggaagaaaggatacaagaccctcctgaaaggaatttccgggaagttcaatagtggtgagttggtggccattatgggtccttccggggccgggaagtccacgctgatgaacatcctggctggatacagggagacgggcatgaagggggccgtcctcatcaacggcctgccccgggacctgcgctgcttccggaaggtgtcctgctacatcatgcaggatgacatgctgctgccgcatctcactgtgcaggaggccatgatggtgtcggcacatctgaagcttcaggagaaggatgaaggcagaagggaaatggtcaaggagatactgacagcgctgggcttgctgtcttgcgccaacacgcggaccgggagcctgtcaggtggtcagcgcaagcgcctggccatcgcgctggagctggtgaacaaccctccagtcatgttcttcgatgagcccaccagcggcctggacagcgcctcctgcttccaggtggtctcgctgatgaaagggctcgctcaagggggtcgctccatcatttgcaccatccaccagcccagcgccaaactcttcgagctgttcgaccagctttacgtcctgagtcaaggacaatgtgtgtaccggggaaaagtctgcaatcttgtgccatatttgagggatttgggtctgaactgcccaacctaccacaacccagcagattttgtcatggaggttgcatccggcgagtacggtgatcagaacagtcggctggtgagagcggttcgggagggcatgtgtgactcagaccacaagagagacctcgggggtgatgccgaggtgaacccttttctttggcaccgcccctctgaagaggtaaagcagacaaaacgattaaaggggttgagaaaggactcctcgtccatggaaggctgccacagcttctctgccagctgcctcacgcagttctgcatcctcttcaagaggaccttcctcagcatcatgagggactcggtcctgacacacctgcgcatcacctcgcacattgggatcggcctcctcattggcctgctgtacttggggatcgggaacgaaaccaagaaggtcttgagcaactccggcttcctcttcttctccatgctgttcctcatgttcgcggccctcatgcctactgttctgacatttcccctggagatgggagtctttcttcgggaacacctgaactactggtacagcctgaaggcctactacctggccaagaccatggcagacgtgccctttcagatcatgttcccagtggcctactgcagcatcgtgtactggatgacgtcgcagccgtccgacgccgtgcgctttgtgctgtttgccgcgctgggcaccatgacctccctggtggcacagtccctgggcctgctgatcggagccgcctccacgtccctgcaggtggccactttcgtgggcccagtgacagccatcccggtgctcctgttctcggggttcttcgtcagcttcgacaccatccccacgtacctacagtggatgtcctacatctcctatgtcaggtatgggttcgaaggggtcatcctctccatctatggcttagaccgggaagatctgcactgtgacatcgacgagacgtgccacttccagaagtcggaggccatcctgcgggagctggacgtggaaaatgccaagctgtacctggacttcatcgtactcgggattttcttcatctccctccgcctcattgcctatttggtcctcaggtacaaaatccgggcagagaggtaaaacacctgaatgccaggaaacaggaagattagacactgtggccgagggcacgtctagaatcgaggaggcaagcctgtgcccgaccgacgacacagagactcttctgatccaacccctagaaccgcgttgggtttgtgggtgtctcgtgctcagccactctgcccagctgggttggatcttctctccattcccctttctagctttaactaggaagatgtaggcagattggtggttttttttttttttttaacatacagaattttaaataccacaactggggcagaatttaaagctgcaacacagctggtgatgagaggcttcctcagtccagtcgctccttagcaccaggcaccgtgggtcctggatggggaactgcaagcagcctctcagctgatggctgcacagtcagatgtctggtggcagagagtccgagcatggagcgattccattttatgactgttgtttttcacattttcatctttctaaggtgtgtctcttttccaatgagaagtcatttttgcaagccaaaagtcgatcaatcgcattcattttaagaaattatacctttttagtacttgctgaagaatgattcagggtaaatcacatactttgtttagagaggcgaggggtttaacccgagtcacccagctggtctcatacatagacagcacttgtgaaggattgaatgcaggttccaggtggagggaagacgtggacaccatctccactgagccatgcagacatttttaaaagctatacacaaaattgtgagaagacattggccaactctttcaaagtctttctttttccacgtgcttcttattttaagcgaaatatattgtttgtttcttcctaaaaaaaaaaaaaaaaaaaaa

SEQ ID NO: 10 - protein, 674 aa, Homo sapiens
MAAFSVGTAMNASSYSAEMTEPKSVCVSVDEVVSSNMEATETDLLNGHLKKVDNNLTEAQRFSSLPRRAAVNIEFRDLSYSVPEGPWWRKKGYKTLLKGISGKFNSGELVAIMGPSGAGKSTLMNILAGYRETGMKGAVLINGLPRDLRCFRKVSCYIMQDDMLLPHLTVQEAMMVSAHLKLQEKDEGRREMVKEILTALGLLSCANTRTGSLSGGQRKRLAIALELVNNPPVMFFDEPTSGLDSASCFQVVSLMKGLAQGGRSIICTIHQPSAKLFELFDQLYVLSQGQCVYRGKVCNLVPYLRDLGLNCPTYHNPADFVMEVASGEYGDQNSRLVRAVREGMCDSDHKRDLGGDAEVNPFLWHRPSEEVKQTKRLKGLRKDSSSMEGCHSFSASCLTQFCILFKRTFLSIMRDSVLTHLRITSHIGIGLLIGLLYLGIGNETKKVLSNSGFLFFSMLFLMFAALMPTVLTFPLEMGVFLREHLNYWYSLKAYYLAKTMADVPFQIMFPVAYCSIVYWMTSQPSDAVRFVLFAALGTMTSLVAQSLGLLIGAASTSLQVATFVGPVTAIPVLLFSGFFVSFDTIPTYLQWMSYISYVRYGFEGVILSIYGLDREDLHCDIDETCHFQKSEAILRELDVENAKLYLDFIVLGIFFISLRLIAYLVLRYKIRAER

SEQ ID NO: 11 - DNA, 1528 bp, Homo sapiens
cagtgccttggtaatgaccagggctccagaaagagatgtccttgtggctgggggcccctgtgcctgacattcctcctgactctgcggtggagctgtggaagccaggcgcacaggatgcaagcagccaggcccagggaggcagcagctgcatcctcagagaggaagccaggatgccccactctgctgggggtactgcaggggtggggctggaggctgcagagcccacagccctgctcaccagggcagagcccccttcagaacccacagagatccgtccacaaaagcggaaaaaggggccagcccccaaaatgctggggaacgagctatgcagcgtgtgtggggacaaggcctcgggcttccactacaatgttctgagctgcgagggctgcaagggattcttccgccgcagcgtcatcaagggagcgcactacatctgccacagtggcggccactgccccatggacacctacatgcgtcgcaagtgccaggagtgtcggcttcgcaaatgccgtcaggctggcatgcgggaggagtgtgtcctgtcagaagaacagatccgcctgaagaaactgaagcggcaagaggaggaacaggctcatgccacatccttgccccccaggcgttcctcacccccccaaatcctgccccagctcagcccggaacaactgggcatgatcgagaagctcgtcgctgcccagcaacagtgtaaccggcgctccttttctgaccggcttcgagtcacgccttggcccatggcaccagatccccatagccgggaggcccgtcagcagcgctttgcccacttcactgagctggccatcgtctctgtgcaggagatagttgactttgctaaacagctacccggcttcctgcagctcagccgggaggaccagattgccctgctgaagacctctgcgatcgaggtgatgcttctggagacatctcggaggtacaaccctgggagtgagagtatcaccttcctcaaggatttcagttataaccgggaagactttgccaaagcagggctgcaagtggaattcatcaaccccatcttcgagttctccagggccatgaatgagctgcaactcaatgatgccgagtttgccttgctcattgctatcagcatcttctctgcagaccggcccaacgtgcaggaccagctccaggtggagaggctgcagcacacatatgtggaagccctgcatgcctacgtctccatccaccatccccatgaccgactgatgttcccacggatgctaatgaaactggtgagcctccggaccctgagcagcgtccactcagagcaagtgtttgcactgcgtctgcaggacaaaaagctcccaccgctgctctctgagatctgggatgtgcacgaatgactgttctgtccccatattttctgttttcttggccggatggctgaggcctggtggctgcctcctagaagtggaacagactgagaagggcaaacattcctgggagctgggcaaggagatcctcccgtggcattaaaagagagtcaaagggt

SEQ ID NO: 12 - protein, 447 aa, Homo sapiens
MSLWLGAPVPDIPPDSAVELWKPGAQDASSQAQGGSSCILREEARMPHSAGGTAGVGLEAAEPTALLTRAEPPSEPTEIRPQKRKKGPAPKMLGNELCSVCGDKASGFHYNVLSCEGCKGFFRRSVIKGAHYICHSGGHCPMDTYMRRKCQECRLRKCRQAGMREECVLSEEQIRLKKLKRQEEEQAHATSLPPRRSSPPQILPQLSPEQLGMIEKLVAAQQQCNRRSFSDRLRVTPWPMAPDPHSREARQQRFAHFTELAIVSVQEIVDFAKQLPGFLQLSREDQIALLKTSAIEVMLLETSRRYNPGSESITFLKDFSYNREDFAKAGLQVEFINPIFEFSRAMNELQLNDAEFALLIAISIFSADRPNVQDQLQVERLQHTYVEALHAYVSIHHPHDRLMFPRMLMKLVSLRTLSSVHSEQVFALRLQDKKLPPLLSEIWDVHE

SEQ ID NO: 13 - DNA, 3188 bp, Homo sapiens
cgggcagtgacagccggcgcggatcgcgcgtccacggaggagaatcagcttagagaactatcaacacaggacaatgcaagcccatgagctgttccggtattttcgaatgccagagctggttgacttccgacagtgcgtgactcttccgaccaacacgcttatgggcttcggagctttttccagacgactcaccaccttctggcggccacgccacccaaaacccctgaagccgccatggcacctctccatgcagtcagtggaagtggcgggtagtggtggtgcacgaagatccgcactacttgacagcgacgagcccttggtgtatttctatgatgatgttacaacattatacgaaggtttccagagagggatacaggtgtcaaataatggcccttgtttaggctctcggaaaccagaccaaccctatgaatggctttcatataaacaggttgcagaattgtcggagtgcataggctcagcactgatccagaagggcttcaagactgccccagatcagttcattggcatctttgctcaaaatagacctgagtgggtgattattgaacaaggatgctttgcttattcgatggtgatcgttccactttatgatacccttggaaatgaagccatcacgtacatagtcaacaaagctgaactctctctggtttttgttgacaagccagagaaggccaaactcttattagagggtgtagaaaataagttaataccaggccttaaaatcatagttgtcatggactcgtacggcagtgaactggtggaacgaggccagaggtgtggggtggaagtcaccagcatgaaggcgatggaggacctgggaagagccaacagacggaagcccaagcctccagcacctgaagatcttgcagtaatttgtttcacaagtggaactacaggcaaccccaaaggagcaatggtcactcaccgaaacatagtgagcgattgttcagcttttgtgaaagcaacagagaatacagtcaatccttgcccagatgatactttgatatctttcttgcctctcgcccatatgtttgagagagttgtagagtgtgtaatgctgtgtcatggagctaaaatcggatttttccaaggagatatcaggctgctcatggatgacctcaaggtgcttcaacccactgtcttccccgtggttccaagactgctgaaccggatgtttgaccgaattttcggacaagcaaacaccaccgtgaagcgatggctcttggactttgcctccaagaggaaagaagcagacgttcgcagcggcatcatcagaaacaacagcctgtgggaccggctgatcttccacaaagtacagtcgagcctgggcggaagagtccggctgatggtgacaggagccgccccggtgtctgccactgtgctgacgttcctcagagcagccctgggctgtcagttttatgaaggatacggacagacagagtgcactgccgggtgctgcctaaccatgcctggagactggaccacaggccatgttggggccccgatgccgtgcaatttgataaaacttggttggcagttggaagaaatgaattacatggcgtccgagggcgagggcgaggtgtgtgtgaaagggccaaatgtatttcagggctacttgaaggacccagcgaaaacagcagaagctttggacaaagacggctggttacacacaggggacatcggaaaatggttaccaaatggcaccttgaaaattatcgaccggaaaaagcacatatttaagctggcacaaggagaatacatagcccctgaaaagattgaaaatatctacatgcgaagtgagcctgttgctcaggtgtttgtccacggagaaagcctgcaggcatttctcattgcaattgtggtaccagatgttgagacattatgttcctgggcccaaaagagaggatttgaagggtcgtttgaggaactgtgcagaaataaggatgtcaaaaaagctatcctcgaagatatggtgagacttgggaaggattctggtctgaaaccatttgaacaggtcaaaggcatcacattgcaccctgaattattttctatcgacaatggccttctgactccaacaatgaaggcgaaaaggccagagctgcggaactatttcaggtcgcagatagatgacctctattccatcatcaaggtttagtgtgaagaagaaagctcagaggaaatggcacagttccacaatctcttctcctgctgatggccttcatgttgttaattttgaatacagcaagtgtagggaaggaagcgttctgtgtttgacttgtccattcggggttcttctcataggaatgctagaggaaacagaacactgccttacagtcacctcagtgttcagaccatgtttatggtaatacacacttccaaaagtagccttaaaaattgtaaagggatactataaatgtgctaattatttgagacttcctcagtttaaaaagtgggttttaaatcttctgtctccctgtttttctaatcaaggggttaggactttgctatctctgagatgtctgctacttcgtcgaaattctgcagctgtctgctgctctaaagagtacagtgctctagagggaagtgttccctttaaaaataagaacaactgtcctggctggagatctcacaagcggaccagagatctttttaaatccctgctactgtcccttctcacaggcattcacagaacccttctgattcgaagggttacgaaactcatgttcttctccagtcccctgtggtttctgttggagcataaggtttccagtaagcgggagggcagatccaactcagaaccatgcagataaggagcctctggcaaatgggtgctgcatcagaacgcgtggattctctttcatggcagatgctcttggactcggttctccaggcctgattccccgactccatcctttttcagggttatttaaaaatctgccttagattctatagtgaagacaagcatttcaagaaagagttacctggatcagccatgctcagctgtgacgcctgataactgtctactttatcttcactgaaccactcactctgtgtaaaggccaacggatttttaatgtggttttcatatcaaaagatcatgttgggattaacttgcctttttccccaaaaaataaactctcaggcaaggcatttcttttaaagctattccg

SEQ ID NO: 14 - protein, 699 aa, Homo sapiens
MQAHELFRYFRMPELVDFRQCVTLPTNTLMGFGAFSRRLTTFWRPRHPKPLKPPWHLSMQSVEVAGSGGARRSALLDSDEPLVYFYDDVTTLYEGFQRGIQVSNNGPCLGSRKPDQPYEWLSYKQVAELSECIGSALIQKGFKTAPDQFIGIFAQNRPEWVIIEQGCFAYSMVIVPLYDTLGNEAITYIVNKAELSLVFVDKPEKAKLLLEGVENKLIPGLKIIVVMDSYGSELVERGQRCGVEVTSMKAMEDLGRANRRKPKPPAPEDLAVICFTSGTTGNPKGAMVTHRNIVSDCSAFVKATENTVNPCPDDTLISFLPLAHMFERVVECVMLCHGAKIGFFQGDIRLLMDDLKVLQPTVFPVVPRLLNRMFDRIFGQANTTVKRWLLDFASKRKEADVRSGIIRNNSLWDRLIFHKVQSSLGGRVRLMVTGAAPVSATVLTFLRAALGCQFYEGYGQTECTAGCCLTMPGDWTTGHVGAPMPCNLIKLGWQLEEMNYMASEGEGEVCVKGPNVFQGYLKDPAKTAEALDKDGWLHTGDIGKWLPNGTLKIIDRKKHIFKLAQGEYIAPEKIENIYMRSEPVAQVFVHGESLQAFLIAIVVPDVETLCSWAQKRGFEGSFEELCRNKDVKKAILEDMVRLGKDSGLKPFEQVKGITLHPELFSIDNGLLTPTMKAKRPELRNYFRSQIDDLYSIIKV

SEQ ID NO: 15 - DNA, 3634 bp, Homo sapiens
tcaacacaggacaatgcaagcccatgagctgttccggtattttcgaatgccagagctggttgacttccgacagtacgtgcgtactcttccgaccaacacgcttatgggcttcggagcttttgcagcactcaccaccttctggtacgccacgagacccaaacccctgaagccgccatgcgacctctccatgcagtcagtggaagtggcgggtagtggtggtgcacgaagatccgcactacttgacagcgacgagcccttggtgtatttctatgatgatgtcacaacattatacgaaggtttccagaggggaatacaggtgtcaaataatggcccttgtttaggctctcggaaaccagaccaaccctatgaatggctttcatataaacaggttgcagaattgtcggagtgcataggctcagcactgatccagaagggcttcaagactgccccagatcagttcattggcatctttgctcaaaatagacctgagtgggtgattattgaacaaggatgctttgcttattcgatggtgatcgttccactttatgatacccttggaaatgaagccatcacgtacatagtcaacaaagctgaactctctctggtttttgttgacaagccagagaaggccaaactcttattagagggtgtagaaaataagttaataccaggccttaaaatcatagttgtcatggatgcctacggcagtgaactggtggaacgaggccagaggtgtggggtggaagtcaccagcatgaaggcgatggaggacctgggaagagccaacagacggaagcccaagcctccagcacctgaagatcttgcagtaatttgtttcacaagtggaactacaggcaaccccaaaggagcaatggtcactcaccgaaacatagtgagcgattgttcagcttttgtgaaagcaacagagaatacagtcaatccttgcccagatgatactttgatatctttcttgcctctcgcccatatgtttgagagagttgtagagtgtgtaatgctgtgtcatggagctaaaatcggatttttccaaggagatatcaggctgctcatggatgacctcaaggtgcttcaacccactgtcttccccgtggttccaagactgctgaaccggatgtttgaccgaattttcggacaagcaaacaccacgctgaagcgatggctcttggactttgcctccaagaggaaagaagcagagcttcgcagcggcatcatcagaaacaacagcctgtgggaccggctgatcttccacaaagtacagtcgagcctgggcggaagagtccggctgatggtgacaggagccgccccggtgtctgccactgtgctgacgttcctcagagcagccctgggctgtcagttttatgaaggatacggacagacagagtgcactgccgggtgctgcctaaccatgcctggagactggaccgcaggccatgttggggccccgatgccgtgcaatttgataaaacttgttgatgtggaagaaatgaattacatggctgccgagggcgagggcgaggtgtgtgtgaaagggccaaatgtatttcagggctacttgaaggacccagcgaaaacagcagaagctttggacaaagacggctggttacacacaggggacattggaaaatggttaccaaatggcaccttgaaaattatcgaccggaaaaagcacatatttaagctggcacaaggagaatacatagcccctgaaaagattgaaaatatctacatgcgaagtgagcctgttgctcaggtgtttgtccacggagaaagcctgcaggcatttctcattgcaattgtggtaccagatgttgagacattatgttcctgggcccaaaagagaggatttgaagggtcgtttgaggaactgtgcagaaataaggatgtcaaaaaagctatcctcgaagatatggtgagacttgggaaggattctggtctgaaaccatttgaacaggtcaaaggcatcacattgcaccctgaattattttctatcgacaatggccttctgactccaacaatgaaggcgaaaaggccagagctgcggaactatttcaggtcgcagatagatgacctctattccactatcaaggtttagtgtgaagaagaaagctcagaggaaatggcacagttccacaatctcttctcctgctgatggccttcatgttgttaattttgaatacagcaagtgtagggaaggaagcgttcgtgtttgacttgtccattcggggttcttctcataggaatgctagaggaaacagaacaccgccttacagtcacctcatgttgcagaccatgtttatggtaatacacactttccaaaatgagccttaaaaattgtaaaggggatactataaatgtgctaagttatttgagacttcctcagtttaaaaagtgggttttaaatcttctgtctccctgcttttctaatcaaggggttaggactttgctatctctgagatgtctgctacttgctgcaaattctgcagctgtctgctgctctaaagagtacagtgcactagagggaagtgttccctttaaaaataagaacaactgtcctggctggagaatctcacaagcggaccagagatctttttaaatccctgctactgtcccttctcacaggcattcacagaacccttctgattcgtaagggttacgaaactcatgttcttctccagtcccctgtggtttctgttggagcataaggtttccagtaagcgggagggcagatccaactcagaaccatgcagataaggagcctctggcaaatgggtgctcatcagaacgcgtggattctctttcatggcagaatgctcttggactcggttctccaggcctgattccccgactccatcctttttcaggggttatttaaaaatctgccttagattctatagtgaagacaagcatttcaagaaagagttacctggatcagccatgctcagctgtgacgcctgaataactgtctactttatcttcactgaaccactcactctgtgtaaaggccaacagatttttaatgtggttttcatatcaaaagatcatgttgggattaacttgcctttttccccaaaaaataaactctcaggcaagcatttctttaaagctattaagggagtatatacttgagtacttattgaaatggacagtaataagcaaatgttcttataatgctacctgatttctatgaaatgtgtttgacaagccaaaattctaggatgtagaaatctggaaagttcatttcctgggattcacttctccagggattttttaaagttaatttgggaaattaacagcagttcactttattgtgagtctttgccacatttgactgaattgagctgtcatttgtacatttaaagcagctgttttggggtctgtgagagtacatgtattatatacaagcacaacagggcttgcactaaagaattgtcattgtaataacactacttggtagcctaacttcatatatgtattcttaattgcacaaaaagtcaataatttgtcaccttggggttttgaatgtttgctttaagtgttggctatttctatgttttataaaccaaaacaaaatttccaaaaacaatgaaggaaaccaaaataaatatttctgcatttc

SEQ ID NO: 16 - protein, 698 aa, Homo sapiens
MQAHELFRYFRMPELVDFRQYVRTLPTNTLMGFGAFAALTTFWYATRPKPLKPPCDLSMQSVEVAGSGGARRSALLDSDEPLVYFYDDVTTLYEGFQRGIQVSNNGPCLGSRKPDQPYEWLSYKQVAELSECIGSALIQKGFKTAPDQFIGIFAQNRPEWVIIEQGCFAYSMVIVPLYDTLGNEAITYIVNKAELSLVFVDKPEKAKLLLEGVENKLIPGLKIIVVMDAYGSELVERGQRCGVEVTSMKAMEDLGRANRRKPKPPAPEDLAVICFTSGTTGNPKGAMVTHRNIVSDCSAFVKATENTVNPCPDDTLISFLPLAHMFERVVECVMLCHGAKIGFFQGDIRLLMDDLKVLQPTVFPVVPRLLNRMFDRIFGQANTTLKRWLLDFASKRKEAELRSGIIRNNSLWDRLIFHKVQSSLGGRVRLMVTGAAPVSATVLTFLRAALGCQFYEGYGQTECTAGCCLTMPGDWTAGHVGAPMPCNLIKLVDVEEMNYMAAEGEGEVCVKGPNVFQGYLKDPAKTAEALDKDGWLHTGDIGKWLPNGTLKIIDRKKHIFKLAQGEYIAPEKIENIYMRSEPVAQVFVHGESLQAFLIAIVVPDVETLCSWAQKRGFEGSFEELCRNKDVKKAILEDMVRLGKDSGLKPFEQVKGITLHPELFSIDNGLLTPTMKAKRPELRNYFRSQIDDLYSTIKV

SEQ ID NO: 17 - DNA, 2905 bp, Homo sapiens
gaattcgttgttgggaaggactggggaaacagctgtaacatttgccaccctcagaagctgctggtcctgtgtcacaccaccttagcctcttgatcgaggaagattctcgctgaagtctgttaattctactttttgagtacttatgaataaccacgtgtcttcaaaaccatctaccatgaagctaaaacataccatcaaccctattcttttatattttatacattttctaatatcactttatactattttaacatacattccgttttattttttctccgagtcaagacaagaaaaatcaaaccgaattaaagcaaagcctgtaaattcaaaacctgattctgcatacagatctgttaatagtttggatggtttggcttcagtattataccctggatgtgatactttagataaagtttttacatatgcaaaaaacaaatttaagaacaaaagactcttgggaacacgtgaagttttaaatgaggaagatgaagtacaaccaaatggaaaaatttttaaaaaggttattcttggacagtataattggctttcctatgaagatgtctttgttcgagcctttaattttggaaatggattacagatgttgggtcagaaaccaaagaccaacatcgccatcttctgtgagaccagggccgagtggatgatagctgcacaggcgtgttttatgtataattttcagcttgttacattatatgccactctaggaggtccagccattgttcatgcattaaatgaaacagaggtgaccaacatcattactagtaaagaactcttacaaacaaagttgaaggatatagtttctttggtcccacgcctgcggcacatcatcactgttgatggaaagccaccgacctggtccgacttccccaagggcatcattgtgcataccatggctgcagtggaggccctgggagccaaggccagcatggaaaaccaacctcatagcaaaccattgccctcagatattgcagtaatcatgtacacaagtggatccacaggacttccaaagggagtcatgatctcacatagtaacattattgctggtataactgggatggcagaaaggattccagaactaggagaggaagatgtctacattggatatttgcctctggcccatgttctagaattaagtgctgagcttgtctgtctttctcacggatgccgcattggttactcttcaccacagactttagcagatcagtcttcaaaaattaaaaaaggaagcaaaggggatacatccatgttgaaaccaacactgatggcagcagttccggaaatcatggatcggatctacaaaaatgtcatgaataaagtcagtgaaatgagtagttttcaacgtaatctgtttattctggcctataattacaaaatggaacagatttcaaaaggacgtaatactccactgtgcgacagctttgttttccggaaagttcgaagcttgctagggggaaatattcgtctcctgttgtgtggtggcgctccactttctgcaaccacgcagcgattcatgaacatctgtttctgctgtcctgttggtcagggatacgggctcactgaatctgctggggctggaacaatttccgaagtgtgggactacaatactggcagagtgggagcaccattagtttgctgtgaaatcaaattaaaaaactgggaggaaggtggatactttaatactgataagccacaccccaggggtgaaattcttattgggggccaaagtgtgacaatggggtactacaaaaatgaagcaaaaacaaaagctgatttctctgaagatgaaaatggacaaaggtggctctgtactggggatattggagagtttgaacccgatggatgcttaaagattattgatcgtaaaaaggaccttgtaaaactacaggcaggggaatatgtttctcttgggaaagtagaggcagctttgaagaatcttccactagtagataacatttgtgcatatgcaaacagttatcattcttatgtcattggatttgttgtgccaaatcaaaaggaactaactgaactagctcgaaagaaaggacttaaagggacttgggaggagctgtgtaacagttgtgaaatggaaaatgaggtacttaaagtgctttccgaagctgctatttcagcaagtctggaaaagtttgaaattccagtaaaaattcgtttgagtcctgaaccgtggacccctgaaactggtctggtgacagatgccttcaagctgaaacgcaaagagcttaaaacacattaccaggcggacattgagcgaatgtatggaagaaaataattattctcttctggcatcagtttgctacagtgagctcacatcaaataggaaaatacttgaaatgcatgtctcaagctgcaaggcaaactccattcctcatattaaactattacttctcatgacgtcaccatttttaactgacaggattagtaaaacattaagacagcaaacttgtgtctgtctcttctttcattttccccgccaccaacttactttaccacctatgactgtacttgtcagtatgagaatttttctgaatcatattggggaagcagtgattttaaaacctcaagtttttaaacatgatttatatgttctgtataatgttcagtttgtaactttttaaaagtttggatgtatagagggataaataggaaatataagaattggttatttgggggcttttttacttactgtatttaaaaatacaagggtattgatatgaaattatgtaaatttcaaatgcttatgaatcaaatcattgttgaacaaaagatttgttgctgtgtaattattgtcttgtatgcatttgagagaaataaatatacccatacttatgttttaagaagttgagatcttgtgaaaaaaaaaaaaaaaaaa

SEQ ID NO: 18 - protein, 720 aa, Homo sapiens
MNNHVSSKPSTMKLKHTINPILLYFIHFLISLYTILTYIPFYFFSESRQEKSNRIKAKPVNSKPDSAYRSVNSLDGLASVLYPGCDTLDKVFTYAKNKFKNKRLLGTREVLNEEDEVQPNGKIFKKVILGQYNWLSYEDVFVRAFNFGNGLQMLGQKPKTNIAIFCETRAEWMIAAQACFMYNFQLVTLYATLGGPAIVHALNETEVTNIITSKELLQTKLKDIVSLVPRLRHIITVDGKPPTWSDFPKGIIVHTMAAVEALGAKASMENQPHSKPLPSDIAVIMYTSGSTGLPKGVMISHSNIIAGITGMAERIPELGEEDVYIGYLPLAHVLELSAELVCLSHGCRIGYSSPQTLADQSSKIKKGSKGDTSMLKPTLMAAVPEIMDRIYKNVMNKVSEMSSFQRNLFILAYNYKMEQISKGRNTPLCDSFVFRKVRSLLGGNIRLLLCGGAPLSATTQRFMNICFCCPVGQGYGLTESAGAGTISEVWDYNTGRVGAPLVCCEIKLKNWEEGGYFNTDKPHPRGEILIGGQSVTMGYYKNEAKTKADFSEDENGQRWLCTGDIGEFEPDGCLKIIDRKKDLVKLQAGEYVSLGKVEAALKNLPLVDNICAYANSYHSYVIGFVVPNQKELTELARKKGLKGTWEELCNSCEMENEVLKVLSEAAISASLEKFEIPVKIRLSPEPWTPETGLVTDAFKLKRKELKTHYQADIERMYGRK

SEQ ID NO: 19 - DNA, 5043 bp, Homo sapiens
cgggattcggctggctctgccacaccaccgcgcgcccccgctccgcccgcccctccgggcgcgtcttttccgggctcgcgctgagtcccgcctccgccggctgtccgggtgcgcgcgcgccgctgcggctttttctctggcctccgccgcgcgctcctcctcgtcccagcgctagcgggcacgcggttcctttttgcgagctttccgagtgccaggcgccggccggctgcgaagacgcggtgggccgcccctccgattgaaatcacagaagatattcgtgttcttcttaagagaaaaagaggacatttaaaaacgctatggcaaagagaataaaagctaagcccacttcagacaaacctggaagtccatatcgctctgtcacacacttcgactcactagctgtaatagacatccctggagcagatactctggataaattatttgaccatgctgtatccaagtttgggaagaaggacagccttgggaccagggaaatcctaagtgaagaaaatgaaatgcagccaaatggaaaagtttttaagaagttaattcttgggaattataaatggatgaactatcttgaagtgaatcgcagagtgaataactttggtagtggactcactgcactgggactaaaaccaaagaacaccattgccatcttctgtgagaccagggccgaatggatgattgcagcacagacctgctttaagtacaactttcctcttgtgactttatatgccacacttggcaaagaagcagtagttcatgggctaaatgaatctgaggcttcctatctgattaccagtgttgaacttctggaaagtaaacttaagactgcattgttagatatcagttgtgttaaacatatcatttatgtggacaataaggctatcaataaagcagagtaccctgaaggatttgagattcacagcatgcaatcagtagaagagttgggatctaacccagaaaacttgggcattcctccaagtagaccaacgccttcagacatggccattgttatgtatactagtggttctactggccgacctaagggagtgatgatgcatcatagcaatttgatagctggaatgacaggccagtgtgaaagaatacctggactgggaccgaaggacacatatattggctacttgcctttggctcatgtgctagaactgacagcagagatatcttgctttacctatggctgcaggattggatattcttctccgcttacactctctgaccagtccagcaaaattaaaaaaggaagcaaaggagactgtactgtactgaagcccacacttatggctgctgttccggaaatcatggatagaatttataagaatgttatgagcaaagtccaagagatgaattatattcagaaaactctgttcaagatagggtatgattacaaattggaacagatcaaaaagggatatgatgcacctctttgcaatctgttactgtttaaaaaggtcaaggccctgctgggagggaatgtccgcatgatgctgtctggaggggccccgctatctcctcagacacaccgattcatgaatgtctgcttctgctgcccaattggccagggttatggactgacagaatcatgtggtgctgggacagttactgaagtaactgactatactactggcagagttggagcacctcttatttgctgtgaaattaagctaaaagactggcaagaaggcggttatacaattaatgacaagccaaaccccagaggtgaaatcgtaattggtggacagaacatctccatgggatattttaaaaatgaagagaaaacagcagaagattattctgtggatgaaaatggacaaaggtggttttgcactggtgatattggagaattccatcccgatggatgtttacagattatagatcgtaagaaagatctagtgaagttacaagcaggagagtatgtatctcttgggaaagtagaagctgcactgaagaattgtccacttattgacaacatctgtgcttttgccaaaagtgatcagtcctatgtgatcagttttgtggttcctaaccagaaaaggttgacacttttggcacaacagaaaggggtagaaggaacttgggttgatatctgcaataatcctgctatggaagctgaaatactgaaagaaattcgagaagctgcaaatgccatgaaattggagcgatttgaaattccaatcaaggttcgattaagcccagagccatggacccctgaaactggtttggtaactgatgctttcaaactgaaaaggaaggagctgaggaaccattacctcaaagacattgaacgaatgtatgggggcaaataaaatgttgttgtcttattgacagttgtgcaggaggtagcctggtggttttcaacctctagaattttaagcctttgttgaactgttagaatgtaaggtatatcattctaaagatagagtaaaaagaaaacaaaaccaaaagttattaaaattgttgtccggtttactttaacttagttttgcatagttctagtgcagctgaaattgaaaagttatttccctttagctgtgttattatagagcagaaattctgtttttaaaaattagcctaagatatacttgtttttgtaaagaaaaatatttaatgttgaacaaaataaattggagttggagtagaatgtagtttgaggaaatttgcagcttccaatgcctcttgtcttcctatttcagaagtttaaatattaagcatgacagaaaatatgtattaacactactcaaagcaaaagtgctgcagggctttaaaattctcttccaaccatttatcttgaaggaaaaattcaatagtaatataatacacaaaatcaaataataccttagaaggtattaagattataattgttgcataggttagatatagagtcattgtaatgttgtgaataattacagtgcctaaaataagaatagaacaacatatacaacaccaaaaaatatctagtaatatatttaaagggaaattgagctgctttttttgaaactttgagatctaaaaataactgtaattatttgaatgactaagaggaaagtacattttttgaaatgctgaaaattgcctttctgtgtttattcaaactgaaaagctgagaccaagagcaaggaaggtaaaaagttaacaggcaaacattttctcttagaaaaggtgataaaatcataagtatttggaattagaacccttgcacagcactgaacctgggaaagagatttaaactctgaatttatctttgataacagggattgattttaaaatgtacatgtattaaattacatttgtaatttaaggtctgtttgctgttgctgattttattcttgatcagtagtttgcatttcagaaagcctttcattttgctttaagtttagcaaagcggggttataatgaatgacttccccaatatcttgcttgaacttacagtgattaacttggatgagttttgggaagttaaagggaagaaaacactgttatcattttttcctgtttgggaagagcttagaaactggaaatactagatttgggagaagggcagagttacttgataagggacttgatgtttgtgcagtaacttgggagtgtggtttctttttgaatctttaattaaaacctgggattatatatccctgataaatattcacacttgaaccatagttactgtaaaatgcaaaaaatcttaatactgttattctttgcactttttcttaatcattttttatatatatgcatatatatatgtgtgtgtgtgtgttgcttatgttgttttgtacagatgtgggccaccattgcaacaaaatacattctttttgctctaaaatatttatgaagaaaatacttaaatgttatgtatatggtggtaataagggaaaaatcaagtattataaacaagaatgaaggtttttgtaaagatttctgttcagcgttttgcaaggtaaaattttaggcaagttttccctgaagttatgtgtatgtgagtattctcattcttcccaacttgcctttgaagagtgaaataccattattatcaagtagactactgttcagcttttattccttccctggttgtttatcccttaggaatgagtttcttagactttcccaatatgtgattttttttcccatttagaatggtgattttaaatgtgtgagtgcatgtactatcttatctcagatatttgcacccccaatctgcccccaactcccaaaagctagaacactgccaactgatctgttataggtcctttagaaacacataattaacacttaaggttgggtgctgctaattctttgcaaaaatccaaatattgttaagggaccagggagatgccactaccccttgattttccatctaaaaatatacatgtttatgtaaacaaatctttccatatccatagtgacttttcaagtatttaagcctaaagattttgatctcacatttttatacctgtttaaattgctcacagttattacatacacatcagccatcaactaaagttgtactttaaaaatttactacaatatgtacatttctaagtcaaacacttgtgacttttgctttaattccatgaatgttcctgcctccttgatatttgtatttattctttttttctctagagtagaggtataattgtgtgatatttcagaaatacagataaatgattcaaaaagtcacagttaaggagaatcatgtttctttgatcatgaataactgattagtaagtcttgcctatattttcctgatagcatatgacaaatgtttctaaggtaacaagatgagaacagataaagattgtgtggtgttttggatttggagagaaatattttaatttttaaatgcagttacaaattataatgtattcatatttgtactttctgttaaaatgcatgattgcagaattgtttagattttgtgtttattcttgatgaaaagctttgtttgttcttgtttttaagtttgcactcaaatcttaagaaataaatccacccatgttatcaaaaaaaaaaaaaaaaaaa

SEQ ID NO: 20 - protein, 670 aa, Homo sapiens
MAKRIKAKPTSDKPGSPYRSVTHFDSLAVIDIPGADTLDKLFDHAVSKFGKKDSLGTREILSEENEMQPNGKVFKKLILGNYKWMNYLEVNRRVNNFGSGLTALGLKPKNTIAIFCETRAEWMIAAQTCFKYNFPLVTLYATLGKEAVVHGLNESEASYLITSVELLESKLKTALLDISCVKHIIYVDNKAINKAEYPEGFEIHSMQSVEELGSNPENLGIPPSRPTPSDMAIVMYTSGSTGRPKGVMMHHSNLIAGMTGQCERIPGLGPKDTYIGYLPLAHVLELTAEISCFTYGCRIGYSSPLTLSDQSSKIKKGSKGDCTVLKPTLMAAVPEIMDRIYKNVMSKVQEMNYIQKTLFKIGYDYKLEQIKKGYDAPLCNLLLFKKVKALLGGNVRMMLSGGAPLSPQTHRFMNVCFCCPIGQGYGLTESCGAGTVTEVTDYTTGRVGAPLICCEIKLKDWQEGGYTINDKPNPRGEIVIGGQNISMGYFKNEEKTAEDYSVDENGQRWFCTGDIGEFHPDGCLQIIDRKKDLVKLQAGEYVSLGKVEAALKNCPLIDNICAFAKSDQSYVISFVVPNQKRLTLLAQQKGVEGTWVDICNNPAMEAEILKEIREAANAMKLERFEIPIKVRLSPEPWTPETGLVTDAFKLKRKELRNHYLKDIERMYGGK

SEQ ID NO: 21 - DNA, 4213 bp, Homo sapiens
tccactcctggagcccgcggaccccgagcacgcgcctgacagcccctgctggcccggcgcgcggcgtcgccaggccagctatggcccccgacccggtggccgccgagaccgcggctcagggacctaccccgcgctacttcacctgggacgaggtggcccagcgctcagggtgcgaggagcggtggctagtgatcgaccgtaaggtgtacaacatcagcgagttcacccgccggcatccagggggctcccgggtcatcagccactacgccgggcaggatgccacggatccctttgtggccttccacatcaacaagggccttgtgaagaagtatatgaactctctcctgattggagaactgtctccagagcagcccagctttgagcccaccaagaataaagagctgacagatgagttccgggagctgcgggccacagtggagcggatggggctcatgaaggccaaccatgtcttcttcctgctgtacctgctgcacatcttgctgctggatggtgcagcctggctcaccctttgggtctttgggacgtcctttttgcccttcctcctctgtgcggtgctgctcagtgcagttcaggcccaggctggctggctgcagcatgactttgggcacctgtcggtcttcagcacctcaaagtggaaccatctgctacatcattttgtgattggccacctgaagggggcccccgccagttggtggaaccacatgcacttccagcaccatgccaagcccaactgcttccgcaaagacccagacatcaacatgcatcccttcttctttgccttggggaagatcctctctgtggagcttgggaaacagaagaaaaaatatatgccgtacaaccaccagcacaaatacttcttcctaattgggcccccagccttgctgcctctctacttccagtggtatattttctattttgttatccagcgaaagaagtgggtggacttggcctggatgattaccttctacgtccgcttcttcctcacttatgtgccactattggggctgaaagccttcctgggccttttcttcatagtcaggttcctggaaagcaactggtttgtgtgggtgacacagatgaaccatattcccatgcacattgatcatgaccggaacatggactgggtttccacccagctccaggccacatgcaatgtccacaagtctgccttcaatgactggttcagtggacacctcaacttccagattgagcaccatctttttcccacgatgcctcgacacaattaccacaaagtggctcccctggtgcagtccttgtgtgccaagcatggcatagagtaccagtccaagcccctgctgtcagccttcgccgacatcatccactcactaaaggagtcagggcagctctggctagatgcctatcttcaccaataacaacagccaccctgcccagtctggaagaagaggaggaagactctggagccaaggcagaggggagcttgagggacaatgccactatagtttaatactcagagggggttgggtttggggacataaagcctctgactcaaactcctcccttttatcttctagccacagttctaagacccaaagtggggggtggacacagaagtccctaggagggaaggagctgttggggcaggggtgtaaattatttcctttttctagtttggcacatgcaggtagttggtgaacagagagaaccaggagggtaacagaagaggagggacctactgaacccagagtcaggaagagatttaacactaaaattccactcatgccgggcgtggtggcacgcgcctgtaatcccagctacccaggaggctgaggcaggagaatcgcttgaaccggggaggtggaggttgcagtgagctgagatcacgccattgtactccagcctgggcgacagagcaagactccatttcaaaaaaaaaaaaaaaatccactcatataaaaggtgagctcagctcactggtccatttctcagtggcttctccatcctcatttgcaaacctcagagggataaggcagttgaacctgatgagcaagaattataacagcaaggaaacattaatgcttagaattctgagatccagcacaactcagtctgtgggagctcagctcgctgcccagggataggtatgacctatgtctgccttaggctgctgggagatgccattctccagtttcagaagcaggcagggcaaaggtcaagactgtggtattggggtcttttggctctgaaggatcctggaaccactgattttggtttattccctccagggtctaaagagaacaagaggtgctagctcttaccaaaacagatggtagagagagttgctggctatttaaaaagctctttcatcttttaattcacctcttcttttcacctctttaaccactcctcaggaacagaacacttctaggactgggggtcttttagctccataagcaagtgagcagatgggacaagttagtcttttctccctagaaacaaaggggatgcccagtggtttccctttgcttcccaacctaaaatttcaagtttaataaaatagcaattagcagaagtgaccaaattgggagataattatcagtcatgaggaaagacacagatttcggtcataaagaatgtaagggctataagtagaaactttctataacctaaatgatgttatagaattatttttgagcaggagcagaaagattaaatatgatcacttcatacttctaaatcagaaataggaagattaaaaccacagaacagtttgtgatttctattgctgtagctaggtatcttactctgtccactcttgttcaagtatctaactcttctggaaaccaaataggctttagaagagattatcctatattcctatcagtataatactaaaatgtaactttttaatcatctggtttttaaaagataaacagtttagcccatctctccagagagcaaacataggaatatgactcaggagcctcctagggcttatcatcagccctcacacccgcttccccctccaacccacagcctttgcttccaggtggcaggattactactttgcctcttcagcagcatctactctaggcatattgatcattttagacactgggagaagagaacctcaaactaggaggaaaagacagagcctccacttagttttgggaggggatggcagacagtcaaggagatgagcgtcctaaggcatgttgggatagggtcagatgcaccacccatggagaggtttgtcaacacaaagacatggaaggttagaggtttgtcaacaaaaagacatggaaggttaggtttgtcaacacaaagacatggaagattagaggtttgtcaacacaaagacacaggaagaatgggctgcagaagatttagatgttttccatttgggcacattttacttagctggagaactaggtttaaaacagcctgggtaggaaaattagaagcaagctggatgcagtggctcatgcctgtaatcccaacacttttgggaggtccaggcaggaggatcacttgggcccaggaggtcaagcctgcagcgagctgagatcacaccactgcactccagcctggggtgatagaacaagaccctgtctcaaaaaaaaaaaaaaacaacaaaaacttagaattgaggagttgtacctccattggcttcctcactccaaaataggtgctgatccttcctattcctattctttgccaccttttgggtgtggtgtcaccagcctgtttagccaagtagctttgggcataggctgcccaatctgagcaaacaccagtgaggctctattgagccaagaccaagtcctcaaagcacctgaaccactgtggccttctcagcctacagcagtgtggtctcttacatggccacaaagggacacacagtgacaaaaggctcggaatgttacaatggtaaaatgagtgatctcaaatccactgacagatataaaataggcttagagaggaaaagctgcctctggtcaagtagatcatggcagcatgaattccaactcacttttttacaactccaacttctatgtttatctttgttactttcacttttttacaacctggccagaggcattttttaaatcaggcccaatatcagtattctttttgtgtgtgccaattttgttatcacatccctatgaagttgaaaaataaagttaattttgaccaaaaaaaaaaaaaaaag

SEQ ID NO: 22 - protein, 444 aa, Homo sapiens
MAPDPVAAETAAQGPTPRYFTWDEVAQRSGCEERWLVIDRKVYNISEFTRRHPGGSRVISHYAGQDATDPFVAFHINKGLVKKYMNSLLIGELSPEQPSFEPTKNKELTDEFRELRATVERMGLMKANHVFFLLYLLHILLLDGAAWLTLWVFGTSFLPFLLCAVLLSAVQAQAGWLQHDFGHLSVFSTSKWNHLLHHFVIGHLKGAPASWWNHMHFQHHAKPNCFRKDPDINMHPFFFALGKILSVELGKQKKKYMPYNHQHKYFFLIGPPALLPLYFQWYIFYFVIQRKKWVDLAWMITFYVRFFLTYVPLLGLKAFLGLFFIVRFLESNWFVWVTQMNHIPMHIDHDRNMDWVSTQLQATCNVHKSAFNDWFSGHLNFQIEHHLFPTMPRHNYHKVAPLVQSLCAKHGIEYQSKPLLSAFADIIHSLKESGQLWLDAYLHQ

SEQ ID NO: 23 - DNA, 1275 bp, Homo sapiens
tgtactttaacccgttttaaagtacgtttgccattctcagggcaactggattgactcggagctctaggcttatcatcgccttaaaccggttccccttccacccacgctttgctttccagtggcaggatactactttgcctcttcgcagcatctactctagcatatgatcatttagacactgggaaaagaacctcaaactaggaggaaaagacagagcctccacttagttttgggaggggatggcagacagtcaaggagatgagcgtcctaaggcatgttgggatagggtcagatgcaccacccatggagaggtttgtcaacacaaagacatggaaggttagaggtttgtcaacaaaaagacatggaaggttaggtttgtcaacacaaagacatggaagattagaggtttgtcaacacaaagacacaggaagaatgggctgcagaagatttagatgttttccatttgggcacattttacttagctggagaactaggtttaaaacagcctgggtaggaaaattagaagcaagctggatgcagtggctcatgcctgtaatcccaacacttttgggaggtccaggcaggaggatcacttgggcccaggaggtcaagcctgcagcgagctgagatcacaccactgcactccagcctggggtgatagaacaagaccctgtctcaaaaaaaaaaaaaacaacaaaaacttagaattgaggagttgtacctccattggcttcctcactccaaaataggtgctgatccttcctattcctattctttgccaccttttgggtgtggtgtcaccagcctgtttagccaagtagctttgggcataggctgcccaatctgagcaaacaccagtgaggctctattgagccaagaccaagtcctcaaagcacctgaaccactgtggccttctcagcctacagcagtgtggtctcttacatggccacaaagggacacacagtgacaaaaggctcggaatgttacaatggtaaaatgagtgatctcaaatccactgacagatataaaataggcttagagaggaaaagctgcctctggtcaagtagatcatggcagcatgaattccaactcacttttttacaactccaacttctatgtttatctttgttactttcacttttttacaacctggccagaggcattttttaaatcaggcccaatatcagtattctttttgtgtgtgccaattttgttatcacatccctatgaagttgaaaaataaagttaattttgaccaaaagaaaaaaaaaaaaa

SEQ ID NO: 24 - protein, 122 aa, Homo sapiens
MSVLRHVGIGSDAPPMERFVNTKTWKVRGLSTKRHGRLGLSTQRHGRLEVCQHKDTGRMGCRRFRCFPFGHILLSWRTRFKTAWVGKLEASWMQWLMPVIPTLLGGPGRRITWAQEVKPAAS

SEQ ID NO: 25 - DNA, 3016 bp, Homo sapiens
ttagactggcagcatggggaagggagggaaccagggcgagggggccgccgagcgcgaggtgtcggtgcccaccttcagctgggaggagattcagaagcataacctgcgcaccgacaggtggctggtcattgaccgcaaggtttacaacatcaccaaatggtccatccagcacccggggggccagcgggtcatcgggcactacgctggagaagatgcaacggatgccttccgcgccttccaccctgacctggaattcgtgggcaagttcttgaaacccctgctgattggtgaactggccccggaggagcccagccaggaccacggcaagaactcaaagatcactgaggacttccgggccctgaggaagacggctgaggacatgaacctgttcaagaccaaccacgtgttcttcctcctcctcctggcccacatcatcgccctggagagcattgcatggttcactgtcttttactttggcaatggctggattcctaccctcatcacggcctttgtccttgctacctctcaggcccaagctggatggctgcaacatgattatggccacctgtctgtctacagaaaacccaagtggaaccaccttgtccacaaattcgtcattggccacttaaagggtgcctctgccaactggtggaatcatcgccacttccagcaccacgccaagcctaacatcttccacaaggatcccgatgtgaacatgctgcacgtgtttgttctgggcgaatggcagcccatcgagtacggcaagaagaagctgaaatacctgccctacaatcaccagcacgaatacttcttcctgattgggccgccgctgctcatccccatgtatttccagtaccagattatcatgaccatgatcgtccataagaactgggtggacctggcctgggccgtcagctactacatccggttcttcatcacctacatccctttctacggcatcctgggagccctccttttcctcaacttcatcaggttcctggagagccactggtttgtgtgggtcacacagatgaatcacatcgtcatggagattgaccaggaggcctaccgtgactggttcagtagccagctgacagccacctgcaacgtggagcagtccttcttcaacgactggttcagtggacaccttaacttccagattgagcaccacctcttccccaccatgccccggcacaacttacacaagatcgccccgctggtgaagtctctatgtgccaagcatggcattgaataccaggagaagccgctactgagggccctgctggacatcatcaggtccctgaagaagtctgggaagctgtggctggacgcctaccttcacaaatgaagccacagcccccgggacactgtggggaaggggtgcaggtggggtgatggccagaggaatgatgggcttttgttctgaggggtgtccgagaggctggtgtatgcactgctcacggaccccatgttggatctttctccctttctcctctcctttttctcttcacatctcccccatagcaccctgccctcatgggacctgccctccctcagccgtcagccatcagccatggccctcccagtgcctcctagccccttcttccaaggagcagagaggtggccaccgggggtggctctgtcctacctccactctctgcccctaaagatgggaggagaccagcggtccatgggtctggcctgtgagtctccccttgcagcctggtcactaggcatcacccccgctttggttcttcagatgctcttggggttcataggggcaggtcctagtcgggcagggcccctgaccctcccggcctggcttcactctccctgacggctgccattggtccaccctttcatagagaggcctgctttgttacaaagctcgggtctccctcctgcagctcggttaagtacccgaggcctctcttaagatgtccagggccccaggcccgcgggcacagccagcccaaaccttgggccctggaagagtcctccaccccatcactagagtgctctgaccctgggctttcacgggccccattccaccgcctccccaacttgagcctgtgaccttgggaccaaagggggagtccctcgtctcttgtgactcagcagaggcagtggccacgttcagggaggggccggctggcctggaggctcagcccaccctccagcttttcctcagggtgtcctgaggtccaagattctggagcaatctgacccttctccaaaggctctgttatcagctgggcagtgccagccaatccctggccatttggccccaggggacgtgggccctgcaggctgcaggagggcactggagctgggaggtctcgtcccagccctccccatctcggggctgctgtgtggacggcgctgcctcaggcactctcctgtctgaacctgcccttactgtgtttaacctgttgctccaggatgcattctgataggagggggcggcagggctgggccttgtgacaatctgcctttcaccacatggccttgcctcggtggccctgactgtcagggagggccagggaggcagagcgggagggagtctcaggaggaggcttgccctgaggggctggggagggggtacctcatgaggaccagggtggagcttgagaagaggaggaggtgggggcttggaggtgcttggtagctgaggggacgggcaagtgagaggggagggagggaagtcctgggaggatcctgagctgctgttgcagtctaacccactaatcagttcttagattcaggggaagggcaggcaccaacaactcagaatgggggctttcggggagggcgcctagtccccccagctctaagcagccaggagggacctgcatctaagcatctgggttgccatggcaatggcatgccccccagctactgtatgcccccgacccccgcagaggcagaatgaacccatagggagctgatcgtaatgtttatcatgttacttccccacccctacattttttgaaataaaataaggaattttaaaaaa

SEQ ID NO: 26 - protein, 444 aa, Homo sapiens
MGKGGNQGEGAAEREVSVPTFSWEEIQKHNLRTDRWLVIDRKVYNITKWSIQHPGGQRVIGHYAGEDATDAFRAFHPDLEFVGKFLKPLLIGELAPEEPSQDHGKNSKITEDFRALRKTAEDMNLFKTNHVFFLLLLAHIIALESIAWFTVFYFGNGWIPTLITAFVLATSQAQAGWLQHDYGHLSVYRKPKWNHLVHKFVIGHLKGASANWWNHRHFQHHAKPNIFHKDPDVNMLHVFVLGEWQPIEYGKKKLKYLPYNHQHEYFFLIGPPLLIPMYFQYQIIMTMIVHKNWVDLAWAVSYYIRFFITYIPFYGILGALLFLNFIRFLESHWFVWVTQMNHIVMEIDQEAYRDWFSSQLTATCNVEQSFFNDWFSGHLNFQIEHHLFPTMPRHNLHKIAPLVKSLCAKHGIEYQEKPLLRALLDIIRSLKKSGKLWLDAYLHK

SEQ ID NO: 27 - DNA, 417 bp, Homo sapiens
acctcccaaccaagccctccagcaaggattcaggagtgcccctcgggcctcgccatgaggctcttcctgtcgctcccggtcctggtggtggttctgtcgatcgtcttggaaggcccagccccagcccaggggaccccagacgtctccagtgccttggataagctgaaggagtttggaaacacactggaggacaaggctcgggaactcatcagccgcatcaaacagagtgaactttctgccaagatgcgggagtggttttcagagacatttcagaaagtgaaggagaaactcaagattgactcatgaggacctgaagggtgacatccaggaggggcctctgaaatttcccacaccccagcgcctgtgctgaggactcccgccatgtggccccaggtgccaccaataaaaatcctaccg

SEQ ID NO: 28 - protein, 83 aa, Homo sapiens
MRLFLSLPVLVVVLSIVLEGPAPAQGTPDVSSALDKLKEFGNTLEDKARELISRIKQSELSAKMREWFSETFQKVKEKLKIDS

SEQ ID NO: 29 - DNA, 503 bp, Homo sapiens
gtgcccgtccggagctggtgaggacagcctgccagagtctggtctctggacactatgggcacacgactcctcccagctctgtttcttgtcctcctggtattgggatttgaggtccaggggacccaacagccccagcaagatgagatgcctagcccgaccttcctcacccaggtgaaggaatctctctccagttactgggagtcagcaaagacagccgcccagaacctgtacgagaagacatacctgcccgctgtagatgagaaactcagggacttgtacagcaaaagcacagcagccatgagcacttagacaggcatttttactgaccaagttctttctgtgctgaagggagaggagtaacagccagacacagccagactggacaaggggagagtcccctactcccctgatcccccaggttcagactgagctcccccttcccagtagctcttgcatcctcctcccaactctagcctgaattcttttcaataaaaaatacaatt

SEQ ID NO: 30 - protein, 84 aa, Homo sapiens
MGTRLLPALFLVLLVLGFEVQGTQQPQQDEMPSPTFLTQVKESLSSYWESAKTAAQNLYEKTYLPAVDEKLRDLYSKSTAAMST

SEQ ID NO: 31 - DNA, 2597 bp, Homo sapiens
gagcagcgcgcgcaagcaggccaggggaaggtgggcgcaggtgaggggccgaggtgtgcgcaggactttagccggttgagaaggatcaagcaggcatttggagcacaggtgtctagaaacttttaaggggccggttcaagaaggaaaagttcccttctgctgtgaaactatttggcaagaggctggagggcccaatggctgcaaaattgcaacccaacattcccaaagccaagagtctagatggcgtcaccaatgacagaaccgcatctcaagggcagtggggccgtgcctgggaggtggactggttttcactggcgagcgtcatcttcctactgctgttcgcccccttcatcgtctactacttcatcatggcttgtgaccagtacagctgcgccctgaccggccctgtggtggacatcgtcaccggacatgctcggctctcggacatctgggccaagactccacctataacgaggaaagccgcccagctctataccttgtgggtcaccttccaggtgcttctgtacacgtctctccctgacttctgccataagtttctacccggctacgtaggaggcatccaggagggggccgtgactcctgcaggggttgtgaacaagtatcagatcaacggcctgcaagcctggctcctcacgcacctgctctggtttgcaaacgctcatctcctgtcctggttctcgcccaccatcatcttcgacaactggatcccactgctgtggtgcgccaacatccttggctatgccgtctccaccttcgccatggtcaagggctacttcttccccaccagcgccagagactgcaaattcacaggcaatttcttttacaactacatgatgggcatcgagtttaaccctcggatcgggaagtggtttgacttcaagctgttcttcaatgggcgccccgggatcgtcgcctggaccctcatcaacctgtccttcgcagcgaagcagcgggagctccacagccatgtgaccaatgccatggtcctggtcaacgtcctgcaggccatctacgtgattgacttcttctggaacgaaacctggtacctgaagaccattgacatctgccatgaccacttcgggtggtacctgggctggggcgactgtgtctggctgccttatctttacacgctgcagggtctgtacttggtgtaccaccccgtgcagctgtccaccccgcacgccgtgggcgtcctgctgctgggcctggtgggctactacatcttccgggtggccaaccaccagaaggacctgttccgccgcacggatgggcgctgcctcatctggggcaggaagcccaaggtcatcgagtgctcctacacatccgccgacgggcagaggcaccacagcaagctgctggtgtcgggcttctggggcgtggcccgccacttcaactacgtcggcgacctgatgggcagcctggcctactgcctggcctgtggcggtggccacctgctgccctacttctacatcatctacatggccatcctgctgacccaccgctgcctccgggacgagcaccgctgcgccagcaagtacggccgggactgggagcgctacaccgccgcagtgccttaccgcctgctgcctggaatcttctaagggcacgccctagggagaagccctgtggggctgtcaagagcgtgttctgccaggtccatgggggctggcatcccagctccaactcgaggagcctcagtttcctcatctgtaaactggagagagcccagcacttggcaggtgtccagtacctaatcacgctctgttccttgcttttgccttcaagggaattccgagtgtccagcactgccgtattgccagcacagacggattttctctaatcagtgtccctgggcaggaggatgacccagtcacctttactagtcctttggagacaatttacctgtattaggagcccaggccacgctacactctgcccacactggtgagcaggaggtcttcccacgccctgtcattaggctgcatttactcttgctaaataaaagtgggagtggggcgtgcgcgttatccatgtattgcctttcagctctagatccccctcccctgcctgctctgcagtcgtgggtggggcccgtgcgccgtttctccttggtagcgtgcacggtgttgaactgggacactggggagaaaggggctttcatgtcgtttccttcctgctcctgctgcacagctgccaggagtgctctgcctggagtctgcagacctcagagaggtcccagcactggctgtggctttcaggtgtaggcaggtgggctctgcttcccgattccctgtgagcgcccaccctctcgaaagaattttctgtcttgccctgtgactgtgcagactctggctcgagcaacccggggaacttcaccctcaggggcctctccacaccttctccagcgaggaggtctcagtcccagcctcgggagggcacctccttttctgtgctttcttccctgaggcattcttcctcatccctagggtgttgtgtagaactctttttaaactctatgctccgagtagagttcatctttatattaaacttcccctgttcaaaaaaaaaaaaaaaaa

SEQ ID NO: 32 - protein, 475 aa, Homo sapiens
MAAKLQPNIPKAKSLDGVTNDRTASQGQWGRAWEVDWFSLASVIFLLLFAPFIVYYFIMACDQYSCALTGPVVDIVTGHARLSDIWAKTPPITRKAAQLYTLWVTFQVLLYTSLPDFCHKFLPGYVGGIQEGAVTPAGVVNKYQINGLQAWLLTHLLWFANAHLLSWFSPTIIFDNWIPLLWCANILGYAVSTFAMVKGYFFPTSARDCKFTGNFFYNYMMGIEFNPRIGKWFDFKLFFNGRPGIVAWTLINLSFAAKQRELHSHVTNAMVLVNVLQAIYVIDFFWNETWYLKTIDICHDHFGWYLGWGDCVWLPYLYTLQGLYLVYHPVQLSTPHAVGVLLLGLVGYYIFRVANHQKDLFRRTDGRCLIWGRKPKVIECSYTSADGQRHHSKLLVSGFWGVARHFNYVGDLMGSLAYCLACGGGHLLPYFYIIYMAILLTHRCLRDEHRCASKYGRDWERYTAAVPYRLLPGIF

SEQ ID NO: 33 - DNA, 5674 bp, Homo sapiens
aaaaagaaagctaaaaggattcttcaagaaatggttgccactgtctcaccggcaatgatcagactgactgggtgggtgctgctaaaactgttcaacagcttcttttggaacattcaaattcacaaaggtcaacttgagatggttaaagctgcaactgagacgaatttgccgcttctgtttctaccagttcatagatcccatattgactatctgctgctcactttcattctcttctgccataacatcaaagcaccatacattgcttcaggcaataatctcaacatcccaatcttcagtaccttgatccataagcttgggggcttcttcatacgacgaaggctcgatgaaacaccagatggacggaaagatgttctctatagagctttgctccatgggcatatagttgaattacttcgacagcagcaattcttggagatcttcctggaaggcacacgttctaggagtggaaaaacctcttgtgctcgggcaggacttttgtcagttgtggtagatactctgtctaccaatgtcatcccagacatcttgataatacctgttggaatctcctatgatcgcattatcgaaggtcactacaatggtgaacaactgggcaaacctaagaagaatgagagcctgtggagtgtagcaagaggtgttattagaatgttacgaaaaaactatggttgtgtccgagtggattttgcacagccattttccttaaaggaatatttagaaagccaaagtcagaaaccggtgtctgctctactttccctggagcaagcgttgttaccagctatacttccttcaagacccagtgatgctgctgatgaaggtagagacacgtccattaatgagtccagaaatgcaacagatgaatccctacgaaggaggttgattgcaaatctggctgagcatattctattcactgctagcaagtcctgtgccattatgtccacacacattgtggcttgcctgctcctctacagacacaggcagggaattgatctctccacattggtcgaagacttctttgtgatgaaagaggaagtcctggctcgtgattttgacctggggttctcaggaaattcagaagatgtagtaatgcatgccatacagctgctgggaaattgtgtcacaatcacccacactagcaggaacgatgagttttttatcacccccagcacaactgtcccatcagtcttcgaactcaacttctacagcaatggggtacttcatgtctttatcatggaggccatcatagcttgcagcctttatgcagttctgaacaagaggggactggggggtcccactagcaccccacctaacctgatcagccaggagcagctggtgcggaaggcggccagcctgtgctaccttctctccaatgaaggcaccatctcactgccttgccagacattttaccaagtctgccatgaaacagtaggaaagtttatccagtatggcattcttacagtggcagagcacgatgaccaggaagatatcagtcctagtcttgctgagcagcagtgggacaagaagcttcctgaacctttgtcttggagaagtgatgaagaagatgaagacagtgactttggggaggaacagcgagattgctacctgaaggtgagccaatccaaggagcaccagcagtttatcaccttcttacagagactccttgggcctttgctggaggcctacagctctgctgccatctttgttcacaacttcagtggtcctgttccagaacctgagtatctgcaaaagttgcacaaatacctaataaccagaacagaaagaaatgttgcagtatatgctgagagtgccacatattgtcttgtgaagaatgctgtgaaaatgtttaaggatattggggttttcaaggagaccaaacaaaagagagtgtctgttttagaactgagcagcacttttctacctcaatgcaaccgacaaaaacttctagaatatattctgagttttgtggtgctgtaggtaacgtgtggcactgctggcaaatgaaggtcatgagatgagttccttgtaggtaccagcttctggctcaagagttgaaggtgccgtcgcagggtcaggcctgccctgtcccgaggtgatctcctggaagacaagtgccttctccctccatggatctgtgatcttcccagctctgcatcaacacagcagcctgcagataacacttggggggacctcagcctctattcgcaactcataatccgtagactacaagatgaaatctcaataaattatttttgagtttattaaagattgacattttaagtacaacgtttaaggactaattactgtgatggacacagaaatgtagctgtgttctggaactgaatcttacatggtatacttagtgctgctgggtaatttgttggtatattatctggttagtggttaatgcttcctttaaaaataattgagtcatccattcactctttttcagttttatctgtcaatagtagctacatttttaatgggagcaccttttatcccaaagtgctttataaattgagtggactgatatatatcacacccaggtatcactgtgctgtcctttgctgtcagatttagaaatgtttttaagagctatgtgaaaacagacaatattagtttaggtcgggaactgagatattgtaatcaaatagttaacatcaggaagttaatttggctggcaaaattctagggaaacttggccagaaaactggtgttgaaggcttttgctcatataaacaagtgccattgagtttcaaatgaccagcaaatatatttagaacccttcctgttttatgtctgtacctcgtccacccctcaggtaatacctgcctctcacaggtacagctgtttcttggaaatcctccaaccaaatagcagttttcctaacttgattagcttgagctgacagactgttagaatacagttctctggccacagctgatgagggctttctgtactgcacacagattgtgtactgcaccccagtccaggtgactggtacccactcgagttgtgccgtgcacaacctgtccagtatatgcatgtggtggccctactgactggtaatggttagaggcatttatggatttttagctttgaggaaaaaccatgacttttaacaaatttttatgggttatatgcctaaacccttatgccacatagtggtaaataattatgaaaaatggtctgttcataattggtaggtgccttttgtgagcagggagcataattattggtttattatggtaattatggtgatttttttaaatatcatgtaatgttaaaacgttttctaacagtttactgttgcttatctccaagatattatggaattaagaatttttccagatgagtgttacatagattctttgaatttagtataaaagtactgagaattaagtttgtacttccataagcttggattttaaacactgatagtatctcatgagtaatgtgtgttttgggagagggagggatgctgattgatatttcacattgtatgaaataccatgtttgaaactcatagcaataatgctatgctgttgtgatccctctcaagttctgcatttaaaatatattttttctttataggaattgatgtataccatgaagtcattgtcagttgtagtagctctgatgttgaatgagatatcatgttttagcattccattttactgactagggtagaagaacacttttcttggctacatttggaggatacccagggagtcttgggtgttccttatctggggaagcaaacatttcactagtctctttttttcatcctttaaattgtaaattaaggattactcaagctcaccattattcaagattgggactcgcttcccagtcgacactctgccctgcctgtcattgctgcaaagagctgctgctttgccaacctaagcaaagaaaatacggcttctcttgcattattttcccttttggttggtttgttttctagaagtacgttcagatgctttggggaatgcaatgtatgatttgctagctctctcaccacttaactcactgtgaggataaatatgcatgctttttgtaattaactggtgctttgaaaatcttttttaagggagaaaaatctcaaccaaagttatgctcatccagacaagctgacctttgagttaatttcagcacaactcattcttcagtgcctcatgactgaaaacaaaaaacaaaaaaacgaaagcatcttcacaatgaagcttccagatagcaccgttttgctaaaagatacattctcattgttttccaacagtgatggcttccacataaggttaaacaaactaggtgcttgtaaataatttattacagtttactctatcgcatttctgtaacatgaaatgcatgcccttcttcaggggaagactgtggtcaagttaaaaaaaaaaaacaatattaaacaacatgaaactgcagtctgtttttgaaaatgagaatgtcctaagtgattcagaagagaggagggaagttgtgcactctgaaaatgcatgaaaaacaaaggcaaaaactagtgggaaatgtgtagaactgttaactgagacggcttcgagtcttccttctggaatctgttaaatttcacaaagtcatgagggtaaatggagaaaatatttctgggattacaatgaatgtaagcccaaattgtggaattgccagtaacctggatggggaaaagcatttcccatagcactccatgtaatatgagtgctctgtgagatgttcatcagtgttttatagaaatggtgttgctgggaaaccaagtttgcacctggaaacttacaatgcactttagcgcagtaagggcttggcatccggtagtgaaaaactgtctaacccagcattgcccaaactattttgacaccaggacctttttctcctttgggatacttatgaacctctcactaatgtcctgtggagaacattttgggaaacactatgttagatagttctttaaggagacaaaacggtaatgaacagatagcactggggcagaatatgcatgcattttgtaacgtccagtgtggcgttgaatagatgtgtatttcctcccctgcagaaaataagcacagaaaattataatgtaggtgatcggagctctttcctttgatagagagaacagccccaatgatcctggctttttcactgaacgtatcagaatacatggatgaattggggtaaataaggttttaattcagatctagaagaaagtattgtacgtttgaatgcagatttttatccacagatagttgtagtgtttagacatgacaggacctatcgttgaggtttctaagacttactatgggctgtaaacctgttttttaaaactattttagaaacctgagacttgccgtctggcattttagtttaatacaaactaatgattgcatttgaaagagattcttgaccttatttctaaacgtctagagctctgaaatgtcttgatggaaggtattaaactatttgcctgttgtacaaagaaatgttaagactcgtgaaaagaattactataaggtactgtgaaataactgcgattttgtgagcaaaacatacttggaaatgctgattgatttttatgcttgttagtgtattgcaagaaacacagaaaatgtagttttgttttaataaaccaaaaattgaacat

SEQ ID NO: 34 - protein, 662 aa, Homo sapiens
KKKAKRILQEMVATVSPAMIRLTGWVLLKLFNSFFWNIQIHKGQLEMVKAATETNLPLLFLPVHRSHIDYLLLTFILFCHNIKAPYIASGNNLNIPIFSTLIHKLGGFFIRRRLDETPDGRKDVLYRALLHGHIVELLRQQQFLEIFLEGTRSRSGKTSCARAGLLSVVVDTLSTNVIPDILIIPVGISYDRIIEGHYNGEQLGKPKKNESLWSVARGVIRMLRKNYGCVRVDFAQPFSLKEYLESQSQKPVSALLSLEQALLPAILPSRPSDAADEGRDTSINESRNATDESLRRRLIANLAEHILFTASKSCAIMSTHIVACLLLYRHRQGIDLSTLVEDFFVMKEEVLARDFDLGFSGNSEDVVMHAIQLLGNCVTITHTSRNDEFFITPSTTVPSVFELNFYSNGVLHVFIMEAIIACSLYAVLNKRGLGGPTSTPPNLISQEQLVRKAASLCYLLSNEGTISLPCQTFYQVCHETVGKFIQYGILTVAEHDDQEDISPSLAEQQWDKKLPEPLSWRSDEEDEDSDFGEEQRDCYLKVSQSKEHQQFITFLQRLLGPLLEAYSSAAIFVHNFSGPVPEPEYLQKLHKYLITRTERNVAVYAESATYCLVKNAVKMFKDIGVFKETKQKRVSVLELSSTFLPQCNRQKLLEYILSFVVL

SEQ ID NO: 35 - DNA, 441 bp, Homo sapiens
atgcaggtgctaaccaagcgttaccccaagaactgcctgctgaccgtcatggaccggtatgcagccgaggtgcacaacatggagcaggtggtgatgatccccagccttctgcgggacgtgcagctgagtgggcctgggggccaggcccaggctgaggcccctgatctctacacctacttcaccatgctcaaggccatctgtgtggatgtggaccatgggctgctgccgcgggaggagtggcaggccaaggtggcaggcagcgaagagaatggaaccgcagagacagaggaagtcgaggacgagagtgcctcaggagagctggacctggaagcccagttccacctgcacttctccagcctccatcacatcctcatgcacctcaccgagaaagcccaggaggtgacaaggaaataccaggaaatgacgggacaagtttggtag

SEQ ID NO: 36 - protein, 146 aa, Homo sapiens
MQVLTKRYPKNCLLTVMDRYAAEVHNMEQVVMIPSLLRDVQLSGPGGQAQAEAPDLYTYFTMLKAICVDVDHGLLPREEWQAKVAGSEENGTAETEEVEDESASGELDLEAQFHLHFSSLHHILMHLTEKAQEVTRKYQEMTGQVW

SEQ ID NO: 37 - DNA, 5307 bp, Homo sapiens
ccacgcgcggcgccgctcggtgcagaccatgaattacgtggggcagttagccggccaggtgtttgtcaccgtgaaggagctctacaaggggctgaatcccgccacactctcagggtgcattgacatcattgtcatccgccagcccaatggaaacctccaatgctcccctttccacgtccgctttgggaagatgggggtcctgcgctcccgagagaaagtggttgacatagaaatcaatggggaatctgtggatttgcatatgaaattgggagataatggagaagcattttttgttcaagaaacagataatgatcaggaagttatccctatgcacctggccacctcccccatcctgtcagaaggagcttcgagaatggaatgccagctgaaaaggggctctgtggacaggatgagaggcctggaccccagcacgccagcccaagtgatcgctcccagcgagacgccgtcaagcagctctgtagtaaagaagagaagaaaaaggaggagaaagtcacagctggacagcctgaagagagatgacaacatgaacacatctgaggatgaggacatgttccccatcgagatgagctcggatgaggccatggagctgctggagagcagcagaactcttcctaatgatatacctccattccaagatgatattcctgaggaaaacctctccctggctgtgatttaccctcagtcagcctcataccctaattcggatagagagtggtcacccactcccagtccttccggttcccgaccttcaacacctaaaagtgattcagaattggtcagcaagtccacggaaaggacagggcagaagaacccagaaatgctttggctgtggggagagctgccgcaggctgctaagtcttcttctccacacaagatgaaagagtccagcccattgagcagtagaaaaatttgtgataaaagtcactttcaggccattcacagcgaatcttcagacacttttagtgaccaatcgccaactctggtcggtggggcacttttggaccagaacaagcctcagacagaaatgcagtttgtgaatgaagaagacctggagaccttaggagcagcagcgccactcttgcccatgatcgaggagctcaaacccccctctgccagtgtagtccagacagcaaacaagacggattctccttccaggaaaagagataaacgaagccgacatcttggtgctgacggcgtctacttggatgacctcacagacatggatcctgaagtggcggccctgtattttcccaaaaacggagatccttccggactcgcaaaacatgcaagcgacaacggagcccggtcagccaaccagtccccgcagtcggtgggcagctcgggcgtggacagtggcgtggagagcacctcggacgggctgagggacctcccttccatcgccatctccctctgcgggggcctcagcgaccaccgggagatcacgaaagatgcattcctggagcaagctgtgtcatatcaacagtttgtggacaaccccgctattatcgatgaccccaatctcgtggtaaagattgggagtaaatattataactggacaacagcagcacccctcctcctggcaatgcaggccttccagaaacctttgccaaaggccactgtggaatctatcatgagggataaaatgcccaaaaagggaggaagatggtggttttcatggaggggaagaaacaccacaatcaaggaggaaagtaagccagagcagtgcttggctggcaaggcccatagcaccggagagcaaccgccgcagctcagcttggccaccagggtaaagcatgaatcatcctccagtgatgaggagcgcgcagctgccaagccatcaaacgcaggccacctccctcttctgcctaatgtcagctacaagaagactctccggctgacttccgagcagcttaaaagcttgaagttgaagaatggccccaacgacgtggttttcagtgtcaccacgcagtaccaaggcacgtgccgctgtgagggcaccatctatctgtggaactgggatgataaagtcatcatttctgatattgatgggacaattaccagatcagatactcttggccacattttgcccacccttgggaaggattggacccatcagggcatcgctaagctgtaccataaagtgagccagaatggatataaatttctctactgttctgcccgtgccatcgggatggcggacatgacgcggggctacctgcactgggtcaacgagaggggcacggtgctgccccaggggcccctgctgctgagtcccagcagcctcttctctgccctgcacagagaagtgattgaaaagaagccagaaaagtttaaagtccagtgtttgacagacatcaaaaacctgtttttccccaacacagaacccttttatgctgcttttggaaaccgaccagctgatgtgtattcatacaagcaagtaggagtgtctttgaatagaatatttaccgtcaaccctaaaggagagctggtacaggaacatgcaaagaccaacatctcttcgtatgtgagactctgtgaagtagtcgaccacgttttcccgttgctgaaaagaagccattcttcagactttccctgttcggataccttcagtaacttcaccttttggagagagccactgccaccttttgaaaaccaggacattcattctgcctcagcgtaaaatgtcccaagcagcctcttgccagcagtgcagagcctggttgtcacccattaaaggataggtctccccggagtgcacagctccacctgggagcctggcgcgtcatcattggcctgacagcagagagaattgagaagcatttctcccctgccccaccccggggctgacatttctaagcaagataggaagggagcactttctaggctaggagttgggtgcatttgtaccgtgaaaagcattcctcagttgtggcttaatgccagttacgacgctgcctttccggcctgctccagcaagtagctactggttcacgtgcagtttggggctgtgaaacctaggcagaaggcggctgtctgagggctgtccccgcctaggacagggtcaatcgaggaatgccagatgtgcacggtttttggcaaagtagggggcacatttccattatagcaatgttagtgccaccaccttctgaacacagtggggagggctgtgaaggctcatgtgacctggatctgaggtctctgatagaaatctggacgccaccgggtccaggcctggcctcagacttggccttgtggatgggccccttacagtatttgctgactagtctcatttttaggtgataagtttttctttaattcctttggttaaagatagtctatttcattggcatatctccccccagtttttgtggctcaaggctggaatatttatgccttaatatatctatggcagacatttaagaatgcgctttatctagctcatggtaactttgcaacgccttagattaaaatgacagtaaatattactaaggcagtattttgaatgagtttgacactgccggcttccttccatccagcgaggtggtgctgacagtgtggacttgagcacacttatgccaaatgataatgatactgacttctgttgggagctctccaaagaaactggttggttttaagaaaatagtttcaagaagttcaactatattcttttagatattatgtattgttttactctgattaggttactgtgataggcatttattcatattctttctataccactgtcattaatatattaaaaagatgtatgtgttagactatcgaaagggccttattctctctttctcatagactgaccttcttttggaatttctgagtcatttattttccttagctttttccactcaaattaagggcaagcgaaaaagtaataatttggcattctttaagcctacagaatgtgattctttcacttgtttattacactggctcgtggacagaacaatttgaaaagtgaaagaattattttggtaaaagattttgctttacttttcgaagcattatttttttaaagagtgttttactccaacgattgaaacattttcctatttaaatttcatcgttagaatcacaggaggcaaaaaatggaacggttgaatgaaattttactctttctgtgaaagaaaatccacagagttgttgcctccgttgtagttggtgggccccgttagcattggatgcctttgccaaatggttcatgtggacacacaaaggcaaacagatctgccatcgatcgcagatttctgtagaaacacggatgtgcatgtgcagattcccttttgcaggtattaaaaataattaaaaatagtcctgcctgaggttgcagtgagccgagcttgcactactgcactccagcctgggtgacagagtaagactccatgtcaaaaaaaaaaaaaaaaaaaaaaagtcctgccttaactaactcctctgcgcttgttcactagtaacctaaagaggctatattcattctttatgcaatgagggtatttttgagtgaattttaactgctctgaactaagtataagctcatgggcctgcaaaggttcagacggtttctcctttgcacccaggaggaactttggctgcgagaatggggggatgtatccctcatgcagttggcatccaggcagccctctgcagcagcacaccctgcaggcggagttttcagaggatgcaattttggatcccgaattttgatgtaccttaaacttccacatcactgcaccctgaaacagagcatgctttccagaaagtcacactctcagatctgtgtcaagttcaatgtgagccctggcaaggctggcatattaacacctgccttctggcttctgaaagtgagatttgtatatgggctgcactcacgcatatacgagttggtttatctttgtgtacatgactataacccagtgatgctgaggtcatgtgctggaatgctgtatttggaccacacatttcaaagttgccctatggaaatgaatcctacttagtgacaagtcatcaaatgtttgtcacatgtgatgaagacaaatatgtatacctggcatagagaaaaatatatacctggtacattggagaaaaataattacactttcaaagagaattccctttgcaattttatgtttggatcaccactgtaagcacactttatttgcatttgatctgtatttgtatatgctgatgcaatgataaaaatcactgtaatacttcattgtgttgtactggatgcaaagctagaaaatattgcaataaatgagaccgatgaaagac

SEQ ID NO: 38 - protein, 899 aa, Homo sapiens
HARRRSVQTMNYVGQLAGQVFVTVKELYKGLNPATLSGCIDIIVIRQPNGNLQCSPFHVRFGKMGVLRSREKVVDIEINGESVDLHMKLGDNGEAFFVQETDNDQEVIPMHLATSPILSEGASRMECQLKRGSVDRMRGLDPSTPAQVIAPSETPSSSSVVKKRRKRRRKSQLDSLKRDDNMNTSEDEDMFPIEMSSDEAMELLESSRTLPNDIPPFQDDIPEENLSLAVIYPQSASYPNSDREWSPTPSPSGSRPSTPKSDSELVSKSTERTGQKNPEMLWLWGELPQAAKSSSPHKMKESSPLSSRKICDKSHFQAIHSESSDTFSDQSPTLVGGALLDQNKPQTEMQFVNEEDLETLGAAAPLLPMIEELKPPSASVVQTANKTDSPSRKRDKRSRHLGADGVYLDDLTDMDPEVAALYFPKNGDPSGLAKHASDNGARSANQSPQSVGSSGVDSGVESTSDGLRDLPSIAISLCGGLSDHREITKDAFLEQAVSYQQFVDNPAIIDDPNLVVKIGSKYYNWTTAAPLLLAMQAFQKPLPKATVESIMRDKMPKKGGRWWFSWRGRNTTIKEESKPEQCLAGKAHSTGEQPPQLSLATRVKHESSSSDEERAAAKPSNAGHLPLLPNVSYKKTLRLTSEQLKSLKLKNGPNDVVFSVTTQYQGTCRCEGTIYLWNWDDKVIISDIDGTITRSDTLGHILPTLGKDWTHQGIAKLYHKVSQNGYKFLYCSARAIGMADMTRGYLHWVNERGTVLPQGPLLLSPSSLFSALHREVIEKKPEKFKVQCLTDIKNLFFPNTEPFYAAFGNRPADVYSYKQVGVSLNRIFTVNPKGELVQEHAKTNISSYVRLCEVVDHVFPLLKRSHSSDFPCSDTFSNFTFWREPLPPFENQDIHSASA

SEQ ID NO: 39 - DNA, 1768 bp, Homo sapiens
ggcacgaggctcaggcctgacggtccgagtggagctgcgggacagcccgaacctccaggtcagccccgcggccctccatggcgctggtgcgcgcactcgtctgctgcctgctgactgcctggcactgccgctccggcctcgggctgcccgtggcgcccgcaggcggcaggaatcctcctccggcgataggacagttttggcatgtgactgacttacacttagaccctacttaccacatcacagatgaccacacaaaagtgtgtgcttcatctaaaggtgcaaatgcctccaaccctggcccttttggagatgttctgtgtgattctccatatcaacttattttgtcagcatttgattttattaaaaattctggacaagaagcatctttcatgatatggacaggggatagcccacctcatgttcctgtacctgaactctcaacagacactgttataaatgtgatcactaatatgacaaccaccatccagagtctctttccaaatctccaggttttccctgcgctgggtaatcatgactattggccacaggatcaactgcctgtagtcaccagtaaagtgtacaatgcagtagcaaacctctggaaaccatggctagatgaagaagctattagtactttaaggaaaggtggtttttattcacagaaagttacaactaatccaaaccttaggatcatcagtctaaacacaaacttgtactacggcccaaatataatgacactgaacaagactgacccagccaaccagtttgaatggctagaaagtacattgaacaactctcagcagaataaggagaaggtgtatatcatagcacatgttccagtggggtatctgccatcttcacagaacatcacagcaatgagagaatactataatgagaaattgatagatatttttcaaaaatacagtgatgtcattgcaggacaattttatggacacactcacagagacagcattatggttctttcagataaaaaaggaagtccagtaaattctttgtttgtggctcctgctgttacaccagtgaagagtgttttagaaaaacagaccaacaatcctggtatcagactgtttcagtatgatcctcgtgattataaattattggatatgttgcagtattacttgaatctgacagaggcgaatctaaagggagagtccatctggaagctggagtatatcctgacccagacctacgacattgaagatttgcagccggaaagtttatatggattagctaaacaatttacaatcctagacagtaagcagtttataaaatactacaattacttctttgtgagttatgacagcagtgtaacatgtgataagacatgtaaggcctttcagatttgtgcaattatgaatcttgataatatttcctatgcagattgcctcaaacagctttatataaagcacaattactagtatttcacagtttttgctaatagaaaatgctgattctgattctgagatcaatttgtgggaattttacataaatctttgttaattactgagtgggcaagtagacttcctgtctttgctttcttttttttttctttttgatgccttaatgtagatatctttatcattctgaattgtattatatatttaaagtgctcattaatagaatgatggatgtaaattggatgtaaatattcagtttatataattatatctaatttgtacccttgttgaaattgtcatttatacaataaagcgaattctttatctctaaaaaaaaaaaaaaaaaaaaa

SEQ ID NO: 40 - protein, 453 aa, Homo sapiens
MALVRALVCCLLTAWHCRSGLGLPVAPAGGRNPPPAIGQFWHVTDLHLDPTYHITDDHTKVCASSKGANASNPGPFGDVLCDSPYQLILSAFDFIKNSGQEASFMIWTGDSPPHVPVPELSTDTVINVITNMTTTIQSLFPNLQVFPALGNHDYWPQDQLPVVTSKVYNAVANLWKPWLDEEAISTLRKGGFYSQKVTTNPNLRIISLNTNLYYGPNIMTLNKTDPANQFEWLESTLNNSQQNKEKVYIIAHVPVGYLPSSQNITAMREYYNEKLIDIFQKYSDVIAGQFYGHTHRDSIMVLSDKKGSPVNSLFVAPAVTPVKSVLEKQTNNPGIRLFQYDPRDYKLLDMLQYYLNLTEANLKGESIWKLEYILTQTYDIEDLQPESLYGLAKQFTILDSKQFIKYYNYFFVSYDSSVTCDKTCKAFQICAIMNLDNISYADCLKQLYIKHNY

SEQ ID NO: 41 - DNA, 2643 bp, Homo sapiens
cgcatctcaggcgcagtctctaggggctgtgcgcatcctagggggggacatgtgcatctcaggggggctgctcgcatctggggggtgctgtgtgcatctcgggggggctgtgcatctagcggggtggctgtgtccgcatctggagggggctgtgcgcaacccggggggggtgttgcgcgcatctagcaggggcggctgtgcgcatttcgggggggggctgtgcatatctggggggaccgtgcttatctctgggggcggctgtgcgcatcttgaggggtgtgtacatctcggggggcctgtgcgcatcttggggggctgtgtgcatccgcgggggctgtgcgcatctcgggtgctgtgcgctgctcctctgagctctgctctttcttgcagcgtttgcctcagccatggagggcggggccgcggcagccacccccacagcactgccttactacgtggccttctcccagctgctgggcctgaccttggtggccatgaccggcgcgtggctcgggctgtaccgaggcggcattgcctgggagagcgacctgcagttcaacgcgcaccccctctgcatggtcataggcctgatcttcctgcagggaaatgccctgctggtttaccgtgtcttcaggaacgaagctaaacgcaccaccaaggtcctgcacgggctgctgcacatctttgcgctcgtcatcgccctggttggcttggtggcggtgttcgactaccacaggaagaagggctacgctgacctgtacagcctacacagctggtgcgggatccttgtctttgtcctgtactttgtgcagtggctggtgggcttcagcttcttcctgttccccggagcttcattctccctgcggagccgctaccgcccacagcacatcttctttggtgctaccatcttcctccttcccgtgggcaccgccctgctgggcctgaaggaggcactgctgttcaacctcgggggcaagtatagcgcatttgagcccgagggtgtcctggccaacgtgctgggcctgctgctggcctgcttcggtggggcggtgctctacatcttgacccgggccgactggaagcggccttcccaggcggaagagcaggccctctccatggacttcaagacgctgaggcagggagatagccccggctcccagtgatgcgcccggccggccctgggggttcgcggggtgtcttcttgcctgcccctgctgaggcgtcttcaggactgcaggctccggagagtggctctggcagcaggcgggcgggtgggtgcagggggatccgtttgatgcgtcgtttctggggcaggtctccgcctcctctgcttctcgtttctccgctgctatagaccagttcattgtgtgtggctcccgtgtctctgttgcccccttcagtgcagaaggctttgggtagacttcgggtgttcggtcctggtcgcagagcacagatctttaaagaagcgttagagaggtaggttctaccctcttggtagtagatgcctggggcaaggcccaggggaaactgggggggcctcagggacaggcctggaaaggccacgatggcctgctgaattcaaacaaggagtccctccagcctgaataacacgtggcacaaatgggcccggcctttggcagaggagcaagtgatatgatgtgtaaagtatgttggtggtgaaagcaaggttccccaggagaggggagggactggcccctgggaagctctgagatgaggctgtggcccagctgtagtcctgaccttactcttctttaaaaccctttagccctaggatggctttggtgggagaggggatagaagcccatgacttcagacagactttctcttggcagatgcaggcaggcctcctcccaggctgctccagacatgggggttggggatggggggtaccttgcagccccttcctgctggggctccctccttgtagcacccccttgcggctcagctctggtttcctctcccaggctcacccaggctctgctcaggctgggaggcagagggcacaaaccttataattttttaaatgaaaaaccgctgctgctggctgtggctagagccccctggggctgctggagctgctgcctctgttctggaggacgagccttctccttatctgctgcccatctttccaggaagtcaggatggagtcagacaactaacgatcatcccccgtggtgtctgcacatcactccagccccataaagagtgtcatgttagctgagtcaccatttggcttcggcctggaaatagtgtgttagaacactgatcgtgtgcgaggccaggagatcaagaccatcctgactaacaaacacagtgaaaccccgtctctacggaattccctgtattagtctatatggttctccaagaaactgaatgaatccattggagaagcggtggataactagccagacaaaatttgagaatacataaacaacgcattccgcaggaaacatacagaggatgccttttctgtgattgggtgggattttttccctttttatgtggatatagtagttacttgtgacaagaataattttggaataatttctattaatatcaactctgaagctaattgtacataatctcgagattgtgtttgttcataataaaagtgaagtgaatctg

SEQ ID NO: 42 - protein, 247 aa, Homo sapiens
AAAATPTALPYYVAFSQLLGLTLVAMTGAWLGLYRGGIAWESDLQFNAHPLCMVIGLIFLQGNALLVYRVFRNEAKRTTKVLHGLLHIFALVIALVGLVAVFDYHRKKGYADLYSLHSWCGILVFVLYFVQWLVGFSFFLFPGASFSLRSRYRPQHIFFGATIFLLPVGTALLGLKEALLFNLGGKYSAFEPEGVLANVLGLLLACFGGAVLYILTRADWKRPSQAEEQALSMDFKTLRQGDSPGSQ

SEQ ID NO: 43 - DNA, 2587 bp, Homo sapiens
aggcgctgcggccgtcccgggccgtgactcctcctttcccccgccccgcctccgttcggagagccggcgggcgggcgcctctcggccaggtacgcggccggctgggataggggtcgcgggcgggctttggtcgcgcagcagccggtcctccccggaggtaggcgggcgcgggccctgttgggtctttgggacgcgggtcccgctggggccggggatgctcctctctgtgagcgcggtcccgtccccccctgtccccgggcgggcgcacgggggtctaaccttggggggcggcgctcccgcctgtcccgggtgccggggttcgcgtcccgcggggccttcctcgctctttgtctcttctgagtgaacttgatgacccccttcttccaggaagcgcctcttggacgcgtgtgaccgatgcccagattgcacgaccacttctggagctgctcctgtgcgcacagcgcgaggcgccgaggccccccgcgagccagcaccgcggggctgccgcccaaggttggggagatgatcaacgtttccgtgtccgggccctccctgctggcggcccacggtgccccggacgctgaccccgcgcccaggggccgcagtgctgcgatgagcggccccgagcccggcagcccctaccccaacacctggcatcatcgcctgttgcagaggagcctcgtgctcttctcggttggggtggtcctaaccctggtgctcaacctgctgcagatccagaggaatgtcactctcttccccgaggaggtgatcgccaccatcttttcctccgcctggtgggtccctccctgctgcgggacagcagctgctgttgttggcctactgtacccctgtatcgacagtcacctcggagaaccccacaaatttaagagagaatgggccagtgtcatgcgctgcatagcaggttttggtggcattaaccacgccagtgctaaattggattttgccaataatgtccagctgtccttgactttagcagccctatctttgggcctttggtggacatttgatcgttccagaagtggccttgggctggggatcaccatagcttttctagctacgctgatcacgcagtttctcgtgtataatggtgtctatcagtatacgtccccagatttcctctatattcgttcttggctcccttgtatatttttctcaggaggcgtcacggtggggaacataggacgacagttagctatgggtgttcctgaaaagccccatagtgattgagtcttcaaaaccaccgattctgagagcaaggaagattttggaagaaaatctgactgtggattatgacaaagattatcttttttcttaagtaatctatttagatcgggctgactgtacaaatgactcctggaaaaaactcgtcacctatctagaaaagtcaagaatagggaggtggagaatgatgacttaccctgaagtcttcccttgctacccacactggcgcctgtctgtgccctggagcattctgcccagcctacgtgggttcagtcaggtgccaccttcccaagtattcgatttcattcatgtgattaaaacaagttgccatatttcaaagccttgagctaagactcaattaccaacccgcagttttgtgtcagtgcccaaaggagggaggttgatggtgcttaacaaacatgaagtatggtgtaataggaataatatttatccaaaagatttttaaaaatagggctgtgtttaaagaaggaatcaaaacaagaaaagcagcagtgattatagagaggtcacactctaagtggggtcgcggcgtggccacgcttcacggtcacgctcgtccgtcctgcagtggcgtgtttacatggtcacacgtgtgtgtatcaccagtgggtcaactgcttgtcattcctcccgcggcagtgttgtgtagacaatcttactgagcaaaaggcaatgaaaagtcttgggctcccacactgcgatatattggaattcccacctcagtttatgaagtttatttcgaaatccatagtcatctaagaatgaatacctgtctgccatgtatttcaatcttagtgagccaaaattgtttgtttgttactacagaatagagatgactgttttttggcacagccctatggaatttgcaatctgtgattgccttgtaaaaaggagagtgcatatggcactgcattaaacgtgtggtgtttctagtcaatgatattggtgagcacaatgtattcatttaatggcatagaccataccagacctaatttgcaagtattgggtcttcaaacttcaagtgcaatgtattatgaaaaccaatctgagccttgtatctcttaaatatttattccttctaacgtgtgagatgtcccgagagaagggttctccattcatttcagtgctgcctggaggaaactcggcaatgatttcttcagttgtgaagttcctttcgggttacaacctccactggaaccctcaaccttcgaaatactccagttttgggggttggggccatttacttataaatttaccgccgggtttttggaatctacatgtcttgggggcgggctcaaattcttcgaaagtggttggattaaaa

SEQ ID NO: 44 - protein, 277 aa, Homo sapiens
MPRLHDHFWSCSCAHSARRRGPPRASTAGLPPKVGEMINVSVSGPSLLAAHGAPDADPAPRGRSAAMSGPEPGSPYPNTWHHRLLQRSLVLFSVGVVLTLVLNLLQIQRNVTLFPEEVIATIFSSAWWVPPCCGTAAAVVGLLYPCIDSHLGEPHKFKREWASVMRCIAGFGGINHASAKLDFANNVQLSLTLAALSLGLWWTFDRSRSGLGLGITIAFLATLITQFLVYNGVYQYTSPDFLYIRSWLPCIFFSGGVTVGNIGRQLAMGVPEKPHSD

SEQ ID NO: 45 - DNA, 2027 bp, Homo sapiens
  misc_feature (1)..(2027): n = a,c,g, or t
gggataacgggaggaaggccggccggggccncanaggagtcccaggctcgcgtaggaggcgcgcagaccttgcaccttgcaccttcgcagcgccctgcaccccgccaccatgtgcgagctgtacagtaagcgggacactctggggctgaggaagaagcacatcgggccctcatgcaaagttttctttgcatcggatcccatcaaaatagtgagagcccagaggcagtacatgtttgatgagaacggtgaacagtacttggactgcatcaacaatgttgcccatggagtggtcaaagctgccctgaaacagatggaactgctaaatacaaattctcgattcctccacgacaacattgttgagtatgccaaacgcctttcagcaactctgccggagaaactctctgtttgttattttacaaattcaggatccgaagccaacgacttagccttacgcctggctcggcagttcagaggccaccaggatgtgatcactcttgaccatgcttaccatggtcacctatcatccttaattgagattagcccatataagtttcagaaaggaaaagatgtcaaaaaagaatttgtacatgtggcaccaactccagatacttacagaggaaaatatagagaagaccatgcagactcagccagtgcttatgcagatgaagtgaagaaaatcattgaagatgctcataacagtggaaggaagattgctgcctttattgctgaatccatgcagagttgtggcggacaaataattcctccagcaggctacttccagaaagtggcagaatatgtacacggtgcagggggtgtgtttatagctgatgaagttcaagtgggctttggcagagttgggaaacatttctggagcttccagatgtatggtgaagactttgttccagacatcgtcacaatgggaaaaccgatgggcaacggccacccggtggcatgtgtggtaacaaccaaagaaattgcagaagccttcagcagctctgggatggaatattttaatacgtatggaggaaatccagtatcttgtgctgttggtttggctgtcctggatataattgaaaatgaagaccttcaaggaaatgccaagagagtagggaattatctcactgagttactgaaaaaacagaaggctaaacacactttgataggagatattaggggcattggcctttttattggaattgatttagtgaaggaccatctgaaaaggacccctgccacagctgaagctcagcacatcatctacaagatgaaagaaaaacgagtgcttctcagtgccgatggacctcatagaaatgtacttaaaataaaaccacctatgtgcttcactgaagaagatgcaaagttcatggtggaccaacttgataggattctaacagttttagaagaagctatgggaaccaaaaccgaaagtgtgacctctgagaatactccatgcaaaacaaagatgctgaaagaagcccacatagaactgcttagggacagcaccactgactccaaagaaaatcccagcagaaagagaaatggaatgtgcacggatacacattcactgctcagtaagaggctcaagacatgactgatttgcattttaaagcaagatgcgatgtccagagttacagagaatgagtagatgtgtctcatcggttaatagctctattatacctctaaaggtggaattgtcagtttagattcataaatgaaaaggtaaatgagtaatcagaataaaccaagtgataatcaaaccatgtcaagattattagttcagactctagcctgttaattttcttagttgatttctgaagctacctgatttattctattaaattgtaagcttgcaaactcaaaataaattggcagatttacctctcatgttttaatgtgtcaaattagagagcaaagtataacaggtgccttcacttttgagacttagtgccttaaaatatgtattctataatgatttcatatataaaagtatatttattgactgtaataaaataaaatatgatgtaaac

SEQ ID NO: 46 - protein, 493 aa, Homo sapiens
MCELYSKRDTLGLRKKHIGPSCKVFFASDPIKIVRAQRQYMFDENGEQYLDCINNVAHGVVKAALKQMELLNTNSRFLHDNIVEYAKRLSATLPEKLSVCYFTNSGSEANDLALRLARQFRGHQDVITLDHAYHGHLSSLIEISPYKFQKGKDVKKEFVHVAPTPDTYRGKYREDHADSASAYADEVKKIIEDAHNSGRKIAAFIAESMQSCGGQIIPPAGYFQKVAEYVHGAGGVFIADEVQVGFGRVGKHFWSFQMYGEDFVPDIVTMGKPMGNGHPVACVVTTKEIAEAFSSSGMEYFNTYGGNPVSCAVGLAVLDIIENEDLQGNAKRVGNYLTELLKKQKAKHTLIGDIRGIGLFIGIDLVKDHLKRTPATAEAQHIIYKMKEKRVLLSADGPHRNVLKIKPPMCFTEEDAKFMVDQLDRILTVLEEAMGTKTESVTSENTPCKTKMLKEAHIELLRDSTTDSKENPSRKRNGMCTDTHSLLSKRLKT

SEQ ID NO: 47 - DNA, 1100 bp, Homo sapiens
cagctgctgcccacaccgcgtcgacgccttcactgccatccccgctgtccttgccgcccccgccatgggcctagagctgtttcttgacctggtgtcccagcccagccgcgccgtctacatcttcgccaagaagaatggcatccccttagagctgcgcaccgtggatttggtcaaagggcagcacaagagcaaggagttcttgcagatcaacagcctggggaaactgccgacgctcaaggatggtgatttcatcttgaccgaaagctcggccatcctgatttacctgagctgtaagtaccagacgccggaccactggtatccatctgacctgcaggctcgtgcccgtgttcatgagtacctgggctggcatgccgactgcatccgtggcacctttggtatacccctgtgggtccaggtgttggggccactcattggggtccaggtgcccgaggagaaggtggaacgcaacaggactgccatggaccaggccctgcaatggctggaggacaagttcctgggggacaggcccttcctcgctggccagcaggtgacactggctgatctcatggccctggaggagctgatgcagccggtggctctcggctacgaactgtttgagggacggccacgactggcagcatggcgtggacgagtggaggctttcctgggtgctgagctatgccaggaggcccacagcatcatcttgagcatcctggaacaggcggccaagaaaaccctcccaacaccctcaccagaggcctatcaggctatgctgcttcgaatcgccaggatcccctgaagggtctgggatgggggccaggagattagcaacaaggattcattctgttacttacttgcccctttttatctttccctcttgccccagtcccttctctccagcttcatgtgaagctctgcacagacaagacactcagtgtccttggcagtgctgctactcctcaggtgcagcatacataaccagtaagagactaaatctgcaatatataaagagctcctacaaatcagtaacatgaagaacactcaaaaattggcaaatgtcatcagtgttttaaacagaataaagattccaaacactttga

SEQ ID NO: 48 - protein, 244 aa, Homo sapiens
MGLELFLDLVSQPSRAVYIFAKKNGIPLELRTVDLVKGQHKSKEFLQINSLGKLPTLKDGDFILTESSAILIYLSCKYQTPDHWYPSDLQARARVHEYLGWHADCIRGTFGIPLWVQVLGPLIGVQVPEEKVERNRTAMDQALQWLEDKFLGDRPFLAGQQVTLADLMALEELMQPVALGYELFEGRPRLAAWRGRVEAFLGAELCQEAHSIILSILEQAAKKTLPTPSPEAYQAMLLRIARIP

SEQ ID NO: 49 - DNA, 3293 bp, Homo sapiens
cgctccctcccctcctagctggcttcggcggggacggcggcggcggcggcggcggcggcggcgggcggggagggcgtgcgccggccgagaggtgtcggcggcgaggcaaaggaagtttcaagtggaaggtcgtccgtcggccggcgcgtcctcctgctctcctccgcagcatcatggcggagccgagcggctcgcccgtgcacgtccagcttccccagcaggcggccccggtgacagcggcggcggcggcggccccggcggccgcgacagcagcgccggccccggcagctcccgcggccccggccccggccccggccccggccccggcggcacaggctgtcggctggcccatctgcagggacgcgtacgagctgcaggaggttatcggcagtggagctactgctgtggttcaggcagccctatgcaaacccaggcaagaacgtgtagcaataaaacggatcaacttggaaaaatgccagaccagtatggatgaactattaaaagaaattcaagccatgagtcagtgcagccatcccaacgtagtgacctattacacctcttttgtggtcaaagatgaactttggctggtcatgaaattactaagtggaggttcaatgttggatatcataaaatacattgtcaaccgaggagaacacaagaatggagttctggaagaggcaataatagcaacaattcttaaagaggttttggaaggcttagactatctacacagaaacggtcagattcacagggatttgaaagctggtaatattcttctgggtgaggatggttcagtacaaatagcagattttggggtaagtgcgttcctagcaacagggggtgatgttacccgaaataaagtaagaaaaacattcgttggcaccccatgttggatggctcctgaagtcatggaacaggtgagaggctatgacttcaaggctgacatgtggagttttggaataactgccattgaattagcaacaggagcagcgccttatcacaaatatcctcccatgaaagtgttaatgttgactttgcaaaatgatccacccactttggaaacaggggtagaggataaagaaatgatgaaaaagtacggcaagtcctttagaaaattactttcactgtgtcttcagaaagatccttccaaaaggcccacagcagcagaacttttaaaatgcaaattcttccagaaagccaagaacagagagtacctgattgagaagctgcttacaagaacaccagacatagcccaaagagccaaaaaggtaagaagagttcctgggtcaagtggtcaccttcataaaaccgaagacggggactgggagtggagtgacgacgagatggatgagaagagcgaagaagggaaagcagctttttctcaggaaaagtcacgaagagtaaaagaagaaaatccagagattgcagtgagtgccagcaccatccccgaacaaatacagtccctctctgtgcacgactctcagggcccacccaatgctaatgaagactacagagaagcttcttcttgtgccgtgaacctcgttttgagattaagaaactccagaaaggaacttaatgacatacgatttgagtttactccaggaagagatacagcagatggtgtatctcaggagctcttctctgctggcttggtggatggtcacgatgtagttatagtggctgctaatttacagaagattgtagatgatcccaaagctttaaaaacattgacatttaagttggcttctggctgtgatgggtcggagattcctgatgaagtgaagctgattgggtttgctcagttgagtgtcagctgatgtatgtcccttgatgtcaccctgatctgtcatgccccaccgccacccctactcccttcaaccctccctctttctgcccatttcctcccaccccctcactcccatttcctagcaaaatcagaagattgtgaagaggccggcttcaacaaaatgggataaaaaaataattttttaaaacttacaacactccgagttctgctttattctctagcaatccacagtacaagaacaagcaaatgccacagctgcacgactgttgctcatttttccaaaagctatttaatattcttagcaatcaatttggatatcccttaagtgaaaagaatctgaaatacactcaggtggtcttatttattggcaacaaaaggaattttctatccagaagcctatttctcctttcattgttgttatttctgttataatactttaaattgtacatctgacaatactgcctcttttatgttgtatttagaaattaatatacttataaaattaagatttattagccaaacttgaattctagttttaaaactgactgtgaattttatttttcatatatttatgcattacacaccttagctataagaaaaaaagggttttgattatatgcttcttgcagttaatctcgttatttaaacaaaaagttttgggtctgtctttggagtatttgtaacttctaaattttgaaatgactgaattaggaatttggatgcttattcttttagtctgtttgcctaaaaaccaatttacaatctgactgtctcttgggagagggaggtgccttgcaaactttcacattaagaatgtgcctgaggctgctttactctggaatagtctcagatctaaaatttcctctatataaggtggcatatgttaagttttgcttcattggaccgtttagaatgctatgtaaaatgttgccattctgttagattgctaactatatacccatctctgatttggctctccttaagtgataggatttgttattctaaaggtgataaacttgaaaatatcagaatctgagttttacttgaaattttgcagaatacccaggtggagtgaaaattggaagggttttgtgcaatgactaaaaggtaaaacgctgttaaggttcaagaatcaatactttcaacccaagtagccccctgcttgactgtatattatggaactagtaaaccttaggattttgaaaattggagtctaatctttcaaggaggtgggctcccaggatggtaccattgctctttcctagctaaccctagatatggcagctctttaatgtacttcaaaaagcaaatatatattactaaggaaaaaaagttatttataattgccttgtcataattgttaaggtgttctagagccatttgcatacaatttaatgtaatttcattccattctattgtttacacaacgattactcgaagatgactgcaaaggtaaaaggaaaataaaagtgtattgcacaatgag

SEQ ID NO: 50 - protein, 547 aa, Homo sapiens
MAEPSGSPVHVQLPQQAAPVTAAAAAAPAAATAAPAPAAPAAPAPAPAPAPAAQAVGWPICRDAYELQEVIGSGATAVVQAALCKPRQERVAIKRINLEKCQTSMDELLKEIQAMSQCSHPNVVTYYTSFVVKDELWLVMKLLSGGSMLDIIKYIVNRGEHKNGVLEEAIIATILKEVLEGLDYLHRNGQIHRDLKAGNILLGEDGSVQIADFGVSAFLATGGDVTRNKVRKTFVGTPCWMAPEVMEQVRGYDFKADMWSFGITAIELATGAAPYHKYPPMKVLMLTLQNDPPTLETGVEDKEMMKKYGKSFRKLLSLCLQKDPSKRPTAAELLKCKFFQKAKNREYLIEKLLTRTPDIAQRAKKVRRVPGSSGHLHKTEDGDWEWSDDEMDEKSEEGKAAFSQEKSRRVKEENPEIAVSASTIPEQIQSLSVHDSQGPPNANEDYREASSCAVNLVLRLRNSRKELNDIRFEFTPGRDTADGVSQELFSAGLVDGHDVVIVAANLQKIVDDPKALKTLTFKLASGCDGSEIPDEVKLIGFAQLSVS

SEQ ID NO: 51 - DNA, 1812 bp, Homo sapiens
gaagagggcagagccgtgcatggggctgctccccaggacctgagcaggaacctggagttttcagagctgcctgatcattgctacagaatgaactctagcccagctgggaccccaagtccacagccctccagggccaatgggaacatcaacctggggccttcagccaacccaaatgcccagcccacggacttcgacttcctcaaagtcatcggcaaagggaactacgggaaggtcctactggccaagcgcaagtctgatggggcgttctatgcagtgaaggtactacagaaaaagtccatcttaaagaagaaagagcagagccacatcatggcagagcgcagtgtgcttctgaagaacgtgcggcaccccttcctcgtgggcctgcgctactccttccagacacctgagaagctctacttcgtgctcgactatgtcaacgggggagagctcttcttccacctgcagcgggagcgccggttcctggagccccgggccaggttctacgctgctgaggtggccagcgccattggctacctgcactccctcaacatcatttacagggatctgaaaccagagaacattctcttggactgccagggacacgtggtgctgacggattttggcctctgcaaggaaggtgtagagcctgaagacaccacatccacattctgtggtacccctgagtacttggcacctgaagtgcttcggaaagagccttatgatcgagcagtggactggtggtgcttgggggcagtcctctacgagatgctccatggcctgccgcccttctacagccaagatgtatcccagatgtatgagaacattctgcaccagccgctacagatccccggaggccggacagtggccgcctgtgacctcctgcaaagccttctccacaaggaccagaggcagcggctgggctccaaagcagactttcttgagattaagaaccatgtattcttcagccccataaactgggatgacctgtaccacaagaggctaactccacccttcaacccaaatgtgacaggacctgctgacttgaagcattttgacccagagttcacccaggaagctgtgtccaagtccattggctgtacccctgacactgtggccagcagctctggggcctcaagtgcattcctgggattttcttatgcgccagaggatgatgacatcttggattgctagaagagaaggacctgtgaaactactgaggccagctggtattagtaaggaattaccttcagctgctaggaagagcgactcaaactaacaatggcttcaacgagaagcaggtttattttttccagcacataaaagaaaaataatgtttcggagtccaggactggcaggacaggtcatcagatactcagaggctgtatctctgccctgccaaccttgacaaatggcttccaatgttaggtttgctacaagatggttactggagctctagctgcctattttgtgtttagggaagggaaaatggaggaaaggggagaagagcaaagggcgcttttaaagagctttcccaaaagctccccccaatgacttttgcttccatctcactaaccacccacccctacctggaatggaggctgggaaatgtggcttatttgctgggtacgtgactatccctaataacaaaggggttttgaccctaagacattaggggagaatgttgggtaggcagccagccctcttttaccatagggcctcctggtgtttggattttgatctcaatgtgtaaaatgacagagatgtaacaagctcatagggtatcaatatctcttattgttctatgttgaaaaa

SEQ ID NO: 52 - protein, 367 aa, Homo sapiens
MNSSPAGTPSPQPSRANGNINLGPSANPNAQPTDFDFLKVIGKGNYGKVLLAKRKSDGAFYAVKVLQKKSILKKKEQSHIMAERSVLLKNVRHPFLVGLRYSFQTPEKLYFVLDYVNGGELFFHLQRERRFLEPRARFYAAEVASAIGYLHSLNIIYRDLKPENILLDCQGHVVLTDFGLCKEGVEPEDTTSTFCGTPEYLAPEVLRKEPYDRAVDWWCLGAVLYEMLHGLPPFYSQDVSQMYENILHQPLQIPGGRTVAACDLLQSLLHKDQRQRLGSKADFLEIKNHVFFSPINWDDLYHKRLTPPFNPNVTGPADLKHFDPEFTQEAVSKSIGCTPDTVASSSGASSAFLGFSYAPEDDDILDC

SEQ ID NO: 53 - DNA, 1603 bp, Homo sapiens
tgagatgagagctgccgacagttgggggtcaagggaggagacgccatgatccccaccttcacggctctgctctgcctcgggctgagtctgggccccaggacccacatgcaggcagggcccctccccaaacccaccctctgggctgagccaggctctgtgatcagctgggggaactctgtgaccatctggtgtcaggggaccctggaggctcgggagtaccgtctggataaagaggaaagcccagcaccctgggacagacagaacccactggagcccaagaacaaggccagattctccatcccatccatgacagaggactatgcagggagataccgctgttactatcgcagccctgtaggctggtcacagcccagtgaccccctggagctggtgatgacaggagcctacagtaaacccaccctttcagccctgccgagtcctcttgtgacctcaggaaagagcgtgaccctgctgtgtcagtcacggagcccaatggacactttccttctgatcaaggagcgggcagcccatcccctactgcatctgagatcagagcacggagctcagcagcaccaggctgaattccccatgagtcctgtgacctcagtgcacggggggacctacaggtgcttcagctcacacggcttctcccactacctgctgtcacaccccagtgaccccctggagctcatagtctcaggatccttggagggtcccaggccctcacccacaaggtccgtctcaacagctgcaggccctgaggaccagcccctcatgcctacagggtcagtcccccacagtggtctgagaaggcactgggaggtactgatcggggtcttggtggtctccatcctgcttctctccctcctcctcttcctcctcctccaacactggcgtcagggaaaacacaggacattggcccagagacaggctgatttccaacgtcctccaggggctgccgagccagagcccaaggacgggggcctacagaggaggtccagcccagctgctgacgtccagggagaaaacttctgtgctgccgtgaagaacacacagcctgaggacggggtggaaatggacactcggcagagcccacacgatgaagacccccaggcagtgacgtatgccaaggtgaaacactccagacctaggagagaaatggcctctcctccctccccactgtctggggaattcctggacacaaaggacagacaggcagaagaggacagacagatggacactgaggctgctgcatctgaagccccccaggatgtgacctacgcccagctgcacagctttaccctcagacagaaggcaactgagcctcctccatcccaggaaggggcctctccagctgagcccagtgtctatgccactctggccatccactaatccaggggggacccagaccccacaagccatggagactcaggaccccagaaggcatggaagctgcctccagtagacatcactgaaccccagccagcccagacccctgacacagaccactagaagattccgggaacgttgggagtcacctgattctgcaaagataaataatatccctgcattatcaaaataaagtagcagacctctcaattca

SEQ ID NO: 54 - protein, 448 aa, Homo sapiens
MIPTFTALLCLGLSLGPRTHMQAGPLPKPTLWAEPGSVISWGNSVTIWCQGTLEAREYRLDKEESPAPWDRQNPLEPKNKARFSIPSMTEDYAGRYRCYYRSPVGWSQPSDPLELVMTGAYSKPTLSALPSPLVTSGKSVTLLCQSRSPMDTFLLIKERAAHPLLHLRSEHGAQQHQAEFPMSPVTSVHGGTYRCFSSHGFSHYLLSHPSDPLELIVSGSLEGPRPSPTRSVSTAAGPEDQPLMPTGSVPHSGLRRHWEVLIGVLVVSILLLSLLLFLLLQHWRQGKHRTLAQRQADFQRPPGAAEPEPKDGGLQRRSSPAADVQGENFCAAVKNTQPEDGVEMDTRQSPHDEDPQAVTYAKVKHSRPRREMASPPSPLSGEFLDTKDRQAEEDRQMDTEAAASEAPQDVTYAQLHSFTLRQKATEPPPSQEGASPAEPSVYATLAIH

SEQ ID NO: 55 - DNA, 2366 bp, Homo sapiens
gaccacgcgtatcgatgtcgacccacaggattgtcacagagggcagggtggtgactgaggaccagctcctcatgcttgaggctgtggtgatgcacctcgggatccgctctgcccgctgtgtcctgggcatggagggtcagcaggtcatcctgcacctgcccctatcccagaaggggcccttctggacatgggagcctagtgcccctcgaactctgctccaggtcctacaggatccagccctgaaagacctcgtcctcacctgccccaccctgccctggcattccctgatcctgcggccccagtatgagatccaagccatcatgcacatgcgcaggaccattgtcaagatcccttctaccctggaggtcgacgtggaggacgtcaccgcctcctcccggcacgtccactttatcaaaccgctgctgctgagcgaggtcctggcctgggaaggccctttccccctgtccatggagatcctggaggttcctgagggccgccccatcttcctcagcccgtgggtgggctccttgcaaaaaggccagaggctttgcgtctatggcctagcctcaccaccctggcgggtcctggcctcaagcaagggccgcaaggtgcccaggcacttcctggtgtcagggggctaccaaggcaagctgcggcggcggccaagggagttccccacggcctatgacctcctaggtgctttccagccaggccggccactccgggtggtggccacaaaggactgtgagggcgagagggaggagaatcccgagttcacgtccctggctgtgggtgaccggctggaggtgctggggcctggccaggcccatggggcccagggcagtgacgtggatgtcttggtttgtcagcggctgagtgaccaggctggggaagatgaggaggaagagtgcaaagaggaggcagagacccagagcgggtcctgctgcccttccacttccctggcagtttcgtggaggagatgagtgacagccggcgctacagcctggcagatctgactgcccagttttcaatgccttgtgaggtcaaggtggtggccaaggacaccagccaccccaatgaccctcagaacctccttcctgggcctgcggctggaggagaagatcacagagccattcttggtggtgagcctggactctgagctgggatgtgctttgagatccctccccggaggctggacctgactgtcgtggaggccaaggggcagccagacttgccagaggggtctctccccatagccacagtggaggaagctggaacagacaccttttattattgtcttcggaagttaccagcctgtgagatccaagcccccccacccaggccccctaaaaatcagggcctcagcaagcagaggagacacagcagtgagggaggcgtcaagtcttctcaagtcttaggattgcagcaacacgttcggctgcccaaacccaaggcgaagaccttgccagagttcatcaaggatggctccagtacgtacagcaagattcctgcccacaggaagggccacaggcccgctaagccccaaaggcaggatctagatgatgatgaacatgattatgaagaaatacttgagcaatttcagaaaaccatctaagtgctggaggaaccacgcttcctaactgctgcttctcagggaatccgacaccagccaaccattttaagcctctaaaagacctcgggcaagtctcacagaaactgagctgcagacggggagtagctttgtggaaactgatttgatggacactgcaccagcttccttcaggttctagattcttgctacttagggcgggctggtttggacctaacatctcgcacgtgactccctcagcctcagagccttgggatgcagagcagctggcagggttcctctcaatcctgcaaccccagctgtcccaccggtggatgcagaggggaatccgaggccatcaaccttggtgacagcagcgcagtgccaatgctgatcacactgcatgggagattttgttaacgtctgccacccccactctcacccccaagctctaagcccccgggaggcctggactgtcttcctcatctctgtagcaccaagcctgatagatctgtatatggtaaacaggggtttaaccacatgtggttaacatggattaatgtgggaatttggcttcaagaacacaaccttaggaccttgggccccaaaagctggtggtgaaatgagaggagccaatttaagaagacccttatggagacctgaggctgcagaaactggtaggtttcatcaggtggttaaagtcgtcaaagttgtaagtgactaaccaagattatttcattttaaaaccacagaataaaaatgacacctgagcttctcc

SEQ ID NO: 56 - protein, 284 aa, Homo sapiens
MEGQQVILHLPLSQKGPFWTWEPSAPRTLLQVLQDPALKDLVLTCPTLPWHSLILRPQYEIQAIMHMRRTIVKIPSTLEVDVEDVTASSRHVHFIKPLLLSEVLAWEGPFPLSMEILEVPEGRPIFLSPWVGSLQKGQRLCVYGLASPPWRVLASSKGRKVPRHFLVSGGYQGKLRRRPREFPTAYDLLGAFQPGRPLRVVATKDCEGEREENPEFTSLAVGDRLEVLGPGQAHGAQGSDVDVLVCQRLSDQAGEDEEEECKEEAETQSGSCCPSTSLAVSWRR

SEQ ID NO: 57 - DNA, 3605 bp, Homo sapiens
ggtaaatatgtgttcattaactgagattaaccttccctgagttttctcacaccaaggtgaggaccatgtccctgtttccatcactccctctccttctcctgagtatggtggcagcgtcttactcagaaactgtgacctgtgaggatgcccaaaagacctgccctgcagtgattgcctgtagctctccaggcatcaacggcttcccaggcaaagatgggcgtgatggcaccaagggagaaaagggggaaccaggccaagggctcagaggcttacagggcccccctggaaagttggggcctccaggaaatccagggccttctgggtcaccaggaccaaagggccaaaaaggagaccctggaaaaagtccggatggtgatagtagcctggctgcctcagaaagaaaagctctgcaaacagaaatggcacgtatcaaaaagtggctgaccttctctctgggcaaacaagttgggaacaagttcttcctgaccaatggtgaaataatgacctttgaaaaagtgaaggccttgtgtgtcaagttccaggcctctgtggccacccccaggaatgctgcagagaatggagccattcagaatctcatcaaggaggaagccttcctgggcatcactgatgagaagacagaagggcagtttgtggatctgacaggaaatagactgacctacacaaactggaacgagggtgaacccaacaatgctggttctgatgaagattgtgtattgctactgaaaaatggccagtggaatgacgtcccctgctccacctcccatctggccgtctgtgagttccctatctgaagggtcatatcactcaggccctccttgtctttttactgcaacccacaggcccacagtatgcttgaaaagataaattatatcaatttcctcatatccagtattgttccttttgtgggcaatcactaaaaatgatcactaacagcaccaacaaagcaataatagtagtagtagtagttagcagcagcagtagtagtcatgctaattatataatatttttaatatatactatgaggccctatcttttgcatcctacattaattatctagtttaattaatctgtaatgctttcgatagtgttaacttgctgcagtatgaaaataagacggatttatttttccatttacaacaaacacctgtgctctgttgagccttcctttctgtttgggtagagggctcccctaatgacatcaccacagtttaataccacagctttttaccaagtttcaggtattaagaaaatctattttgtaactttctctatgaactctgttttctttctaatgagatattaaaccatgtaaagaacataaataacaaatctcaagcaaacagcttcacaaattctcacacacatacatacctatatactcactttctagattaagatatgggacatttttgactccctagaagccccgttataactcctcctagtactaactcctaggaaaatactattctgacctccatgactgcacagtaatttcgtctgtttataaacattgtatagttggaatcatattgtgtgtaatgttgtatgtcttgcttactcagaattaagtctgtgagattcattcatgtcatgtgtacaaaagtttcatccttttcattgccatgtagggttcccttatattaatattcctcagttcatccattctattgttaataggcacttaagtggcttccaatttttggccatgaggaagagaacccacgaacattcctggacttgtcttttggtggacatggtgcactaatttcactacctatccaggagtggaactggtagaggatgaggaaagcatgtattcagctttagtagatattaccagttttcctaagtgattgtatgaatttatgctcctaccggcaatgtgtggcagtcctagatgctctatgtgcttgtaaaaagtcaatgttttcagttctcttgattttcattattcctgtggatgtaaagtgatatttccccatggttttaatctgtatttccccaacatgtaataaggttgaacacttttttatatgcttattgggcacttgggtatcttcttctgtgaagtacccgttcacatttttgtattttgtttaaattagttagccaatatttttcttactgatttttaagttatttttacattctgaatatgtcctttttaatgtgtattacaaatattttgctagtttttgacttgctcctaatgttgaattttgatgaacaaaatttcctaattttgagaaagtcttatttattcatattttctttcaaaattagtgctttttgtgtcatgtttaagaaatttttgcccatcccaaaatcataagatatttttcatgattttgaaaccatgaagagatttttcatgattttgaaatcatgaagatatttttccatttttttctaatagttttattaataaacattctatctattcctggtagaatagatatccacttgagacagcactatgtaggaaagaccatttttcctccactgaactagggtggtgcatttttgtaagttaggtaactgtatgtgtgtgtgtctgtttctgggctgtctattctagtctatttgttgatgcttgtgtcaaacagtacactatcttaattattgtacatttatagttgtaactgtagtccagctttgttcttcttcaagtcaagatttccatataaatattagaaacagtttctcaatttctacaaaatcctgatgaggtttctactgggaccacattgagtctatcaatcaacttatgcagaactggcaacttactactgaatctctaatcaatgttcatcatgtatcgcttcatttaactaggatttctctaacttaattgctatgttttgagatttttagtttaaaaaccttgtatatcttgttttggtggttttagtgattttaataatatattttaaatattttttcttttctattgttgtacacagaaatacagttaagttttgtgtgtagtcttacgatgtttagtaacctcaataagtttatttcttaaatctagtaatttgtagattcctctggattttgtatatgcatagtcatgtaagctgaaaatatggcaatacttgcttcttcccaattgctttaccttttttcttaccttattgcactggttagcaaccccaatacagagaccaccagagcaggtatagactcctgaaagacaatataatgaagtgctccagtcaggcctatctaaactggattcacagctctgtcacttaattgctacatgatctagagccagttactttgtgtttcagccatgtatttgcagctgagagaaaataatcattcttatttcatgaaaattgtggggatgatgaaataagttaacacctttaaagtgtgtagtaaagtatcaggatactatattttaggtcttaatacacacagttatgccgctagatacatgctttttaatgagataatgtgatattatacataacacatatcgatttttaaaaattaaatcaaccttgctttgatggaataaactccatttagtcacaaaaaaaaaaaaaaaaaaaaaaaaaaaaaaaaaaaaa

SEQ ID NO: 58 - protein, 248 aa, Homo sapiens
MSLFPSLPLLLLSMVAASYSETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 59 - DNA, 3044 bp, Homo sapiens
cagcatcgccttttcctcccgtttctccttccactcccagctccacatcctcctcctattctcccctctcccctcttcaaacccccaccttccagttccctcacctcccctttcggctggtcccctggggcttgcagcaagagggagagagagctcctgacaggattgatggtccttccccaccctgtcctctcatccgctccctccccagcaggcacagacatccccctacaaaaggcaggagcccaggctgtgtggaaacagctgctctcagacgcctttccatttgctctctgctggctaggctgggctgtgcctctgctccctcttcctctagctgagagtgggcacctggggtaccgggcccccccacctcattccccatgaatgctgtgggaagtcctgaggggcaggagctgcacaagctggggagtggagcctgggacaaccccgcctacagtggtcccccttccccacacgggacgctgagagtctgcaccatctccagcacggggcctctccagccccaacccaagaagcctgaagatgaaccccaggagacggcatacaggacccaggtgtccagctgctgcctccatatctgtcaaggcatcagaggactttggggaacaaccctgactgagaacacagctgagaaccgggaactttatatcaagaccaccctgagggagctgttggtatatattgtgttcctggtggacatctgtctactgacctatggaatgacaagctccagtgcttattactacaccaaagtgatgtctgagctcttcttacatactccatcagacactggagtctcctttcaggccatcagcagcatggcggacttctgggattttgcccagggcccactactggacagtttgtattggaccaaatggtacaacaaccagagcctgggccatggctcccactccttcatctactatgagaacatgctgctgggggttccgaggctgcggcagctaaaggtccgcaatgactcctgtgtggtgcatgaagacttccgggaggacattctgagctgctatgatgtctactctccagacaaagaagaacaactcccctttgggcccttcaatggcacagcgtggacataccactcgcaggatgagttggggggcttctcccactggggcaggctcacaagctacagcggaggtggctactacctggaccttccaggatcccgacagggtagtgcagaggctctccgggcccttcaggaggggctgtggctggacaggggcactcgagtggtgttcatcgacttctcagtctacaatgccaatatcaatcttttctgtgtcctgaggctggtggtggagtttccagctacaggaggtgccatcccatcctggcaaatccgcacagtcaagctgatccgctatgtcagcaactgggacttctttatcgttggctgtgaggtcatcttctgcgtcttcatcttctactatgtggtggaagagatcctggagctccacattcaccggcttcgctacctcagcagcatctggaacatactggacctggtgatcatcttgctctccattgtggctgtgggcttccacatattccgaaccctcgaggtgaatcggctcatggggaagctcctgcagcagccaaacacgtatgcagactttgagttcctcgccttctggcagacacagtacaacaacatgaatgctgtcaacctcttcttcgcctggatcaagatattcaagtacatcagcttcaacaaaaccatgacccagctctcctccacgctggcccgctgtgccaaggacatcctgggcttcgccgtcatgttcttcattgttttcttcgcctatgcccaactcggctacctgcttttcgggacccaagtggaaaactttagcactttcatcaagtgcattttcactcagttccggataatcctcggggactttgactacaatgctatcgacaatgccaaccgcatcctgggccctgcctactttgtcacctatgtcttcttcgtcttcttcgtgctcctgaacatgttcctggccatcatcaatgacacatattcagaggtcaaggaggagctggctggacagaaggatgagctgcaactttctgacctcctgaaacagggctacaacaagaccctactaagactgcgtctgaggaaggagagggtttcggatgtgcagaaggtcctgcagggtggggagcaggagatccagtttgaggatttcaccaacaccttaagggaactgggacacgcagagcatgaaatcactgagctcacggccaccttcaccaagtttgacagagatgggaatcgtattctggatgagaaggaacaggaaaaaatgcgacaggacctggaggaagagagggtggccctcaacactgagattgagaaactaggccgatctattgtgagcagcccacaaggcaaatcgggtccagaggctgccagagcaggaggctgggtttcaggagaagaattctacatgctcacaaggagagttctgcagctggagactgtcctggaaggagtagtgtcccagattgatgctgtaggctcaaagctgaaaatgctggagaggaaggggtggctggctccctccccaggcgtgaaggaacaagctatttggaagcacccgcagccagccccagctgtgaccccagacccctggggagtccagggtgggcaggagagtgaggttccctataaaagagaagaggaagccttagaggagaggagactctcccgtggtgagattccaacgttgcagaggagttaagtgtgaggcactcccggagcaaagtctatgaaggatcttctgcaagaggctgcctcctggtccactgaacctggaaactgagtgggctttaaccaggagataaaaatggagcctgaagggaatcaggcaaggaaatgaactcaggattcagagatctttgaattaatatgtggtgggttctgacattattcttccataagaccatgtgggtttccatggtggctatcaataaaactccttagt

SEQ ID NO: 60 - protein, 805 aa, Homo sapiens [sequence omitted]

SEQ ID NO: 61 - DNA, 1146 bp, Homo sapiens
agggagggagtgaaggagctctctgtacccaaggaaagtgcagctgagactcagacaagattacaatgaaccaactcagcttcctgctgtttctcatagcgaccaccagaggatggagtacagatgaggctaatacttacttcaaggaatggacctgttcttcgtctccatctctgcccagaagctgcaaggaaatcaaagacgaatgtcctagtgcatttgatggcctgtattttctccgcactgagaatggtgttatctaccagaccttctgtgacatgacctctgggggtggcggctggaccctggtggccagcgtgcatgagaatgacatgcgtgggaagtgcacggtgggcgatcgctggtccagtcagcagggcagcaaagcagactacccagagggggacggcaactgggccaactacaacacctttggatctgcagaggcggccacgagcgatgactacaagaaccctggctactacgacatccaggccaaggacctgggcatctggcacgtgcccaataagtcccccatgcagcactggagaaacagctccctgctgaggtaccgcacggacactggcttcctccagacactgggacataatctgtttggcatctaccagaaatatccagtgaaatatggagaaggaaagtgttggactgacaacggcccggtgatccctgtggtctatgattttggcgacgcccagaaaacagcatcttattactcaccctatggccagcgggaattcactgcgggatttgttcagttcagggtatttaataacgagagagcagccaacgccttgtgtgctggaatgagggtcaccggatgtaacactgagcaccactgcattggtggaggaggatactttccagaggccagtccccagcagtgtggagatttttctggttttgattggagtggatatggaactcatgttggttacagcagcagccgtgagataactgaggcagctgtgcttctattctatcgttgagagttttgtgggagggaacccagacctctcctcccaaccatgagatcccaaggatggagaacaacttacccagtagctagaatgttaatggcagaagagaaaacaataaatcatattgactcaaaaaaaaaaaaaaaaa

SEQ ID NO: 62 - protein, 313 aa, Homo sapiens
MNQLSFLLFLIATTRGWSTDEANTYFKEWTCSSSPSLPRSCKEIKDECPSAFDGLYFLRTENGVIYQTFCDMTSGGGGWTLVASVHENDMRGKCTVGDRWSSQQGSKADYPEGDGNWANYNTFGSAEAATSDDYKNPGYYDIQAKDLGIWHVPNKSPMQHWRNSSLLRYRTDTGFLQTLGHNLFGIYQKYPVKYGEGKCWTDNGPVIPVVYDFGDAQKTASYYSPYGQREFTAGFVQFRVFNNERAANALCAGMRVTGCNTEHHCIGGGGYFPEASPQQCGDFSGFDWSGYGTHVGYSSSREITEAAVLLFYR

SEQ ID NO: 63 - DNA, 1635 bp, Homo sapiens
gggggtggcggggacgcgagtggcggccgcggggccccggacaagggtccgcagagctgcagccttcgagggccagccctctccgagtccggggctgggtcccaccagtgacaaggcggcagccccgcgcacaccaaagagaaagcggctgtggcggcagcggcagccccagccatgctgtgttatgtgacgaggccggacgcggtgctgatggaggtggaggtggaggcgaaagccaacggcgaggactgcctcaaccaggtgtgcaggcgactgggaatcatagaagttgactattttggactgcaatttacgggtagcaaaggtgaaagtttatggctaaacctgagaaaccggatctcccagcagatggatgggctagccccttacaggcttaaacttagagtcaagttcttcgtggagcctcatctcatcttacaggagcagactaggcatatctttttcttgcacatcaaggaggccctcttggcaggccacctcttgtgttccccagagcaggcagtggaactcagtgccctcctggcccagaccaagtttggagactacaaccagaacactgccaagtataactatgaggagctctgtgccaaggagctctcctctgccaccttgaacagcattgttgcaaaacataaggagttggaggggaccagccaggcttcagctgaataccaagttttgcagattgtgtcggcaatggaaaactatggcatagaatggcattctgtgcgggatagcgaagggcagagactgctcattggggttggacctgaaggaatctcaatttgtaaagatgactttagcccaattaataggatagcttatcctgtggtgcagatggccacccagtcaggaaagaatgtatatttgacggtcaccaaggaatctgggaacagcatcgtgctcttgtttaaaatgatcagcaccagggcggccagcgggctctaccgagcgataacagagacgcacgcattctacaggtgtgacacagtgaccagcgccgtgatgatgcagtatagccgtgacttgaagggccacttggcatctctgtttctgaatgaaaacattaaccttggcaagaaatatgtctttgatattaaaagaacatcaaaggaggtgtatgaccatgccaggagggctctgtacaatgctggcgttgtggacctcgtttcaagaagcaaccagagcccttcacactcgcctctgaagtcctcagaaagcagcatgaactgcagcagctgcgagggcctcagctgccagcagacccgggtgctgcaggagaagctacgcaagctgaaggaagccatgctgtgcatggtgtgctgcgaggaggagatcaactccaccttctgtccctgtggccacactgtgtgctgtgagagctgcgccgcccagctacagtcatgtcccgtctgcaggtcgcgtgtggagcatgtccagcacgtctatctgccaacgcacaccagtcttctcaatctgactgtaatctaatctgttgtgcttttgttggacttggcatgtttccatgaactgcactattataaactattaaaatgatagatgttggagaaagtaattattccaacacccatctgcccatgcgatgttaaaaaa

SEQ ID NO: 64 - protein, 445 aa, Homo sapiens
MLCYVTRPDAVLMEVEVEAKANGEDCLNQVCRRLGIIEVDYFGLQFTGSKGESLWLNLRNRISQQMDGLAPYRLKLRVKFFVEPHLILQEQTRHIFFLHIKEALLAGHLLCSPEQAVELSALLAQTKFGDYNQNTAKYNYEELCAKELSSATLNSIVAKHKELEGTSQASAEYQVLQIVSAMENYGIEWHSVRDSEGQRLLIGVGPEGISICKDDFSPINRIAYPVVQMATQSGKNVYLTVTKESGNSIVLLFKMISTRAASGLYRAITETHAFYRCDTVTSAVMMQYSRDLKGHLASLFLNENINLGKKYVFDIKRTSKEVYDHARRALYNAGVVDLVSRSNQSPSHSPLKSSESSMNCSSCEGLSCQQTRVLQEKLRKLKEAMLCMVCCEEEINSTFCPCGHTVCCESCAAQLQSCPVCRSRVEHVQHVYLPTHTSLLNLTVI

SEQ ID NO: 65 - DNA, 1741 bp, Homo sapiens
gaattccggacaggtagtaagataggaagtgaggccaggtaccttgtgggcagtgatgtcattcggtgcgactcctaagatgtctccagagatgggagagctcacccaaaccaggttgcagaagatctggattccacacagcagcggcagcagcaggctgcaacggagaaggggctcatccataccccagtttaccaattcccccacaatggtgatcatggtgggtttaccagctcgaggcaagacctatatctccacaaagctcacacgatatctcaactggataggaacaccaactaaagtgtttaatttaggccagtatcgacgagaggcagtgagctacaagaactatgaattctttcttccagacaacatggaagccctgcaaatcaggaagcagtgcgccctggcagccctgaaggatgttcacaactatctcagccatgaggaaggtcatgttgcggtttttgatgccaccaacactaccagagaacgacggtcactgatcctgcagtttgcaaaagaacatggttacaaggtgtttttcattgagtccatttgtaatgaccctggcataattgcagaaaacatcaggcaagtgaaacttggcagccctgattatatagactgtgaccgggaaaaggttctggaagactttctaaagagaattgagtgctatgaggtcaactaccaacccttggatgaggaactggacagccacctgtcctacatcaagatcttcgacgtgggcacacgctacatggtgaaccgagtgcaggatcacatccagagccgcacagtctactacctcatgaatatccatgtcacacctcgctccatctacctttgccgacatggcgagagtgaactcaacatcagaggccgcatcggaggtgactctggcctctcagttcgcggcaagcagtatgcctatgccctggccaacttcattcagtcccagggcatcagctccctgaaggtgtggaccagtcgcatgaagaggaccatccagacagctgaggccctgggtgtcccctatgagcagtggaaggccctgaatgagattgatgcgggtgtctgtgaggagatgacctatgaagaaatccaggaacattaccctgaagaatttgcactgcgagaccaagataaatatcgctaccgctatcccaagggagagtcctatgaggatctggttcagcgtctggagccagtgataatggagctagaacgacaggagaatgtactggtgatctgccaccaggctgtcatgcggtgcctcctggcctatttcctggataaaagttcagatgagcttccatatctcaagtgccctctgcacacagtgctcaaactcactcctgtggcttatggctgcaaagtggaatccatctacctgaatgtggaggccgtgaacacacaccgggagaagcctgagaatgtggacatcacccgggaacctgaggaagccctggatactgtcccagcccactactgagccctttccaagaagtcaaactgcctgtgtcctcatcgccttccacctttaggaaatgctatctttgctcttctcctactctgccttggcctcactgaggcaccccacttccagtgaagaagtcctccgcaactcccaaacaagcctcgcttgctggccgcaaccaaggagctatctagctctggaggaaactttctttcttaattcctattctctgacgaataaagacttactgcctacaagagg

SEQ ID NO: 66 - protein, 471 aa, Homo sapiens
MSPEMGELTQTRLQKIWIPHSSGSSRLQRRRGSSIPQFTNSPTMVIMVGLPARGKTYISTKLTRYLNWIGTPTKVFNLGQYRREAVSYKNYEFFLPDNMEALQIRKQCALAALKDVHNYLSHEEGHVAVFDATNTTRERRSLILQFAKEHGYKVFFIESICNDPGIIAENIRQVKLGSPDYIDCDREKVLEDFLKRIECYEVNYQPLDEELDSHLSYIKIFDVGTRYMVNRVQDHIQSRTVYYLMNIHVTPRSIYLCRHGESELNIRGRIGGDSGLSVRGKQYAYALANFIQSQGISSLKVWTSRMKRTIQTAEALGVPYEQWKALNEIDAGVCEEMTYEEIQEHYPEEFALRDQDKYRYRYPKGESYEDLVQRLEPVIMELERQENVLVICHQAVMRCLLAYFLDKSSDELPYLKCPLHTVLKLTPVAYGCKVESIYLNVEAVNTHREKPENVDITREPEEALDTVPAHY

SEQ ID NO: 67 - DNA, 2234 bp, Homo sapiens
ccccctccgctctgctgcgccgcccggctgggccccgaggccgctccgactgctatgtgaccgcgaggctgcgggaggaaggggacagggaagaagaggctctcccgcgggagcccttgaggaccaagtttgcggccacttctgcaggcgtcccttcttagctctcgcctgcccctttctgcagcctaggcggcccaggttctcttctcttcctcgcgcgcccagccgcctcggttcccggcgaccatggtgacgatggaggagctgcgggagatggactgcagtgtgctcaaaaggctgatgaaccgggacgagaatggcggcggcgcgggcggcagcggcagccacggcaccctggggctgccgagcggcggcaagtgcctgctgctggactgcagaccgttcctggcgcacagcgcgggctacatcctaggttcggtcaacgtgcgctgtaacaccatcgtgcggcggcgggctaagggctccgtgagcctggagcagatcctgcccgccgaggaggaggtacgcgcccgcttgcgctccggcctctactcggcggtcatcgtctacgacgagcgcagcccgcgcgccgagagcctccgcgaggacagcaccgtgtcgctggtggtgcaggcgctgcgccgcaacgccgagcgcaccgacatctgcctgctcaaaggcggctatgagaggttttcctccgagtacccagaattctgttctaaaaccaaggccctggcagccatcccacccccggttccccccagcgccacagagcccttggacctgggctgcagctcctgtgggaccccactacacgaccaggggggtcctgtggagatccttcccttcctctacctcggcagtgcctaccatgctgcccggagagacatgctggacgccctgggcatcacggctctgttgaatgtctcctcggactgcccaaaccactttgaaggacactatcagtacaagtgcatcccagtggaagataaccacaaggccgacatcagctcctggttcatggaagccatagagtacatcgatgccgtgaaggactgccgtgggcgcgtgctggtgcactgccaggcgggcatctcgcggtcggccaccatctgcctggcctacctgatgatgaagaaacgggtgaggctggaggaggccttcgagttcgttaagcagcgccgcagcatcatctcgcccaacttcagcttcatggggcagctgctgcagttcgagtcccaggtgctggccacgtcctgtgctgcggaggctgctagcccctcgggacccctgcgggagcggggcaagacccccgccacccccacctcgcagttcgtcttcagctttccggtctccgtgggcgtgcactcggcccccagcagcctgccctacctgcacagccccatcaccacctctcccagctgttagagccgccctgggggccccagaaccagagctggctcccagcaagggtaggacgggccgcatgcggcagaaagttgggactgagcagctgggagcaggcgaccgagctccttccccatcatttctccttggccaacgacgaggccagccagaatggcaataaggactccgaatacataataaaagcaaacagaacactccaacttagagcaataaccggtgccgcagcagccagggaagaccttggtttggtttatgtgtcagtttcacttttccgatagaaatttcttacctcatttttttaagcagtaaggcttgaagtgatgaaacccacagatcctagcaaatgtgcccaaccagctttactaaagggggaggaagggagggcaaagggatgagaagacaagtttcccagaagtgcctggttctgggtacttgtccctttgttgtcgttgttgtagttaaaggaatttcatttttaaaagaaatcttcgaaggtgtggttttcatttctcagtcaccaacagatgaataattatgcttaataataaagtatttattaagactttcttcagagtatgaaagtacaaaaagtctagttacagtggatttagaatatatttatgttgatgtcaaacagctgagcaccgtagcatgcagatgtcaaggcagttaggaagtaaatggtgtcttgtagatatgtgcaaggtagcatgatgagcaacttgagtttgttgccactgagaagcaggcgggttgggtgggaggaggaagaaagggaagaattaggtttgaattgcttttta

SEQ ID NO: 68 - protein, 394 aa, Homo sapiens
MVTMEELREMDCSVLKRLMNRDENGGGAGGSGSHGTLGLPSGGKCLLLDCRPFLAHSAGYILGSVNVRCNTIVRRRAKGSVSLEQILPAEEEVRARLRSGLYSAVIVYDERSPRAESLREDSTVSLVVQALRRNAERTDICLLKGGYERFSSEYPEFCSKTKALAAIPPPVPPSATEPLDLGCSSCGTPLHDQGGPVEILPFLYLGSAYHAARRDMLDALGITALLNVSSDCPNHFEGHYQYKCIPVEDNHKADISSWFMEAIEYIDAVKDCRGRVLVHCQAGISRSATICLAYLMMKKRVRLEEAFEFVKQRRSIISPNFSFMGQLLQFESQVLATSCAAEAASPSGPLRERGKTPATPTSQFVFSFPVSVGVHSAPSSLPYLHSPITTSPSC

SEQ ID NO: 69 - DNA, 776 bp, Homo sapiens
aaggacacgggcagcagacagtggtcagtcctttcttggctctgctgacactcgagcccacattccgtcacctgctcagaatcatgcaggtctccactgctgcccttgctgtcctcctctgcaccatggctctctgcaaccagttctctgcatcacttgctgctgacacgccgaccgcctgctgcttcagctacacctcccggcagattccacagaatttcatagctgactactttgagacgagcagccagtgctccaagcccggtgtcatcttcctaaccaagcgaagccggcaggtctgtgctgaccccagtgaggagtgggtccagaaatatgtcagcgacctagagctgagtgcctgaggggtccagaagcttcgaggcccagcgacctcggtgggccagtggggaggagcaggagcctgagccttgggaaacatgcgtgtgacctccacagctacctcttctatggactggttgttgccaaacagccacactgtgggactcttcttaacttaaattttaatttatttatactatttagtttttgtaatttattttcgatttcacagtgtgtttgtgattgtttgctctgagagttcccctgtcccctcccccttccctcacaccgcgtctggtgacaaccgagtggctgtcatcagcctgtgtaggcagtcatggcaccaaagccaccagactgacaaatgtgtatcggatgcttttgttcagggctgtgatcggcctggggaaataataaagcacgctcttttaaaaggt

SEQ ID NO: 70 - protein, 92 aa, Homo sapiens
MQVSTAALAVLLCTMALCNQFSASLAADTPTACCFSYTSRQIPQNFIADYFETSSQCSKPGVIFLTKRSRQVCADPSEEWVQKYVSDLELSA

SEQ ID NO: 71 - DNA, 2753 bp, Homo sapiens
gtcgggggcagcagcaagatgcgaagcgagccgtacagatcccgggctctccgaacgcaacttcgccctgcttgagcgaggctgcggtttccgaggccctctccagccaaggaaaagctacacaaaaagcctggatcactcatcgaaccacccctgaagccagtgaaggctctctcgcctcgccctctagcgttcgtctggagtagcgccaccccggcttcctggggacacagggttggcaccatggggcccaccagcgtcccgctggtcaaggcccaccgcagctcggtctctgactacgtcaactatgatatcatcgtccggcattacaactacacgggaaagctgaatatcagcgcggacaaggagaacagcattaaactgacctcggtggtgttcattctcatctgctgctttatcatcctggagaacatctttgtcttgctgaccatttggaaaaccaagaaattccaccgacccatgtactattttattggcaatctggccctctcagacctgttggcaggagtagcctacacagctaacctgctcttgtctggggccaccacctacaagctcactcccgcccagtggtttctgcgggaagggagtatgtttgtggccctgtcagcctccgtgttcagtctcctcgccatcgccattgagcgctatatcacaatgctgaaaatgaaactccacaacgggagcaataacttccgcctcttcctgctaatcagcgcctgctgggtcatctccctcatcctgggtggcctgcctatcatgggctggaactgcatcagtgcgctgtccagctgctccaccgtgctgccgctctaccacaagcactatatcctcttctgcaccacggtcttcactctgcttctgctctccatcgtcattctgtactgcagaatctactccttggtcaggactcggagccgccgcctgacgttccgcaagaacatttccaaggccagccgcagctctgagaagtcgctggcgctgctcaagaccgtaattatcgtcctgagcgtcttcatcgcctgctgggcaccgctcttcatcctgctcctgctggatgtgggctgcaaggtgaagacctgtgacatcctcttcagagcggagtacttcctggtgttagctgtgctcaactccggcaccaaccccatcatttacactctgaccaacaaggagatgcgtcgggccttcatccggatcatgtcctgctgcaagtgcccgagcggagactctgctggcaaattcaagcgacccatcatcgccggcatggaattcagccgcagcaaatcggacaattcctcccacccccagaaagacgaaggggacaacccagagaccattatgtcttctggaaacgtcaactcttcttcctagaactggaagctgtccacccaccggaagcgctctttacttggtcgctggccaccccagtgtttggaaaaaaatctctgggcttcgactgctgccagggaggagctgctgcaagccagagggaggaagggggagaatacgaacagcctggtggtgtcgggtgttggtgggtagagttagttcctgtgaacaatgcactgggaagggtggagatcaggtcccggcctggaatatatattctacccccctggagctttgattttgcactgagccaaaggtctagcattgtcaagctcctaaagggttcatttggcccctcctcaaagactaatgtccccatgtgaaagcgtctctttgtctggagctttgaggagatgttttccttcactttagtttcaaacccaagtgagtgtgtgcacttctgcttctttagggatgccctgtacatcccacaccccaccctcccttcccttcatacccctcctcaacgttcttttactttatactttaactacctgagagttatcagagctggggttgtggaatgatcgatcatctatagcaaataggctatgttgagtacgtaggctgtgggaagatgaagatggtttggaggtgtaaaacaatgtccttcgctgaggccaaagtttccatgtaagcgggatccgttttttggaatttggttgaagtcactttgatttctttaaaaaacatcttttcaatgaaatgtgttaccatttcatatccattgaagccgaaatctgcataaggaagcccactttatctaaatgatattagccaggatccttggtgtcctaggagaaacagacaagcaaaacaaagtgaaaaccgaatggattaacttttgcaaaccaagggagatttcttagcaaatgagtctaacaaatatgacatccgtctttcccacttttgttgatgtttatttcagaatcttgtgtgattcatttcaagcaacaacatgttgtattttgttgtgttaaaagtacttttcttgatttttgaatgtatttgtttcaggaagaagtcattttatggatttttctaacccgtgttaacttttctagaatccaccctcttgtgcccttaagcattactttaactggtagggaacgccagaacttttaagtccagctattcattagatagtaattgaagatatgtataaatattacaaagaataaaaatatattactgtctctttagtatggttttcagtgcaattaaaccgagagatgtcttgtttttttaaaaagaatagtatttaataggtttctgacttttgtggatcattttgcacatagctttatcaacttttaaacattaataaactgatttttttaaag

SEQ ID NO: 72 - protein, 382 aa, Homo sapiens
MGPTSVPLVKAHRSSVSDYVNYDIIVRHYNYTGKLNISADKENSIKLTSVVFILICCFIILENIFVLLTIWKTKKFHRPMYYFIGNLALSDLLAGVAYTANLLLSGATTYKLTPAQWFLREGSMFVALSASVFSLLAIAIERYITMLKMKLHNGSNNFRLFLLISACWVISLILGGLPIMGWNCISALSSCSTVLPLYHKHYILFCTTVFTLLLLSIVILYCRIYSLVRTRSRRLTFRKNISKASRSSEKSLALLKTVIIVLSVFIACWAPLFILLLLDVGCKVKTCDILFRAEYFLVLAVLNSGTNPIIYTLTNKEMRRAFIRIMSCCKCPSGDSAGKFKRPIIAGMEFSRSKSDNSSHPQKDEGDNPETIMSSGNVNSSS

SEQ ID NO: 73 - DNA, 1022 bp, Homo sapiens
acatacatcgggtgtgagcacggaatagctgggcggcggctggtccacagacactgggagacagaaggggccggtccccaggcgcccgtgctgcagcagtgggatccactggggagaggctggagaccaccggtgtgaaggcctcgcgggtaaattcactgcagctggacttgccaaagatgacctgggagagtggattcatgagggacccatgggggatgccctaccccccttccctggggcacagccactgaccggcagagcctggctggggtcctcgcctgtcatgtactgcactcgcacggcaaggttgcgcacggagccctggcggctgctgaagttgaggctgtgcagatggaggaggatattctgcagctgcaggcagaggccacagctgaggtgctgggggaggtggcccaggcacagaaggtgctacgggacagcgtgcagcggctagaagtccagctgaggagcgcctggctgggccctgcctaccgagaatttgaggtcttaaaggctcacgctgacaagcagagccacatcctatgggccctcacaggccacgtgcagcggcagaggcgggagatggtggcacagcagcatcggctgcgacagatccaggagagactccacacagcggcgctcccagcctgaatctgcctggatggaactgaggaccaatcatgctgcaaggaacacttccacgccccgtgaggcccctgtgcagggaggagctgcctgttcactgggatcagccagggcgccgggccccacttctgagcacagagcagagacagacgcaggcggggacaaaggcagaggatgtagccccattggggaggggtggaggaaggacatgtaccctttcatgcctacacacccctcattaaagcagagtcgtggcatctcaaaaaaaaaaaaaaaaaaaaaaaaaaaaaaaaaaaaaaaaaaaaaaaaaaaaaaaaaaaaaaaaaaaaaaaaaaaaaaaaaaaagggccaaaaaaaaaaaaaaaaaaaaaaaa

SEQ ID NO: 74 - DNA, 1743 bp, Homo sapiens
cagtatccctcctgacaaaactaacaaaaatcctgttagccaaataatcagccacattcatatttaccgtcaaagtttttatcctcattttacagcagtggagagcgattgccccgggtcccacgttaggaagagagagaactgggatttgcacccaggcaatctggggacagagctgtgatcacaactccatgagtcagggccgagccagccccttcaccaccagccggccgcgccccgggaaggaagtttgtggcggaggaggttcgtacgggaggagggggaggcgcccacgcatctggggctgactcgctctttcgcaaaacgtctgggaggagtccctggggccacaaaactgcctccttcctgaggccagaaggagagaagacgtgcagggaccccgcgcacaggagctgccctcgcgacatgggtcacccgccgctgctgccgctgctgctgctgctccacacctgcgtcccagcctcttggggcctgcggtgcatgcagtgtaagaccaacggggattgccgtgtggaagagtgcgccctgggacaggacctctgcaggaccacgatcgtgcgcttgtgggaagaaggagaagagctggagctggtggagaaaagctgtacccactcagagaagaccaacaggaccctgagctatcggactggcttgaagatcaccagccttaccgaggttgtgtgtgggttagacttgtgcaaccagggcaactctggccgggctgtcacctattcccgaagccgttacctcgaatgcatttcctgtggctcatcagacatgagctgtgagaggggccggcaccagagcctgcagtgccgcagccctgaagaacagtgcctggatgtggtgacccactggatccaggaaggtgaagaagggcgtccaaaggatgaccgccacctccgtggctgtggctaccttcccggctgcccgggctccaatggtttccacaacaacgacaccttccacttcctgaaatgctgcaacaccaccaaatgcaacgagggcccaatcctggagcttgaaaatctgccgcagaatggccgccagtgttacagctgcaaggggaacagcacccatggatgctcctctgaagagactttcctcattgactgccgaggccccatgaatcaatgtctggtagccaccggcactcacgaaccgaaaaaccaaagctatatggtaagaggctgtgcaaccgcctcaatgtgccaacatgcccacctgggtgacgccttcagcatgaaccacattgatgtctcctgctgtactaaaagtggctgtaaccacccagacctggatgtccagtaccgcagtggggctgctcctcagcctggccctgcccatctcagcctcaccatcaccctgctaatgactgccagactgtggggaggcactctcctctggacctaaacctgaaatccccctctctgccctggctggatccgggggacccctttgcccttccctcggctcccagccctacagacttgctgtgtgacctcaggccagtgtgccgacctctctgggcctcagttttcccagctatgaaaacagctatctcacaaagttgtgtgaagcagaagagaaaagctggaggaaggccgtgggcaatgggagagctcttgttattattaatattgttgccgctgttgtgttgttgttattaattaatattcatattatttattttatacttacataaagattttgtaccagtgg

SEQ ID NO: 75 - protein, 335 aa, Homo sapiens
MGHPPLLPLLLLLHTCVPASWGLRCMQCKTNGDCRVEECALGQDLCRTTIVRLWEEGEELELVEKSCTHSEKTNRTLSYRTGLKITSLTEVVCGLDLCNQGNSGRAVTYSRSRYLECISCGSSDMSCERGRHQSLQCRSPEEQCLDVVTHWIQEGEEGRPKDDRHLRGCGYLPGCPGSNGFHNNDTFHFLKCCNTTKCNEGPILELENLPQNGRQCYSCKGNSTHGCSSEETFLIDCRGPMNQCLVATGTHEPKNQSYMVRGCATASMCQHAHLGDAFSMNHIDVSCCTKSGCNHPDLDVQYRSGAAPQPGPAHLSLTITLLMTARLWGGTLLWT

SEQ ID NO: 76 - DNA, 2776 bp, Homo sapiens
ttgttggttgaaatgtaagtcttttcctaaagttttaatcagaggtagccatcactaagacttaagccacctgtggttctcttaagtttcactgaagccagaagaaggaaattaccacaacttgtattatactaattatcttcattattaacgatcatattagagccactgacatgtcccaaattatattaaaataaaaacctgcattgctctgacatgaagctcaattcaatgtaataaacaaattaggtattaaacgttataatttaaaaaacttctacgatatcaccagaaatcctggtgaaatttaattttttcccttttttaggtcagtggccggtgggatagatttttctaaactttttcctgctatgattttgaaggcaaatacatagagcacagttcagtaaaagagtttccaagccttttcactggagcatttacattttgattctgttgtcatttgaaatagaacatctgcagggagataatgagcttaagttacaattgactttggaaaaaataacagacttttgtgtttcctcaatcatgtagtcaatagggaattctccagttatgtgaacaatcacagtcttttaagacaatacttagatctaaatgcaaaatctagcatgcagaggctttttaaggattttagactgtctccaaagaacttggctcgagatggattacacagatttacaaatgcagttttcaactgtgacatcaaactgatcttatctcatttcatgttactcaactgtgcccgtaggaccccaagaggtggaatattttgtctacattattttaaacatctatagatatgtgtggatgtgtgaataatgcctaaatttctcatcttttaactagtttgtttttatcatacagacacagcctgttcataagaccagctttagacaacttagaactcaatgctctgaagattcagggaggccaacatagaaaaagatcttccaaaatatggggtggtccaacccatctcatccctctctagctggaaaactatagagaaagggctgtgactgccttgaccactgcctggcacaatgtaggcactcggtatttgttggatgaataagtggctgaatcagtgaatagacaaaaaacatggattttgccttctgaaaatctcacaaattttaccagcttatattatactgattgtcactatgatttctaagtttcatggttagaaccatattttgagtctatataaaaactttttagatgtatgaatttgaaatttcattaattactttaaaggcttcaaagatttgaggacatttgaacagtggataaggaacaatgtttggggatacagagtttttatgctgagttgaactcaacctttctagttctttatacaagcttcctgaattatctggcaggaatgattcaaacataaaaccccatttttaaattttataaaagatattttcatttttatataaaaagatcatcaggcatattatcccctttgatatttattaacaaccattctctaagaaattcataataattggagacaccatccctaaatctcttcctattttcaaaagatcatttatcctttcatatttgattcctggaatacactaagctgaagtttaatcacatacttactcagatgtttccatgtgtttattcatttaacaaatacttattgtgtacctagtgcatgctaggttctggaaatgcaacagcaaacaagacagacacaatccctcttggaggttactgtgagaaactctccaacatttactctagcaataactagagagtttactgtccattgggtcctcttcacacctgatagaaaaaataaaattcctcaccccatggaagtcatgaaatactccctgcacagatttgtgtgagcttgcttttctaaaaccactgctcaaaaatgctgcttagtttatctataaaccacggttataccccagtgaggtaggtaagtaatttattcataactaacatttatgcagctctattttctagccactgtgctgagtgcttttatggactatctcaccaaatcccttcaacaactctattcagtaggttccattttatagatacaaaaactgaagcacagatatattatgtgacttgtccaaggtcacacagctagcaggtggaactgggatttgaacccatgcaatctgacacaagagttcatgtgtctaatcattatctgtggtttgggggacaccattacattaaaatgaatgagtcactaagtgagtcactgtacataaaatacttggaaattgtaaagtaatatacaaaagatattaacaaagtattattcatggtaaacaaatcacatcagtagcaaacacagggtgggaactcaatcttatgttatagcttactcttaagaagaatatgtgaagccaggaacatggttaaaggtacagaccagaaaatttctgacatgtgataaatatttcagtgacttttcagatttatttcttgttagccgctgtgtctatttggtgctacaaaaactgaaagaaacaaaatccctgatgtaagggcttataataaatacacagtttggagatgaactaaggatattttacatttacgggaaaggattggacaatagtatcattttctcaccatttgcatgtacatcatgtctttccaattgttttcattatttttctgaaagagctgcaaaattgtgcagtggttcaacctgaccaaagtggtattatgctgctgggaagtaaaagtattagacacc

SEQ ID NO: 77 - DNA, 3362 bp, Homo sapiens
agactccctgtctttgcggtttgggagatgatgagaaaccacagaattgctagtagtttatgtggagatcaggtcttctccaagaaaaaaaaaaagaaaaaaaaaaacaacatggctgcaaaggagaaactggaggcagtgttaaatgtggccctgagggtgccaagcatcatgctgttggatgtcctgtacagatgggatgtcagctcctttttccagcagatccaaagaagtagccttagtaataaccctcttttccagtataagtatttggctcttaatatgcattatgtaggttatatcttaagtgtggtgctgctaacattgcccaggcagcatctggttcagctttatctatattttttgactgctctgctcctctatgctggacatcaaatttccagggactatgttcggagtgaactggagtttgcctatgagggaccaatgtatttagaacctctctctatgaatcggtttaccacagccttaataggtcagttggtggtgtgtactttatgctcctgtgtcatgaaaacaaagcagatttggctgttttcagctcacatgcttcctctgctagcacgactctgccttgttcctttggagacaattgttatcatcaataaatttgctatgatttttactggattggaagttctctattttcttgggtctaatcttttggtaccttataaccttgctaaatctgcatacagagaattggttcaggtagtggaggtatatggccttctcgccttgggaatgtccctgtggaatcaactggtagtccctgttcttttcatggttttctggctcgtcttatttgctcttcagatttactcctatttcagtactcgagatcagcctgcatcacgtgagaggcttcttttcctttttctgacaagtattgcggaatgctgcagcactccttactctcttttgggtttggtcttcacggtttcttttgttgccttgggtgttctcacactctgcaagttttacttgcagggttatcgagctttcatgaatgatcctgccatgaatcggggcatgacagaaggagtaacgctgttaatcctggcagtgcagactgggctgatagaactgcaggttgttcatcgggcattcttgctcagtattatccttttcattgtcgtagcttctatcctacagtctatgttagaaattgcagatcctattgttttggcactgggagcatctagagacaagagcttgtggaaacacttccgtgctgtaagcctttgtttatttttattggtattccctgcttatatggcttatatgatttgccagtttttccacatggatttttggcttcttatcattatttccagcagcattcttacctctcttcaggttctgggaacactttttatttatgtcttatttatggttgaggaattcagaaaagagccagtggaaaacatggatgatgtcatctactatgtgaatggcacttaccgcctgctggagtttcttgtggccctctgtgtggtggcctatggcgtctcagagaccatctttggagaatggacagtgatgggctcaatgatcatcttcattcattcctactataacgtgtggcttcgggcccagctggggtggaagagctttcttctccgcagggatgctgtgaataagattaaatcgttacccattgctacgaaagagcagcttgagaaacacaatgatatttgtgccatctgttatcaggacatgaaatctgctgtgatcacgccttgcagtcattttttccatgcaggctgtcttaagaaatggctgtatgtccaggagacctgccctctgtgccactgccatctgaaaaactcctcccagcttccaggattaggaactgagccagttctacagcctcatgctggagctgagcaaaacgtcatgtttcaggaaggtactgaacccccaggccaggagcatactccagggaccaggatacaggaaggttccagggacaataatgagtacattgccagacgaccagataaccaggaaggggcttttgaccccaaagaatatcctcacagtgcgaaagatgaagcacatcctgttgaatcagcctagaggagaagcagcaggaatgatgctttgatactctggaggagaagttaactcaagatggaattcatgttctgatttgaggaatgaaaatgagatgatcaggcaggaaactgacattccaaggatctaatccaggaagtactctcagtggggaccacctgctttcatcccctgacattgtgggagaaattttgcaatgtatgctaatcaaaatgtatttatatgttctctgctgatgttttatagaggtttgtgaagaaaattcaacctcagcaacttcagaaactgcccctgatacgtgtgagagagaaataaaatcagattttgagtgttgaagggactgaggaagtgaggataaagagcatgaggacagcatggaaagaaggaggcagaagtggaactgaactttcactctccatgggacagatcaatctcattatcaagtctgaatagcaaccagccctctcctccaccccgtttctcctcagttaattggagctcagtcaggtgattattgagtcttgtacagcactgaaatgaaatcaaagatgaagaagcattgattgtattcgaagattgaagcacgctcatactttgtatgtgctttagggaaggggtgggtgggcacttgggccttgcgggtgcattcatgtaatctgagactcttgaactttatgacggagtcttcaatattttgatgtatatgaaacttttgttaaatatgttgtatacttcgctggctgtgtgaagtaaactaaaactctgatgaacactttggagtctgctttagtgaaggagaccaaagtgggaagggctttagggcactgatagaggccctgggtgtacttttcaatcctgtgtaatgtttaattcttgcaactgaatcaaaacagtgttaaattatggcaatatttgcactttgggaatgagtacataactgtatgatcacactctgcaaatgccacttttaaagctgttaatagactttgcaccttttctttgacaaggatgtgtcatatttaaatttttacattcatcatggctacaggtagaactggggaggggggaatgtaattttttatgggaattttgatatgaaaagaaactagtcatttatttatacaataggcttggctcaaaaagtgtttttcagacctcggtattcctaatgtgggatgtgactttattttatttttagtagcaaatttggatgtagactgacagacatagctgaatgtcttaataaatttaaatttgaagat

SEQ ID NO: 78 - protein, 691 aa, Homo sapiens
MMRNHRIASSLCGDQVFSKKKKKKKKNNMAAKEKLEAVLNVALRVPSIMLLDVLYRWDVSSFFQQIQRSSLSNNPLFQYKYLALNMHYVGYILSVVLLTLPRQHLVQLYLYFLTALLLYAGHQISRDYVRSELEFAYEGPMYLEPLSMNRFTTALIGQLVVCTLCSCVMKTKQIWLFSAHMLPLLARLCLVPLETIVIINKFAMIFTGLEVLYFLGSNLLVPYNLAKSAYRELVQVVEVYGLLALGMSLWNQLVVPVLFMVFWLVLFALQIYSYFSTRDQPASRERLLFLFLTSIAECCSTPYSLLGLVFTVSFVALGVLTLCKFYLQGYRAFMNDPAMNRGMTEGVTLLILAVQTGLIELQVVHRAFLLSIILFIVVASILQSMLEIADPIVLALGASRDKSLWKHFRAVSLCLFLLVFPAYMAYMICQFFHMDFWLLIIISSSILTSLQVLGTLFIYVLFMVEEFRKEPVENMDDVIYYVNGTYRLLEFLVALCVVAYGVSETIFGEWTVMGSMIIFIHSYYNVWLRAQLGWKSFLLRRDAVNKIKSLPIATKEQLEKHNDICAICYQDMKSAVITPCSHFFHAGCLKKWLYVQETCPLCHCHLKNSSQLPGLGTEPVLQPHAGAEQNVMFQEGTEPPGQEHTPGTRIQEGSRDNNEYIARRPDNQEGAFDPKEYPHSAKDEAHPVESA

SEQ ID NO: 79 - DNA, 1956 bp, Homo sapiens
ggtggccctgagcgccggcgacacctttcctggactataaattgagcacctgggatgggtagggggccaacgcagtcaccgccgtccgcagtcacagtccagccactgaccgcagcagcgcccttgcgtagcagccgcttgcagcgagaacactgaattgccaacgagcaggagagtctcaaggcgcaagaggaggccagggctcgacccacagagcaccctcagccatcgcgagtttccgggcgccaaagccaggagaagccgcccatcccgcagggccggtctgccagcgagacgagagttggcgagggcggaggagtgccgggaatcccgccacaccggctatagccaggcccccagcgcgggccttggagagcgcgtgaaggcgggcatccccttgacccggccgaccatccccgtgcccctgcgtccctgcgctccaacgtccgcgcggccaccatgatgcaaatctgcgacacctacaaccagaagcactcgctctttaacgccatgaatcgcttcattggcgccgtgaacaacatggaccagacggtgatggtgcccagcttgctgcgcgacgtgcccctggctgaccccgggttagacaacgatgttggcgtggaggtaggcggcagtggcggctgcctggaggagcgcacgcccccagtccccgactcgggaagcgccaatggcagctttttcgcgccctctcgggacatgtacagccactacgtgcttctcaagtccatccgcaacgacatcgagtggggggtcctgcaccagccgcctccaccggctgggagcgaggagggcagtgcctggaagtccaaggacatcctggtggacctgggccacttggagggtgcggacgccggcgaagaagacctggaacagcagttccactaccacctgcgcgggctgcacactgtgctctcgaaactcacgcgcaaagccaacatcctcactaacagatacaagcaggagatcggcttcggcaattggggccactgaggcgtggcgcccgtggctgcccagcaccttcttcgacccatctcaccctctctcattcctcaaagctttttttttttttcctggctggggggcgggaagggcagactgcaaactggggggctgcgtacgtgcaggaggcgcggtggggctgcgtggaggagggggccacgtgtgagagagaagaaaatggtggccggagatgggagggcccaaggaacctcctgggagggggcctgcattctatgttggtgggaatgggactgggctgacgccctgcattcagcctgtgcctttcctggggtttcttttctgttcttttcggaggagagggcccgagaaggggccataccagggcgcggcgctgggttgccacacttgggaaagcagcccggagctgggtgctggggaaggcggggcgcgtagcctcccgccgccctgcggttgggccggtggaggcccaggcgttgctaggattgcatcagttttcctgtttgcactatttctttttgtaacattggccctgtgtgaagtatttcgaatctcctccttgctctgaaacttcagcgattccattgtgataagcgcacaaacagcactgtctgtcggtaatcggtactactttattaatgattttctgttacactgtatagtagtcctatggcacccccaccccatccctttcgtgccactcccgtccccacccccaccccagtgtgtataagctggcatttcgccagcttgtacgtagcttgccactcagtgaaaataataacattattatgagaaagtggacttaaccgaaatggaaccaactgacattctatcgtgttgtacatagaatgatgaagggttccactgttgttgtatgtcttaaatttatttaaaactttttttaatccagatgtagactatattctaaaaaataaaaaagcaaatgtgtcaact

SEQ ID NO: 80 - protein, 183 aa, Homo sapiens
MMQICDTYNQKHSLFNAMNRFIGAVNNMDQTVMVPSLLRDVPLADPGLDNDVGVEVGGSGGCLEERTPPVPDSGSANGSFFAPSRDMYSHYVLLKSIRNDIEWGVLHQPPPPAGSEEGSAWKSKDILVDLGHLEGADAGEEDLEQQFHYHLRGLHTVLSKLTRKANILTNRYKQEIGFGNWGH

SEQ ID NO: 81 - DNA, 427 bp, Homo sapiens
gagtgttctccccagtgactgcccaatctttagtgtgttttgctgactcctggcgctctggcttttccataagaggcgtttttgcacgattgcagacaggaataaaaatgagctctgcctactgggccgtcctggttagcaaggtctacaccacctcaaaggcagctatggactaagagcccacaaagaatctcgggttatgagggaggggctggccgtggagacggctaaagcgcctcatttgtcttcccagcagggaagcgatgggacctcggcgggcagggaacgcgatctccacccgtctggaaaactctctgagttctggagcactgttcgcggccggactgagtggcatgtccgcattttccccattccagggtagggcagactgaggtaggagagcagagccccgaggccgactccccgg

SEQ ID NO: 82 - DNA, 2365 bp, Homo sapiens
catcctgacatttcccttgtacctgctgaactttctgggcttgtggagctggatatgcaaaaaatggttcccctacttcttggtgaggttcactgtgatatacaacgaacagatggcaagcaagaagcgggagctcttcagtaacctgcaggagtttgcgggcccctccgggaaactctccctgctggaagtgggctgtggcacgggggccaacttcaagttctacccacctgggtgcagggtgacctgtattgaccccaaccccaactttgagaagtttttgatcaagagcattgcagagaaccgacacctgcagtttgagcgctttgtggtagctgccggggagaacatgcaccaggtggctgatggctctgtggatgtggtggtctgcaccctggtgctgtgctctgtgaagaaccaggagcggattctccgcgaggtgtgcagagtgctgagaccgggaggggctttctatttcatggagcatgtggcagctgagtgttcgacttggaattacttctggcaacaagtcctggatcctgcctggcaccttctgtttgatgggtgcaacctgaccagagagagctggaaggccctggagcgggccagcttctctaagctgaagctgcagcacatccaggccccactgtcctgggagttggtgcgccctcatatctatggatatgctgtgaaatagtgtgagctggcagttaagagctgaatggctcaaagaatttaaagcttcagttttacatttaaaatgctaagtgggagaagagaaaccttttttttggggggcggtttttttggtttgttgttggtttttttttttttttggcgggaagaaagagttttgctcttgttgcccaggctggagtgcagtgacgtgatctccgctcactgcaacctccacctcgcgggtttaagcgattcttctgcctcagcctccctagtagctgggattacaggtgcccaccaccatgcccagctaatttgtatttttagttgagacagggtttcactacgttggccaggctggtcttgaactcctgatctcaggcaatccacccgcctcagcctcccaaagtgctgggatgacaggcgtgagcaaccgcacccagcttaaggtttttttgttttgttttgagacggagttttcgctcttgttgcccaggctggagtgcaatgctgtgatctcagcttaccacaacctccacctcccgggttcaagtgattcacctgcctcagcctcctgagtagctggtattacaggcatgcgtcaccacgccggctaattttgtacttttagtagagatggtgtttccccacgttggtcagtctggtctcaaattcctgacctcaggtgatctgcctgcctcggcctcccaaagtgctgtgattacagacgtcagccaccatgcctggcctgaaaccttttttaggtaaagttgaattccatccttaaaagtttctgttatatcctatttagccattttctattgtctcccaaagaattcacatcaaaaaaacagctttgaactcccccttcaaaggaaacagtcgactttcataattagcatctaccattatccccaaatcttattttattcattgacttgaaattttttccaattgctttttttttttttttttaaggttaagagcagaggtttactaggccaaagaaagagaatagctctctgttgcagagaggggtcctggagaaatgggttaccccagttgtcttatttaaatggttacccatcagattttaattttatcttctctttgagagcttggtaataagaagcacttaaatcactccaaagaagactttaaaaagggagcagtgaaaaggtcttaataatttattgattgaattaagaaatactagctaattaagaatctgagtctaaacagcacagattttttctttctgcttttaaattgtgttttaaaaaaagagacagggggctgggcgtggtggctcacgcctgtaatcctagcactttgggaggccgaggcgggtggatcacgaggtaggagttaaagaccagcctggccaacatggcaaaaccctactaaagatacaaaaaaaaaaaaaaaaaaaaaaaaagtatatacacctaacaagagagccccaaaatgtatgaagcaaaaactgacagaactgaagggacaaatagacaattcaatagtaatatttggagacttcaatatcccactttcaataatgaataggacaactaagcaaaagattaacaagaacactgaagacctgaaaagcactataaacaaaactataagctaactagacagaacacacatctacagaacaaaaactccaaccaaaaaaaaaaaaaaa

SEQ ID NO: 83 - protein, 194 aa, Homo sapiens
MASKKRELFSNLQEFAGPSGKLSLLEVGCGTGANFKFYPPGCRVTCIDPNPNFEKFLIKSIAENRHLQFERFVVAAGENMHQVADGSVDVVVCTLVLCSVKNQERILREVCRVLRPGGAFYFMEHVAAECSTWNYFWQQVLDPAWHLLFDGCNLTRESWKALERASFSKLKLQHIQAPLSWELVRPHIYGYAVK

SEQ ID NO: 84 - DNA, 345 bp, Homo sapiens
ctgtgccttttaaggcagagggagatccctatggcgttagtcttcccaggccttaaagggcccttgtcttcactcacaaacctcttatctcttcttctccttcctctacattttaaagggggagagggaaaagtaaccgggagacaaattgagccacatattttcagacacttgttaccatattttaaaatctggcttcacatacacagagtctttgctatgcaccatgtactgttctaagcttcttaaaaatagaatctcaattattattttgcaggcaatactctatgcattcattagctaggacaacaatgcatttgcagtagtgagatttcgttaaaaaat

SEQ ID NO: 85 - DNA, 348 bp, Homo sapiens
aatacaatattagtaacagttctatcaatatgctaaaagctttacctcctcctactccttcatttttaatcaaaataggaccttccctgaaggattgggttatgagtacaatgaatttatgttggtctagtaggcaatattatgatttaaaagcatttctcaatggccaagtagataaggaattatatgaaattctacaaataccaagaggtatttcagtggtttacaaatattgtctaccttctgtatgcataaaatgtcatagattggtgccagtgaacacacacacgtatctcaggtgaggattggcctcagagaaattactggtacattctagaagacaaaaat

SEQ ID NO: 86 - DNA, 384 bp, Homo sapiens
aagagtcccaaatgtgaagtgtccattttcctataaaatcaggacatgttacccttccagtatggatatgaaatatcaacatgtgtcccactgttcagagttttattggggctccattacctagtcatgattgattgattgattgattgcccaggtggttgaactcagtctccaggttgaccgatactgtatgacgcagatgccctaccccagatcacatggttagtctttctggcatggccagcgttcacccggtgactctcagttatggtcgctaagctactagataaaaggattaggcccttattcaaactggttgaatgcttgtcaactgtcaccagagtccatccacagaataaatagaaatataagaggcaaacagac

SEQ ID NO: 87 - DNA, 440 bp, Homo sapiens
cgtgcctggctaatttttgcagttttagtagaggcagggtttcaccaccttggccaggctggtcttgaactcctgacctcatgatccacccaccttgtcctctcaaaatgcttacaggcatgaaccactgtgaccggccaggtcttttttttttttaaagagtctatcctaggcttggtacagatggcgcctgtggtaaatctgtgttaacatggtggtagaccatggagaatgggtagatggagcttctagactccaaaacaacaaaaccacagtgtgagtacactgtcccattaggttgactgaagtctgagtacattttctggcctgcgtgccgtgacttatccaacctgtgaactgattgtgatctgcttggtaacttggtttggtgtaaactgctcttaaccctttgctgatgaagaaaataatcatataagtgg

SEQ ID NO: 88 - DNA, 400 bp, Homo sapiens
gccactggacatttgcagagatgatcatatttacttccctcctcccaccactcttcctctcagtttcctttttactttatttttttctccctttcctggtaaaagtattagtatgtttaacatagtgttaggtggcagataggctgggaagagctagtattgtaaaggaacactgatgtgtactggagcctactagctctcaatccattgccccagacttggggagagagagcacattctctatccagagaaataagaatatgcagtttatggttgtcctgtggacattaactgactgtgaaggtaaggtatatccaaaggcagtgatatgcaggtgatagattcaccaggagatgatatgtgacttttcttgtattttaaagctctgcttcttttttag

SEQ ID NO: 89 - DNA, 344 bp, Homo sapiens
ctagcaccactccaaattgctgcttcaaaagaggtcagtcagcccctttcccttactcctcaaccatgacctggtctgtctggaaagaggatgagatacatggaccaatacatcctatttaaaagggagcttgctttctcactagggactttagactgcctgacacacaggaaatgactagaaaatctgaccagtggagcataactcattgctaaaatggcaggtttggacggctgcaggatgggagagcaggcttcctgaagaggcaggaaggggcagctggatgggaaaacagtgacttttgagtttttccttttagagtatggtgacgtaggccgtttatt

SEQ ID NO: 90 - DNA, 500 bp, Homo sapiens
accaagtttgattcaagcacattggttagtggaataaagcaagctgtgctgttatatagttgttggtctttcaaatgcctcagaaccagaataagacaaatgagggccagagtagaggttgctagagagctgtgtatattgttaggtactgatttggatgacagtgattgctataagcatttacaatacttcgtagtttagccacagttgtcccagatagctgcccacagctttaaaacatttttagggtgtctgttgccaccctagtcgttgacactgcatggagattatttctgtgacagcagtcttcaccagtgttaaggaggctgggagttgactctgtttccataaccatctaattaaatcaagtgcccataatggaatttgaataactgcctaactttgatatttaaacagaaactttgaggaaaaacaagtctctttttgtaaatatattctacttgttaagaaaaaataaagtagatcatgatgttcatctg

SEQ ID NO: 91 - DNA, 476 bp, Homo sapiens
gatcagtgtgtgctggtgattttattgccttttcctcttaattcaatcccactctttcccttcctcaagtcttgttcacccagcctcctgccccaggaatcacggggaagaagtctgggagaccacagaaaaaacaccaatgacactgagctattagagatcaaaactgggtcaggaagagttcatgttaagctttcacatgtgctaagaacgctgaaagatacacactgatttccatcttaatagtgccggataattgacaaatttgggacaagatgagggagaggtcaaaatccccctaacttcccaaagcaaaaaaacaggagtataatgatcagaaacgtaatagtagtgtcagaagcacacaatcagctgtggacggtgaaacatataatggttctatctgttctgagattgtaaaagcacaattattttcatcaggataaaaaataaaaatgaaaaactatgctgttgaa

SEQ ID NO: 92 - DNA, 439 bp, Homo sapiens
tcttcagcctggctcttgagctattgctgtattaattttaaatagggtgtgatagcataagctgatggaagcctgcagagatctcactttgaaatggtgatacattacatgggaaaagattagagaggtgttttatactgcacatcggtgaggcctaataagaaagtagaatagacgtaaaccattgttttcatcatcctttaagacagggatttcaaactcaagaaccaggcagctacctatttaaatgagtcaagtgggccctaatgtaagacaatgtgatgggtaaggattgattctgagggaacaggagcaggcatgccctctctaacagaggccgtcagttctgctgtacctgaaatgtgggcccattgttgctggcgattccacacttacatctaaaatttcctgattttttaagaatgacaataactaattt

SEQ ID NO: 93 - DNA, 520 bp, Homo sapiens
agagagtcagtgagggtgttagaggtagctgaaggagtggaagggagttagctagttgaaggaacactggaggactgtcggggatttgggggattaagtaaaaggtttataaggagagggtggaaagagtggttgatggctgatattctgatcatttatactagctactcagtctctactttttctagaagtccccagtaaccaaagcccaacacatttggtatcgttgattccttccattgtttgctttgctttgggttgcctaaggctgtggttatctctgagccacctaaattctcaccctcagctgaaatcactactgtctcttcatggctctcagtatatttcaatccttcttcaggaactggagcttttgagaataaggcaactttaggattgaccctaggacagaagctctgctctcaggatccttgtatgctcctgaatatgaattccatagtatgatatttggagctccccagatccttgtaggcatatgtgtgtgtgtgtatttgaggcc

SEQ ID NO: 94 - DNA, 557 bp, Homo sapiens
gcatgcactgtctcagccaacccgctccactacccggcagtacacattcgcacccctacttcacagaggaagaaacctggaaccagagggggcgtgcctgccaagctcacacagcaggaactgagccagaaacgcagattgggctggctctgaagccaagcctcttcttacttcacccggctgggctcctcatttttacgggtaacagtgaggctgggaaggggaacacagaccaggaagctcggtgagtgatggcagaacgatgcctgcaggcatggaactttttccgttatcacccaggcctgattcactggcctggcggagatgcttctaaggcatggtcgggggagagggccaacaactgtccctccttgagcaccagccccacccaagcaagcagacatttatcttttgggtctgtcctctctgttgcctttttacagccaacttttctagacctgttttgcttttgtaacttgaagatatttattctgggttttgtagcatttttattaatatggtgactttttaaaataaaaacaaacaaacgttgtcct

SEQ ID NO: 95 - DNA, 2160 bp, Homo sapiens
gcgtctgccagccggcttggctagcgcgcggcggccgtggctaaggctgctacgaagcgagcttgggaggagcagcggcctgcggggcagaggagcatcccgtctaccaggtcccaagcggccgtggcccgcgggtcatggccaaaggagaaggcgccgagagcggctccgcggcggggctgctacccaccagcatcctccaaagcactgaacgcccggcccaggtgaagaaagaaccgaaaaagaagaaacaacagttgtctgtttgcaacaagctttgctatgcacttgggggagccccctaccaggtgacgggctgtgccctgggtttcttccttcagatctacctattggatgtggctcaggtgggccctttctctgcctccatcatcctgtttgtgggccgagcctgggatgccatcacagaccccctggtgggcctctgcatcagcaaatccccctggacctgcctgggtcgccttatgccctggatcatcttctccacgcccctggccgtcattgcctacttcctcatctggttcgtgcccgacttcccacacggccagacctattggtacctgcttttctattgcctctttgaaacaatggtcacgtgtttccatgttccctactcggctctcaccatgttcatcagcaccgagcagactgagcgggattctgccaccgcctatcggatgactgtggaagtgctgggcacagtgctgggcacggcgatccagggacaaatcgtgggccaagcagacacgccttgtttccaggacctcaatagctctacagtagcttcacaaagtgccaaccatacacatggcaccacctcacacagggaaacgcaaaaggcatacctgctggcagcgggggtcattgtctgtatctatataatctgtgctgtcatcctgatcctgggcgtgcgggagcagagagaaccctatgaagcccagcagtctgagccaatcgcctacttccggggcctacggctggtcatgagccacggcccatacatcaaacttattactggcttcctcttcacctccttggctttcatgctggtggaggggaactttgtcttgttttgcacctacaccttgggcttccgcaatgaattccagaatctactcctggccatcatgctctcggccactttaaccattcccatctggcagtggttcttgacccggtttggcaagaagacagctgtatatgttgggatctcatcagcagtgccatttctcatcttggtggccctcatggagagtaacctcatcattacatatgcggtagctgtggcagctggcatcagtgtggcagctgccttcttactaccctggtccatgctgcctgatgtcattgacgacttccatctgaagcagccccacttccatggaaccgagcccatcttcttctccttctatgtcttcttcaccaagtttgcctctggagtgtcactgggcatttctaccctcagtctggactttgcagggtaccagacccgtggctgctcgcagccggaacgtgtcaagtttacactgaacatgctcgtgaccatggctcccatagttctcatcctgctgggcctgctgctcttcaaaatgtaccccattgatgaggagaggcggcggcagaataagaaggccctgcaggcactgagggacgaggccagcagctctggctgctcagaaacagactccacagagctggctagcatcctctagggcccgccacgttgcccgaagccaccatgcagaaggccacagaagggatcaggacctgtctgccggcttgctgagcagctggactgcaggtgctaggaagggaactgaagactcaaggaggtggcccaggacacttgctgtgctcactgtggggccggctgctctgtggcctcctgcctcccctctgcctgcctgtggggccaagccctggggctgccactgtgaatatgccaaggactgatcgggcctagcccggaacactaatgtagaaaccttttttttacagagcctaattaataacttaatgactgtgtacatagcaatgtgtgtgtatgtatatgtctgtgagctattaatgttattaattttcataaaagctggaaagcaaaaaaaaaaaaaaaaaaaaaaaaaaaaaaaaaaaaaaaaaaaa

SEQ ID NO: 96 - protein, 530 aa, Homo sapiens
MAKGEGAESGSAAGLLPTSILQSTERPAQVKKEPKKKKQQLSVCNKLCYALGGAPYQVTGCALGFFLQIYLLDVAQVGPFSASIILFVGRAWDAITDPLVGLCISKSPWTCLGRLMPWIIFSTPLAVIAYFLIWFVPDFPHGQTYWYLLFYCLFETMVTCFHVPYSALTMFISTEQTERDSATAYRMTVEVLGTVLGTAIQGQIVGQADTPCFQDLNSSTVASQSANHTHGTTSHRETQKAYLLAAGVIVCIYIICAVILILGVREQREPYEAQQSEPIAYFRGLRLVMSHGPYIKLITGFLFTSLAFMLVEGNFVLFCTYTLGFRNEFQNLLLAIMLSATLTIPIWQWFLTRFGKKTAVYVGISSAVPFLILVALMESNLIITYAVAVAAGISVAAAFLLPWSMLPDVIDDFHLKQPHFHGTEPIFFSFYVFFTKFASGVSLGISTLSLDFAGYQTRGCSQPERVKFTLNMLVTMAPIVLILLGLLLFKMYPIDEERRRQNKKALQALRDEASSSGCSETDSTELASIL

SEQ ID NO: 97 - DNA, 2952 bp, Homo sapiens
acaaactactccctgagatcctacaatccgcttcatttctaactgctaatggggccttgtatatggctttcttttgcattttaaggaagatacttggaaaattctactcatggactcctggctttggtgccgctctgccagcatcttatgtggccattctcattgaaagaaaaagcaggagagggctgctcacaatttatatggccaacttggtccttttgttttgcatcacagctgccatgtacatgttctttttcaggtgcaaggatggcttgaaaggatttacattttctgcacttaggttcattgtagggaaggaagaaattcccacacattctttttcaccagaggcagcatatgcaaaagtggaacaaaagagagagcaacatgaggaaaaacccagaagaatgaatatgattggtctagtcaggaaatttgtggattcaatatgcaaacatggaccaaggcatagatgttgcaaacattatgaagataattgcatctcttattgcattaaaggtttcatcagaatgtttagcgtggggtacttgatccagtgctgcctccgaatcccttctgcatttaggcatctgtttacacagccatctcggctactttctctcttctacaataaagaaaacttccagcttggagcttttcttggctcttttgttagtatatacaagggtactagttgcttcctgcgctggatcagaaacttagatgatgaactacatgctattatagctggatttttggcaggtacatcaatgatgttttataaaagcacaacaatttccatgtatttagcgtccaaattggtagagacaatgtatttcaaaggcattgaagcagggaaggttccctattttcctcatgcagatactatcatctattccatctctacagcaatttgcttccaggcagctgtcatggaagttcagactttgagaccatcttactggaagttccttttaagactcaccaagggcaaatttgctgtcatgaaccgaaaagtccttgatgtttttggtactggtgcatctaaacactttcaggatttcatccccaggttggatccaagatacacaactgtaacaccagagttgcccacagagttttcctgaagatgactgtaacttattaatgtgactaaatgtttcatcttgaagagttaattatgttgaacacaaaggagggggcccaagctcgaacttcagtgttatttcagttagagatactcttttcatttgttttgtttttcttatgaatcagaaattcagaagctttttaggaagatgttgcttaataattaagcttcctccatagccagaataagattctggatcactgtagtgactgacattatatattattgatcaaattatgtccacaagcaatattatataatctacgtagaagtgtaataacaaacaagagtacacttaaaattactttaaaagatgtctttagttcattccaatataattcttgattaaaattaggattatttctacattttaggatttacaaaggatcacgggtacatgggtttggtctatatatttttttaaagttttgaattggtatctgtagtagtggaatgttatagatttgaagtaactctccacggacagtgctgctttcgtgtagagcaatttaattggagaagtggccattcttacttcagggatgcaaagatgggtctcataccatttggataaatgtcgtggtatccatgctttttttcaactaataacatcatctctcttcatgaccagttaattgggctatttggcagcccagtgaacctatgtactaatggcaagttaggggcaaatggaaatggacacatccgttaaagttgaaatgtatgttttaatctttcacagaagtattacacttgaatatttaaaaacaaaacttttaaacttcctataggtttatgatgtttgttttcatttatatggacataatccttcatagctcagtttatatgccattgttgtattagaagggatcaaaatcctatggaacaaagtagtcttggcaagttggcagtttgtgtcctctcagctgtttaacttatgtaatggatgttttgcacctgaaaacactataaaaatccagtggttgtttaaaaagtccatttgtcactaattccattcaggttctccaaccttcttcttgaatatcattgtcaccatttatactgttagaataaagaggtgacaccataaagtcctgctgataatgagagtagttcaggacagctgtgattgaaatatggtcgctatttacagtttttcagggaaaagttatacttttctatgttaataaagagctgaagtggtctacagttaatgtgacatgtagggatgatgatatttttaaaatacattttgttgctaaaaagttgttaggccagtgcaaattatgcagtagaacttgtgttgcaaaaggaattataacccatactttaaaaatgcttaatccctcatattcaatttcatcaagccttgtatacttctgcttaaatgtaattcaatccttggttgttatggcaaacagaaacccaacaaaaagacagactctggtactatggtaacagagccactttatcatttggtcaaaatttgacattatgattagctttgtagaactgacctgtttatttggcaatgctgttaaaggatcatttcggtttcagacttcaaagttgattaataaatttaatcttaactttttattcactggaatcaaaacgatggttggtactgtgtttactgtttaaaatgaagtactaaagccctgagaactgcacctcattttctttatccagaaattgtgcttatatattttcctgtcaggtttaaaaagatgttttaattcataaattattgttttcattgacattaaaggactgtgatattgaaaaaaaaaaaaaaaaaa

SEQ ID NO: 98 - protein, 354 aa, Homo sapiens
MAFFCILRKILGKFYSWTPGFGAALPASYVAILIERKSRRGLLTIYMANLVLLFCITAAMYMFFFRCKDGLKGFTFSALRFIVGKEEIPTHSFSPEAAYAKVEQKREQHEEKPRRMNMIGLVRKFVDSICKHGPRHRCCKHYEDNCISYCIKGFIRMFSVGYLIQCCLRIPSAFRHLFTQPSRLLSLFYNKENFQLGAFLGSFVSIYKGTSCFLRWIRNLDDELHAIIAGFLAGTSMMFYKSTTISMYLASKLVETMYFKGIEAGKVPYFPHADTIIYSISTAICFQAAVMEVQTLRPSYWKFLLRLTKGKFAVMNRKVLDVFGTGASKHFQDFIPRLDPRYTTVTPELPTEFS

SEQ ID NO: 99 - DNA, 507 bp, Homo sapiens
gattccgcacctccgagtgctggccgggcgagaggctggcggctgggctctcgcgcccctccctgcagggctcaggctctccccctcctgtcttctccgcgctgttttttgtcatggcggccctcagcaagtccatccctcataactgctatgagatcggccacacttggcacccttcctgccgggtctccttcctgcagatcaccgggggcgccctggaggagtccctgaagatctatgctcctctgtacttgattgcagcaattctccggaaacggaaattagactattatttacacaaactactccctgagatcctacaatccgcttcatttctaactgctaatggggccttgtatatggctttcttttgcattttaaggaagatacttggaaaattctactcatggactcctggctttggtgccgctctgccagcatcttatgtggccattctcattgaaagaaaaagcaggagagggctgctcacaatttatatggccaact

SEQ ID NO: 100 - DNA, 23 bp, Homo sapiens
cagagtaaccccgctctcgtgac

SEQ ID NO: 101 - DNA, 30 bp, Homo sapiens
gtgttcaacataattaactcttcaagattg

SEQ ID NO: 102 - DNA, 17 bp, Homo sapiens
gtaaaacgacggccagt

SEQ ID NO: 103 - DNA, 19 bp, Homo sapiens
ggaaacagctatgaccatg

SEQ ID NO: 104 - DNA, 24 bp, Homo sapiens
ccaaagccaggagtccatgagtag

SEQ ID NO: 105 - DNA, 27 bp, Homo sapiens
tggtctagtcaggaaatttgtggattc

SEQ ID NO: 106 - DNA, 27 bp, Homo sapiens
gaatccacaaatttcctgactagacca

SEQ ID NO: 107 - DNA, 23 bp, Homo sapiens
ctactcatggactcctggctttg

SEQ ID NO: 108 - DNA, 26 bp, Homo sapiens
gcacaacaattcccatgtatttagcg

SEQ ID NO: 109 - DNA, 26 bp, Homo sapiens
cgctaaatacatgggaattgttgtgc

SEQ ID NO: 110 - DNA, 24 bp, Homo sapiens
gacggtggtacactcttgagaaaa

SEQ ID NO: 111 - DNA, 30 bp, Homo sapiens
gaagatgggaaaacattgtataatttaagc

(19) United States
(12) Patent Application Publication
(10) Pub. No.: US 2004/0023276 A1
(43) Pub. Date: Feb. 5, 2004

(54) LXR-ligand induced genes and proteins

(76) Inventors: Teresa R. Ward, Seattle, WA (US); Mao Mao, Redmond, WA (US); Peter S. Linsley, Seattle, WA (US); Erik Lund, Brooklyn, NY (US)

Correspondence Address: R. Douglas Bradley, Rosetta Inpharmatics LLC, - Legal Department, 12040 - 115th Avenue NE, Kirkland, WA, 98034, US

(21) Appl. No.: 10/429,160

(22) Filed: May 2, 2003

Related U.S. Application Data
(60) Provisional application No. 60/377,714, filed on May 3, 2002.

(51) Int. Cl. C12Q 1/68; C07H 21/04; C12P 21/02; C12N 5/06; C07K 14/705

(52) U.S. Cl. 435/6; 435/69.1; 435/320.1; 435/325; 530/350; 536/23.5

(57) ABSTRACT

The present invention features nucleic acids and polypeptides encoding LXR-Ligand Induced I (LXRLI1). Treatment of human cells with acetylpodocarpic dimer, a LXR-agonist compound, results in an increase in LXRLI1 gene expression. The cDNA sequence of LXRLI1 is provided by SEQ ID NO 1. The amino acid sequence for LXRLI1 is provided by SEQ ID NO 2. The present invention also defines an LXR-ligand induced gene and provides methods for using gene expression profiles of this set of LXR regulated genes to measure LXR activity in a subject, to diagnose a disease or disorder involving LXR activity, to screen for compounds that change the activity of LXR and to classify LXR ligands.

### BACKGROUND OF THE INVENTION

The references cited herein are not admitted to be prior art to the claimed invention.

Cholesterol is used for the synthesis of bile acids in the liver, the manufacture and repair of cell membranes, and the synthesis of steroid hormones. There are both exogenous and endogenous sources of cholesterol. The average American consumes about 450 mg of cholesterol each day and produces an additional 500 to 1,000 mg in the liver and other tissues. Another source is the 500 to 1,000 mg of biliary cholesterol that is secreted into the intestine daily; about 50 percent is reabsorbed (enterohepatic circulation). Excess accumulation of cholesterol in the arterial walls can result in atherosclerosis, which is characterized by plaque formation. The plaques inhibit blood flow, promote clot formation and can ultimately cause heart attacks, stroke and claudication. Development of therapeutic agents for the treatment of atherosclerosis and other diseases associated with cholesterol metabolism has been focused on achieving a more complete understanding of the biochemical pathways involved.

Atherosclerosis is the leading cause of death in western industrialized countries. The risk of developing atherosclerosis is directly related to plasma levels of LDL cholesterol and inversely related to HDL cholesterol levels. It is generally accepted that HDL is involved in the transport of cholesterol from extrahepatic tissues to the liver, a process known as reverse cholesterol transport (RCT), as described by Pieters, et al. (1994 Biophys. Acta 1225:125), and mediates the transport of cholesteryl ester to steroidogenic tissues for hormone synthesis, as described by Andersen and Dietschy (1981 J. Biol. Chem. 256:7362). High density lipoprotein (HDL) and low density lipoprotein (LDL) are cholesterol transport particles whose plasma concentrations are directly (LDL) and inversely (HDL) correlated with risk for atherosclerosis.

Recently, liver X receptors (LXRs) and genes transcriptionally regulated by LXRs were identified as key components in cholesterol homeostasis. The LXRs were first identified as orphan members of the nuclear receptor superfamily whose ligands and functions were unknown. Two LXR proteins (α and β) are known to exist in mammals. The expression of LXRα is restricted, with the highest levels being found in the liver, and lower levels found in kidney, intestine, spleen, and adrenals (see Willy, et al., 1995 Genes Dev. 9:1033-45). LXRβ is rather ubiquitous, being found in nearly all tissues examined. Recent studies on the LXRs indicate that they are activated by certain naturally occurring, oxidized derivatives of cholesterol, including 22(R)-hydroxycholesterol, 24(S)-hydroxycholesterol, 24, 25(S)-epoxycholesterol and 27-OH cholesterol (see Spencer et al, 2001 J. Med. Chem. 44:886-97; Lehmann, et al., 1997 J. Biol. Chem. 272:3137-40; Janowski et al., 1996 Nature 383:728-31; and Fu et al., 2001 J. Biol. Chem. 276:38378-87). The expression pattern of LXRs and their oxysterol ligands provided the first hint that these receptors may play a role in cholesterol metabolism (see Janowski, et al., 1996 Nature 383:728-31).

As noted above, cholesterol metabolism in mammals occurs via conversion into steroid hormones or bile acids. The role of LXRs in cholesterol homeostasis was first postulated to involve the pathway of bile acid synthesis, in which cholesterol 7α-hydroxylase (CYP7A) operates in a rate-limiting manner. Support for this proposal was provided when additional experiments found that the CYP7A promoter contained a functional LXR response element that could be activated by LXR/retinoid X receptor (RXR) heterodimers in an oxysterol- and retinoid-dependent manner. Confirmation of LXR function as a transcriptional control point in cholesterol metabolism was made using knockout mice, particularly those lacking the gene encoding oxysterol receptor LXRα (see Peet, et al., 1998 Cell 93:693-704).

Mice lacking the receptor LXRα (e.g., knockout or (−/−) mice) lost their ability to respond normally to increases in dietary cholesterol and were unable to tolerate any cholesterol in excess of that synthesized de novo. LXRα (−/−) mice did not induce transcription of the gene encoding CYP7A when fed diets containing additional cholesterol. This resulted in an accumulation of large amounts of cholesterol and impaired hepatic function in the livers of LXRα (−/−) mice. These results, and others, further established the role of LXRα as an essential regulatory component of cholesterol homeostasis, in particular reverse cholesterol transport. Indeed, it is now well established that LXR regulated gene products play an integral role in both lipid and cholesterol homeostasis (Edwards, et al., 2002 J. Lipid Res. 43:2-12; Sparrow, et al., 2002 J. Biol. Chem. 277: 10021-7; Chawla, et al., 2001 Science 294:1866-70; Schultz, et al., 2000 Genes & Dev. 14:2831-8; and Menke et al., July 2002 Endocrinology, in press).

Because of the key role of cholesterol and lipid metabolism in the development of atherosclerosis and other heart diseases, much effort has been given to identifying ligand molecules that bind to LXRs and thereby modulate the expression of genes that are transcriptionally activated by LXR/RXR (see, for example, U.S. Pat. No. 6,316,503; WO 01/41704; Repa, et al., 2000 Science 289:1524-9; Schultz, et al., 2000 Genes & Dev. 14:2831-8; and Sparrow, et al., 2002 J. Biol. Chem. 277: 10021-7). Such LXR agonist and antagonists compounds are useful as therapeutic agents for the treatment of disorders associated with bile acid and cholesterol metabolism, including cholesterol gallstones, atherosclerosis, lipid storage diseases, obesity, and diabetes. Modulators of LXR activity can also be used to treat disease states associated with serum hypercholesterolemia, such as coronary heart disease. For example, LXR ligands that increase the expression of the ABCA1 gene have utility as drugs to increase the levels of high density lipoproteins (HDL) and thereby decrease the risk of atherosclerosis, myocardial infarction and related conditions such as peripheral vascular disease, ischemic stroke (WO 01/41704).

A number of genes have been shown to be transcriptionally activated by LXR upon exposure of cells to naturally occurring, oxidized derivatives of cholesterol, and by synthetic compounds, such as acetylpodocarpic dimer (APD) (Sparrow, et al., 2002 J. Biol. Chem. 10.1074/jbc.M1108225200) and nonsteroidal LXR ligand compounds T1317 and GW3965 (Joseph, et al., 2002 J. Biol. Chem. 10.1074/jbc.M111041200), all of which activate LXR and induce increased expression of LXR regulated genes. For example, APD has been shown to induce ABCA1, a lipid pump that effluxes cholesterol and phospholipid out of cells (Sparrow, et al., 2002 J. Biol. Chem. 10.1074/jbc.M1108225200). Other genes that have been reported as being regulated by LXRs are: ABCG1, encoding a transmembrane lipid pump protein (Venkateswaran, et al., 2000 J. Biol. Chem. 275:14700-7; Kennedy, et al., 2001 J. Biol. Chem. 276:39438-47); SCD-1, encoding a stearoyl-CoA desaturase involved in fatty acid biosynthesis ( Schultz, et al., 2000 Genes & Development 14:2831-38); apoE, encoding apolipoprotein E (Laffitte, et al., 2001 Proc. Nat. Acad. Sci. 98:507-12); FAS, encoding fatty acid synthase (Joseph, et al., 2002 J. Biol. Chem. /jbc.M111041200); and LPL, encoding lipoprotein lipase (Zhang, et al., 2001, 276:43018-24).

### SUMMARY OF THE INVENTION

Gene expression profiles have been evaluated to identify genes that are coregulated over many reference conditions as a method for gene discovery and functional characterization of ESTs, partial cDNA, and computationally predicted coding sequences whose functions are unknown. In particular, several different cell types were treated with a LXR-ligand to identify new genes that are coregulated with genes known to be transcriptionally regulated by LXR. Using this method transcripts have been identified, cloned and sequenced that bridge sequence regions that were previously identified as representing distinct transcripts. The novel polynucleotides of the present invention encode a novel polypeptide and are transcriptionally induced by the LXR-agonist compound APD.

More specifically, the present invention features polynucleotides encoding LXR-Ligand Induced 1 (LXRLI1) and LXRLI1 polypeptides. LXRLI1 is transcriptionally activated by compounds that are capable of binding to nuclear receptor LXR which in turn activates transcription of target genes. The cDNA sequence encoding LXRLI1 is provided by SEQ ID NO 1. The amino acid sequence for LXRLI1 is provided by SEQ ID NO 2.

Thus, a first aspect of the present invention describes a purified LXRLI1 nucleic acid. The nucleic acid comprises SEQ ID NO 1 or the complement thereof. Reference to the presence of one region does not indicate that another region is not present. For example, in different embodiments the nucleic acid can comprise or consist of a nucleic acid encoding for SEQ ID NO 2 and can comprise or consist of the nucleic acid sequence of SEQ ID NO 1.

Another aspect of the present invention describes a purified LXRLI1 polypeptide. The polypeptide can comprise or consist of the amino acid sequence of SEQ ID NO 2.

Another aspect of the present invention describes an expression vector. The expression vector comprises a nucleotide sequence encoding a polypeptide comprising or consisting of SEQ ID NO 2, wherein the nucleotide sequence is transcriptionally coupled to an exogenous promoter. Alternatively, the nucleotide sequence comprises or consists of SEQ ID NO 1 and is transcriptionally coupled to an exogenous promoter.

Another aspect of the present invention describes a recombinant cell comprising an expression vector comprising or consisting of the above-described sequences and the promoter is recognized by an RNA polymerase present in the cell. Another aspect of the present invention, describes a recombinant cell made by a process comprising the step of introducing into the cell an expression vector comprising a nucleotide sequence comprising or consisting of SEQ ID NO 1, or a nucleotide sequence encoding a polypeptide comprising or consisting of an amino acid sequence of SEQ ID NO 2, wherein the nucleotide sequence is transcriptionally coupled to an exogenous promoter. The expression vector can be used to insert recombinant nucleic acid into the host genome or can exist as an autonomous piece of nucleic acid.

Another aspect of the present invention describes a method of producing a LXRLI1 polypeptide comprising at least nine contiguous amino acids of SEQ ID NO 2. The method involves the step of growing a recombinant cell containing an inventive expression vector under conditions wherein the polypeptide is expressed from the expression vector.

Another aspect of the present invention features a purified antibody preparation comprising an antibody that binds to LXRLI1.

Another aspect of the invention provides a method of estimating LXR activity in a subject, comprising the steps of: A method of estimating LXR activity in a subject, comprising: measuring a transcript level in a sample of mRNA or nucleic acid derived therefrom from the subject, wherein the transcript comprises a nucleotide sequence selected from the group consisting of SEQ ID NO 1, SEQ ID NO 13, SEQ ID NO 15, SEQ ID NO 17, SEQ ID NO 19, SEQ ID NO 21, SEQ ID NO 23, SEQ ID NO 25, SEQ ID NO 27, SEQ ID NO 29, SEQ ID NO 31, SEQ ID NO 33, SEQ ID NO 35, SEQ ID NO 37, SEQ ID NO 39, SEQ ID NO 41, SEQ ID NO 43, SEQ ID NO 45, SEQ ID NO 47,SEQ ID NO 49, SEQ ID NO 51, SEQ ID NO 53, SEQ ID NO 55, SEQ ID NO 57, SEQ ID NO 59, SEQ ID NO 61, SEQ ID NO 63, SEQ ID NO 65, SEQ ID NO 67, SEQ ID NO 69, SEQ ID NO 71, SEQ ID NO 73, SEQ ID NO 74, SEQ ID NO 76, SEQ ID NO 77, SEQ ID NO 79, SEQ ID NO 81, SEQ ID NO 82, SEQ ID NO 84, SEQ ID NO 85, SEQ ID NO 86, SEQ ID NO 87, SEQ ID NO 88, SEQ ID NO 89, SEQ ID NO 90, SEQ ID NO 91, SEQ ID NO 92, SEQ ID NO 93, SEQ ID NO 94 and SEQ ID NO 95; and comparing the measured level of the transcript to the level of the transcript measured in a control sample; wherein the level of transcript measured in the sample from the subject as compared to the level of transcript measured in the control sample provides an estimate of LXR activity in the subject sample.

In another aspect of the invention a method is provided for estimating LXR activity in a subject comprising the steps of: detecting the presence of transcripts in a sample comprising mRNA or nucleic acid derived therefrom from a subject, wherein said transcript encodes a polypeptide comprising an amino acid sequence selected from the group consisting of SEQ ID NO 2, SEQ ID NO 14, SEQ ID NO 16, SEQ ID NO 18, SEQ ID NO 20, SEQ ID NO 22, SEQ ID NO 24, SEQ ID NO 26, SEQ ID NO 28, SEQ ID NO 30, SEQ ID NO 32, SEQ ID NO 34, SEQ ID NO 36, SEQ ID NO 38, SEQ ID NO 40, SEQ ID NO 42, SEQ ID NO 44, SEQ ID NO 46,SEQ ID NO 48,SEQ ID NO 50, SEQ ID NO 52, SEQ ID NO 54, SEQ ID NO 56, SEQ ID NO 58, SEQ ID NO 60, SEQ ID NO 62, SEQ ID NO 64, SEQ ID NO 66,SEQ ID NO 68,SEQ ID NO 70,SEQ ID NO 72,SEQ ID NO 75, SEQ ID NO 78, SEQ ID NO 80, SEQ ID NO 83, and SEQ ID NO 96; and comparing the measured level of the transcript to the level of the transcript measured in a control sample; wherein the level of transcript measured in the sample from the subject as compared to the level of transcript measured in the control sample provides an estimate of LXR activity in the subject sample.

In another embodiment of the invention, the above described methods of measuring LXR activity are used to diagnose a disease or disorder involving LXR activity in a sample by detecting increased or decreased in transcript level relative to the amount present in an analogous sample from a subject not having the disease or disorder, or not subjected to therapy.

Alternatively, the above-described methods of measuring LXR activity are used to screen for a compound that changes the activity of LXR, wherein the compound changes the level of LXR activity in a sample from the subject contacted with the compound relative to the level of LXR activity present in an analogous sample from the subject not contacted with the compound.

Yet another aspect of the present invention provides a method for classifying a test LXR-ligand comprising detecting a difference in the expression of a plurality of genes in a first cell sample contacted by the test LXR-ligand compared to the expression of the plurality of genes a second cell sample contacted by a reference LXR-ligand, the plurality of genes consisting of at least five genes corresponding to markers listed in Tables 1, wherein the test LXR-ligand is classified as a full activity LXR-ligand if each gene in the plurality of genes is similarly regulated in the first and second cell samples, and, the test LXR-ligand is classified as a partial activity LXR-ligand if fewer than all of the genes in the plurality of genes are similarly regulated in the first cell sample as compared to the second cell sample.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

### BRIEF DESCRIPTION OF THE FIGURES

FIG. 1 illustrates co-clustering of expression profiles for genes that were known to be regulated by LXR-ligands with other genes, ESTs and predicted transcripts (collectively “markers”) whose regulation by LXR-ligands was unknown. FlexJet® microarrays representing 25,000 mRNA and EST clusters were hybridized to a mixture of cRNAs from untreated versus treated cells of various types. A total of 297 markers were identified as being differentially regulated with a log ratio of >0.2 with a P-value of <0.01 in at least two of the twelve experiments. These gene expression profiles comprising differentially expressed array markers meeting these criteria were then analyzed using a two dimensional hierarchical clustering algorithm. Markers were grouped by greatest similarity of regulation over all experiments (Y-axis) and the experiments showing the greatest similarities in gene regulation (X-axis). Only a section of the total data set is shown in FIG. 1 (55 genes and 12 experiments). The numbers on the Y-axis correspond to the various cell types and compound treatments the cells received prior to measurement of gene expression, as follows:

1) THP-1 cells, no compound vs. exposure to 0.1 μM Zaragozic Acid for 6 hours;

2) THP-1 cells, no compound vs. exposure to 0.1 μM Zaragozic Acid for 20 hours;

3) THP-1 cells, no compound vs. exposure to 0.1 μM Acetyl-Podocarpic Dimer (APD) for 6 hours;

4) THP-1 cells, no compound vs. exposure to 0.1 μM APD for 20 hours;

5) Macrophage cells, no compound vs. exposure to 0.1 μM APD for 24 hours;

6) Hepatocytes, no compound vs. exposure to 0.1 μM APD for 24 hours;

7) Hepatocytes, no compound vs. exposure to 0.01 μM APD for 24 hours;

8) Hepatocytes, no compound vs. exposure to 0.001 μM APD for 24 hours;

9) Hepatocytes, no compound vs. exposure to 0.1 mM Fenofibrate for 24 hours;

10) Hepatocytes, no compound vs. exposure to 0.03 mM Fenofibrate for 24 hours;

11) Hepatocytes, no compound vs. exposure to 0.01 mM Fenofibrate for 24 hours; and

12) Hepatocytes, no compound vs. exposure to 0.1 μM Glucagon for 24 hours.

On the top X-axis, sequence annotation designations are provided for each marker represented in the LXR-ligand induced geneset. Markers up-regulated in a particular experiment are colored light-gray; markers down-regulated in that experiment are colored black; and markers showing no regulation in a particular experiment are colored medium-gray.

FIG. 2 illustrates the use of gene expression profiles comprising the fifty-five markers identified in FIG. 2 to classify compounds affecting cholesterol metabolism. The numbers on the Y-axis correspond to the various cell types and compound treatments the cells received prior to measurement of gene expression, as follows:

1) THP-1 cells, no compound vs. exposure to 0.1 μM APD for 6 hours;

2) THP-1 cells, no compound vs. exposure to 0.1 μM APD for 20 hours;

3) Macrophage cells, no compound vs. exposure to 0.1 μM APD for 24 hours;

4) Hepatocytes, no compound vs. exposure to 0.001 μM APD for 24 hours;

5) Hepatocytes, no compound vs. exposure to 0.01 μM APD for 24 hours;

6) Hepatocytes, no compound vs. exposure to 0.1 μM APD for 24 hours;

7) THP-1 cells, no compound vs. exposure to 10 μM 22(R)-hydroxycholesterol (22RHC) for 3 hours;

8) THP-1 cells, no compound vs. exposure to 10 μM 22RHC for 6 hours;

9) THP-1 cells, no compound vs. exposure to 10 μM 22RHC for 20 hours;

10) THP-1 cells, no compound vs. exposure to 200 μg protein/ml Acetylated Low Density Lipoprotein (AcLDL) for 3 hours;

11) THP-1 cells, no compound vs. exposure to 200 μg protein/ml AcLDL for 6 hours;

12) THP-1 cells, no compound vs. exposure to 200 μg protein/ml AcLDL for 20 hours;

13) THP-1 cells, no compound vs. exposure to 0.5 mM CD and Cholesterol at 24 μg/ml for 3 hours;

14) THP-1 cells, no compound vs. exposure to 0.5 mM CD and Cholesterol at 24 μg/ml for 6 hours;

15) THP-1 cells, no compound vs. exposure to 0.5 mM CD and Cholesterol at 24 μg/ml for 20 hours;

16) THP-1 cells, no compound vs. exposure to 0.1 μM Zaragozic Acid for 6 hours;

17) THP-1 cells, no compound vs. exposure to 0.1 μM Zaragozic Acid for 20 hours;

18) THP-1 cells, no compound vs. exposure to 2.0 mM Methyl-β-Cyclodextrin (CD) for 1 hour followed by recovery for 3 hours;

19) THP-1 cells, no compound vs. exposure to 2.0 mM CD for 1 hour followed by recovery for 3 hours; and

20) THP-1 cells, no compound vs. exposure to 2.0 mM CD for 1 hour followed by recovery for 3 hours.

On the top X-axis, sequence annotation designations are provided for each marker represented in the LXR-ligand classification geneset. Markers up-regulated in a particular experiment are colored light-gray; markers down-regulated in that experiment are colored black; and markers showing no regulation in a particular experiment are colored medium-gray.

### DEFINITIONS

Unless defined otherwise, all technical and scientific terms used herein have the meaning commonly understood by one of ordinary skill in the art to which this invention belongs.

As used herein, underscoring or italicizing the name of a gene shall indicate the gene, in contrast to its encoded protein product which is indicated by the name of the gene in the absence of any underscoring or italicizing. For example, “LXRLI1” shall mean polynucleotides encoding the protein product “LXRLI1.”

As used herein, “LXR” includes all subtypes of this receptor and corresponding polynucleotides that encode such subtypes. Specifically LXR includes LXRα and LXRβ, and a ligand of LXR should be understood to include a ligand of LXRα or LXRβ.

As used herein, “ligand” includes an agonist, partial agonist or antagonist of LXR. The ligand may be selective for LXRα or LXRβ. Reference to a compound as an agonist or antagonist does not mean that that compound does not have a different biochemical effect on the target receptor under a different circumstance, e.g., in a different cell type, tissue type or disease state.

As used herein, an “isolated nucleic acid” is a nucleic acid molecule that exists in a physical form that is nonidentical to any nucleic acid molecule of identical sequence as found in nature; “isolated” does not require, although it does not prohibit, that the nucleic acid so described has itself been physically removed from its native environment. For example, a nucleic acid can be said to be “isolated” when it includes nucleotides and/or internucleoside bonds not found in nature. When instead composed of natural nucleosides in phosphodiester linkage, a nucleic acid can be said to be “isolated” when it exists at a purity not found in nature, where purity can be adjudged with respect to the presence of nucleic acids of other sequence, with respect to the presence of proteins, with respect to the presence of lipids, or with respect the presence of any other component of a biological cell, or when the nucleic acid lacks sequence that flanks an otherwise identical sequence in an organism's genome, or when the nucleic acid possesses sequence not identically present in nature. As so defined, “isolated nucleic acid” includes nucleic acids integrated into a host cell chromosome at a heterologous site, recombinant fusions of a native fragment to a heterologous sequence, recombinant vectors present as episomes or as integrated into a host cell chromosome.

A “purified nucleic acid” represents at least 10% of the total nucleic acid present in a sample or preparation. In preferred embodiments, the purified nucleic acid represents at least about 50%, at least about 75%, or at least about 95% of the total nucleic acid in a isolated nucleic acid sample or preparation. Reference to “purified nucleic acid” does not require that the nucleic acid has undergone any purification and may include, for example, chemically synthesized nucleic acid that has not been purified.

The phrases “isolated protein”, “isolated polypeptide”, “isolated peptide” and “isolated oligopeptide” refer to a protein (or respectively to a polypeptide, peptide, or oligopeptide) that is nonidentical to any protein molecule of identical amino acid sequence as found in nature; “isolated” does not require, although it does not prohibit, that the protein so described has itself been physically removed from its native environment. For example, a protein can be said to be “isolated” when it includes amino acid analogues or derivatives not found in nature, or includes linkages other than standard peptide bonds. When instead composed entirely of natural amino acids linked by peptide bonds, a protein can be said to be “isolated” when it exists at a purity not found in nature—where purity can be adjudged with respect to the presence of proteins of other sequence, with respect to the presence of non-protein compounds, such as nucleic acids, lipids, or other components of a biological cell, or when it exists in a composition not found in nature, such as in a host cell that does not naturally express that protein.

As used herein, a “purified polypeptide” (equally, a purified protein, peptide, or oligopeptide) represents at least 10% of the total protein present in a sample or preparation, as measured on a weight basis with respect to total protein in a composition. In preferred embodiments, the purified polypeptide represents at least about 50%, at least about 75%, or at least about 95% of the total protein in a sample or preparation. A “substantially purified protein” (equally, a substantially purified polypeptide, peptide, or oligopeptide) is an isolated protein, as above described, present at a concentration of at least 70%, as measured on a weight basis with respect to total protein in a composition. Reference to “purified polypeptide” does not require that the polypeptide has undergone any purification and may include, for example, chemically synthesized polypeptide that has not been purified.

As used herein, the term “microarray” and the equivalent phrase “nucleic acid microarray” refer to a substrate-bound collection of a plurality of nucleic acids, hybridization to each of the plurality of bound nucleic acids being separately detectable. The substrate can be solid or porous, planar or non-planar, unitary or distributed. As so defined, the term “microarray” and the phrase “nucleic acid microarray” include, but are not limited to, all the devices so called in Schena (ed.), DNA Microarrays: A Practical Approach (Practical Approach Series), Oxford University Press (1999) (ISBN: 0199637768); 1999 Nature Genet. 21(suppl):1-60; Schena (ed.), Microarray Biochip: Tools and Technology, Eaton Publishing Company/BioTechniques Books Division (2000) (ISBN: 1881299376); and Hughes, et al., 2001 Nature Biotechnol. 19:342-7. As so defined, the term “microarray” and the phrase “nucleic acid microarray” also include substrate-bound collections of plural nucleic acids in which the plurality of nucleic acids are distributably disposed on a plurality of beads, rather than on a unitary planar substrate, as is described, inter alia, in Brenner, et al., 2000 Proc. Natl. Acad. Sci. 97:16650-7); in such case, the term “microarray” and the phrase “nucleic acid microarray” refer to the plurality of beads in aggregate.

As used herein, the term “marker” in the context of microarrays and gene expression profiles derived therefrom, refers to a gene, EST, predicted transcript or a polypeptide encoded by any of the preceding polynucleotides that is represented on a mircroarray by a hybridization probe that is complementary and hybridizable to any portion of a corresponding mRNA transcript.

As used herein, the term “geneset” refers to a plurality of genes, ESTs and predicted transcripts that are coregulated under two or more biological conditions.

As used herein, the phrase “similarly regulated” in the context of gene expression means that a measured level of gene expression of the same marker in two or more test samples indicates that the marker is up-regulated or down-regulated to a statistically significant threshold as compared to a control sample. The control sample can be any type of cell or tissue sample that serves as an appropriate baseline for determining differential gene expression. For example, the control sample can be from normal, i.e., non-diseased cells or tissue; from cells or tissue not exposed to a test compound; from cells or tissues of a different kind as compared to the test samples, e.g., a different cell type or tissues type, or a different developmental; or a pool of a plurality of samples from any of above. The statistical significance of a gene expression result can be determined using any one of a variety of different standard statistical tests that are well known in the art (see for example, U.S. Pat. No. 6,203,987 and U.S. Pat. No. 6,351,712).

As used herein, the term “antibody” refers to a polypeptide, at least a portion of which is encoded by at least one immunoglobulin gene, or fragment thereof, and that can bind specifically to a desired target molecule. The term includes naturally-occurring forms, as well as fragments and derivatives. Fragments within the scope of the term “antibody” include those produced by digestion with various proteases, those produced by chemical cleavage and/or chemical dissociation, and those produced recombinantly, so long as the fragment remains capable of specific binding to a target molecule. Among such fragments are Fab, Fab′, Fv, F(ab)′₂, and single chain Fv (scFv) fragments. Derivatives within the scope of the term include antibodies (or fragments thereof) that have been modified in sequence, but remain capable of specific binding to a target molecule, including: interspecies chimeric and humanized antibodies; antibody fusions; heteromeric antibody complexes and antibody fusions, such as diabodies (bispecific antibodies), single-chain diabodies, and intrabodies (see, e.g., Marasco (ed.), Intracellular Antibodies: Research and Disease Applications, Springer-Verlag New York, Inc. (1998) (ISBN: 3540641513). As used herein, antibodies can be produced by any known technique, including harvest from cell culture of native B lymphocytes, harvest from culture of hybridomas, recombinant expression systems, and phage display.

As used herein, a “purified antibody preparation” is a preparation where at least 10% of the antibodies present bind to the target ligand. In preferred embodiments, antibodies binding to the target ligand represent at least about 50%, at least about 75%, or at least about 95% of the total antibodies present. Reference to “purified antibody preparation” does not require that the antibodies in the preparation have undergone any purification.

As used herein, “specific binding” refers to the ability of two molecular species concurrently present in a heterogeneous (inhomogeneous) sample to bind to one another in preference to binding to other molecular species in the sample. Typically, a specific binding interaction will discriminate over adventitious binding interactions in the reaction by at least two-fold, more typically by at least 10-fold, often at least 100-fold; when used to detect analyte, specific binding is sufficiently discriminatory when determinative of the presence of the analyte in a heterogeneous (inhomogeneous) sample. Typically, the affinity or avidity of a specific binding reaction is least about 10⁻⁷ M, with specific binding reactions of greater specificity typically having affinity or avidity of at least 10⁻⁸ M to at least about 10⁻⁹ M.

The term “antisense”, as used herein, refers to a nucleic acid molecule sufficiently complementary in sequence, and sufficiently long in that complementary sequence, as to hybridize under intracellular conditions to (i) a target mRNA transcript or (ii) the genomic DNA strand complementary to that transcribed to produce the target mRNA transcript.

The term “reverse cholesterol transport” describes the transport of cholesterol from extrahepatic tissues to the liver where it maybe catabolized and eliminated.

The term “subject”, as used herein refers to an organism and to cells or tissues derived therefrom. For example the organism may be an animal, including but not limited to animals such as cows, pigs, horses, chickens, cats, dogs, etc., and is usually a mammal, and most commonly human.

### DETAILED DESCRIPTION OF THE INVENTION

The present invention relates to the amino acid sequence of human LXRLI1 and to nucleotide sequences encoding this protein. SEQ ID NO 1) is a cDNA sequence containing a full open reading frame that encodes LXRLI1 (SEQ ID NO 2). Transcription of the LXRLI1 gene was found to be induced by the LXRα/β agonist compound acetylpodocarpic dimer (APD).

LXRLI1 polynucleotides encoding LXRLI1 and LXRLI1 proteins, as exemplified and enabled herein include a number of specific, substantial and credible utilities. For example, LXRLI1 encoding nucleic acids were cloned from a human source (see Example 2). Such nucleic acids can be used as hybridization probes to distinguish between cells that produce LXRLI1 transcripts from human or non-human cells (including bacteria) that do not produce such transcripts. Similarly, antibodies specific for LXRLI1 can be used to distinguish between cells that express LXRLI1 from human or non-human cells (including bacteria) that do not express LXRLI1.

Based on LXRLI1 being a LXR regulated gene whose gene product protein plays a role in cholesterol/lipid metabolism, LXRLI1 provides a target to achieve a beneficial effect in a subject. Preferably, LXRLI1 activity is modulated to achieve one or more of the following: prevent or reduce the risk of occurrence, or recurrence where the potential exist, of cholesterol gallstones, atherosclerosis, lipid storage diseases, obesity, diabetes, Alzheimer's disease, or hypercholesterolemia. Compounds that treat hypercholesterolemia are particularly important because of the cause-and-effect relationship between hypercholesterolemia and morbidity and mortality from coronary artery disease (CAD) (for a review see, Witztum, In, Goodman & Gilman's The Pharmacological Basis of Therapeutics, 9th Ed., McGraw-Hill, New York, 1996, Ch. 36, pp. 875-897).

Modulation of LXRLI1 activity is preferably achieved by evoking a response at the LXR nuclear receptor protein or by altering a response evoked by a LXR nuclear receptor agonist or antagonist. Compounds modulating LXR receptor activity include agonists, antagonists, and allosteric modulators. Generally, LXR-agonists and allosteric modulators positively affecting LXRLI1 activity will be used to increase reverse cholesterol transport. It is believed that plasma HDL particles play a major role in the reverse transport process, acting as scavengers of tissue cholesterol. The evidence linking elevated serum cholesterol to coronary heart disease is overwhelming. For example, atherosclerosis is a slowly progressive disease characterized by the accumulation of cholesterol within the arterial wall. Compelling evidence supports the concept that lipids deposited in atherosclerotic lesions are derived primarily from plasma LDL; thus, LDLs have popularly become known as the “bad” cholesterol. In contrast, HDL serum levels correlate inversely with coronary heart disease—indeed, high serum levels of HDL are regarded as a negative risk factor. It is hypothesized that high levels of plasma HDL are not only protective against coronary artery disease, but may actually induce regression of atherosclerotic plaques (e.g., see Badimon, et al., 1992 Circulation 86 (Suppl. III):86-94). Thus, HDL have popularly become known as the “good” cholesterol and treatments that increase the level of RCT have a beneficial therapeutic effect on a subject.

LXRLI1 activity can also be affected by modulating LXRLI1 expression. Compounds modulating LXRLI1 expression include a cloned polynucleotide encoding LXRLI1 that can express LXRLI1 in vivo, antisense nucleic acids targeted to LXRLI1 transcripts and enzymatic nucleic acids, such as ribozymes and RNAi, targeted to LXRLI1 transcripts.

Preferably, LXRLI1 activity is modulated to achieve a therapeutic effect upon diseases in which cholesterol metabolism is in need of adjustment in a subject. For example, atherosclerosis can be treated by modulating LXRLI1 activity to achieve, for instance, increased levels of RCT and HDL. In other embodiments, the risk of developing atherosclerosis is reduced by modulating LXRLI1 activity to achieve, for example, increased levels of RCT and HDL.

### LXRLI1 NUCLEIC ACID

LXRLI1 nucleic acid contain a region that encodes for a polypeptide comprising or consisting of SEQ ID NO 2 or contains comprises or consists of SEQ ID NO 1. LXRLI1 nucleic acid have a variety of uses, such as being used as a hybridization probe or PCR primer to identify the presence of LXRLI1 nucleic acid; being used as a hybridization probe or PCR primer to identify nucleic acid encoding for proteins related to LXRLI1; and/or being used for recombinant expression of LXRLI1 polypeptides.

Regions in LXRLI1 nucleic acid that do not encode for LXRLI1 amino acids or are not found in SEQ ID NO 1, if present, are preferably chosen to achieve a particular purpose. Examples of additional regions that can be used to achieve a particular purpose include capture regions that can be used as part of a sandwich assay, reporter regions that can be probed to indicate the presence of the nucleic acid, expression vector regions, and regions encoding for other polypeptides.

LXRLI1 nucleic acid also includes transcripts that are transcriptionally regulated by LXR and encode a polypeptide that has a sequence similarity of at least about 85%, preferably at least 95% with SEQ ID NO 2; and nucleic acid having a sequence similarity of at least about 85%, preferably 90% with SEQ ID NO 1. Sequence similarity for nucleic acid can be determined by FASTA. (Pearson 1990, Methods in Enzymology 183, 63-98). In one embodiment, sequence similarity is determined using FASTA search program with the following parameters: MATRIX: BLOSUM50, GAP PENALTIES: open=−12; residue=−2.

The guidance provided in the present application can be used to obtain the nucleic acid sequence encoding for LXRLI1 related proteins from different sources. Obtaining nucleic acids encoding for LXRLI1 related proteins from different sources is facilitated using sets of degenerative probes and primers and by the proper selection of hybridization conditions. Sets of degenerative probes and primers are produced taking into account the degeneracy of the genetic code. Adjusting hybridization conditions is useful for controlling probe or primer specificity to allow for hybridization to nucleic acids having similar sequences.

Techniques employed for hybridization detection and PCR cloning are well known in the art. Nucleic acid detection techniques are described, for example, in Sambrook, et al., in Molecular Cloning, A Laboratory Manual, 2^{nd} Edition, Cold Spring Harbor Laboratory Press, 1989. PCR cloning techniques are described, for example, in White, Methods in Molecular Cloning, volume 67, Humana Press, 1997.

LXRLI1 probes and primers can be used to screen nucleic acid libraries containing, for example, genomic DNA or cDNA. Such libraries are commercially available; and can be produced using techniques such as those described in Ausubel, Current Protocols in Molecular Biology, John Wiley, 1987-1998.

Starting with a particular amino acid sequence and the known degeneracy of the genetic code, a large number of different encoding nucleic acid sequences can be obtained. The degeneracy of the genetic code arises because almost all amino acids are encoded for by different combinations of nucleotide triplets or “codons”. The translation of a particular codon into a particular amino acid is well known in the art (see, e.g., Lewin GENES IV, p. 119, Oxford University Press, 1990). Amino acids are encoded for by codons as follows:

A=Ala=Alanine: codons GCA, GCC, GCG, GCU

C=Cys=Cysteine: codons UGC, UGU

D=Asp=Aspartic acid: codons GAC, GAU

E=Glu=Glutamic acid: codons GAA, GAG

F=Phe=Phenylalanine: codons UUC, UUU

G=Gly=Glycine: codons GGA, GGC, GGG, GGU

H=His=Histidine: codons CAC, CAU

I=Ile=Isoleucine: codons AUA, AUC, AUU

K=Lys=Lysine: codons AAA, AAG

L=Leu=Leucine: codons UUA, UUG, CUA, CUC, CUG, CUU

M=Met=Methionine: codon AUG

N=Asn=Asparagine: codons AAC, AAU

P=Pro=Proline: codons CCA, CCC, CCG, CCU

Q=Gln=Glutarmine: codons CAA, CAG

R=Arg=Arginine: codons AGA, AGG, CGA, CGC, CGG, CGU

S=Ser=Serine: codons AGC, AGU, UCA, UCC, UCG, UCU

T=Thr=Threonine: codons ACA, ACC, ACG, ACU

V=Val=Valine: codons GUA, GUC, GUG, GUU

W=Trp=Tryptophan: codon UGG

Y=Tyr=Tyrosine: codons UAC, UAU

Nucleic acid having a desired sequence can be synthesized using chemical and biochemical techniques. Examples of chemical techniques are described in Ausubel, Current Protocols in Molecular Biology, John Wiley, 1987-1998, and Sambrook et al., in Molecular Cloning, A Laboratory Manual, 2^{nd} Edition, Cold Spring Harbor Laboratory Press, 1989.

Biochemical synthesis techniques involve the use of a nucleic acid template and appropriate enzymes such as DNA and/or RNA polymerases. Examples of such techniques include in vitro amplification techniques such as PCR and transcription based amplification, and in vivo nucleic acid replication. Examples of suitable techniques are provided by Ausubel, Current Protocols in Molecular Biology, John Wiley, 1987-1998, Sambrook et al., in Molecular Cloning, A Laboratory Manual, 2^{nd} Edition, Cold Spring Harbor Laboratory Press, 1989, and U.S. Pat. No. 5,480,784.

LXRLI1 Probes

Probes for LXRLI1 contain a region that can specifically hybridize to LXRLI1 target nucleic acid under appropriate hybridization conditions and can distinguish LXRLI1 nucleic acid from non-target nucleic acids. Probes for LXRLI1 can also contain nucleic acids that are not complementary to LXRLI1 nucleic acid.

Preferably, non-complementary nucleic acid that is present has a particular purpose such as being a reporter sequence or being a capture sequence. However, additional nucleic acid need not have a particular purpose as long as the additional nucleic acid does not prevent the LXRLI1 nucleic acid from distinguishing between target and non-target.

Hybridization occurs through complementary nucleotide bases. Hybridization conditions determine whether two molecules, or regions, have sufficiently strong interactions with each other to form a stable hybrid.

The degree of interaction between two molecules that hybridize together is reflected by the Tm of the produced hybrid. The higher the Tm the stronger the interactions and the more stable the hybrid. Tm is effected by different factors well known in the art such as the degree of complementarity, the type of complementary bases present (e.g., A-T hybridization versus G-C hybridization), the presence of modified nucleic acid, and solution components (e.g., Sambrook, et al., in Molecular Cloning, A Laboratory Manual, 2^{nd} Edition, Cold Spring Harbor Laboratory Press, 1989).

Stable hybrids are formed when the Tm of a hybrid is greater than the temperature employed under a particular set of hybridization assay conditions. The degree of specificity of a probe can be varied by adjusting the hybridization stringency conditions. Detecting probe hybridization is facilitated through the use of a detectable label. Examples of detectable labels include luminescent, enzymatic, and radioactive labels.

Examples of stringency conditions are provided in Sambrook, et al., in Molecular Cloning, A Laboratory Manual, 2^{nd} Edition, Cold Spring Harbor Laboratory Press, 1989. An example of high stringency conditions is as follows: Prehybridization of filters containing DNA is carried out for 2 hours to overnight at 65° C. in buffer composed of 6×SSC, 5× Denhardt's solution, and 100 μg/ml denatured salmon sperm DNA. Filters are hybridized for 12 to 48 hours at 65° C. in prehybridization mixture containing 100 μg/ml denatured salmon sperm DNA and 5-20×10⁶ cpm of ³²P-labeled probe. Washing of filters is done at 37° C. for 1 hour in a solution containing 2×SSC, 0.1% SDS. This is followed by a wash in 0.1×SSC, 0.1% SDS at 50° C. for 45 minutes before autoradiography. Other procedures using conditions of high stringency would include, for example, either a hybridization step carried out in 5×SSC, 5× Denhardt's solution, 50% formamide at 42° C. for 12 to 48 hours or a washing step carried out in.0.2×SSPE, 0.2% SDS at 65° C. for 30 to 60 minutes.

Recombinant Expression

LXRLI1 polypeptides can be expressed from recombinant nucleic acid in a suitable host or in a test tube using a translation system. Recombinantly expressed LXRLI1 polypeptides are preferably used in assays to screen for compounds that bind to LXRLI1 and modulate the activity of the protein.

Preferably, expression is achieved in a host cell using an expression vector. An expression vector contains recombinant nucleic acid encoding for a polypeptide along with regulatory elements for proper transcription and processing. The regulatory elements that may be present include those naturally associated with the recombinant nucleic acid and exogenous regulatory elements not naturally associated with the recombinant nucleic acid. Exogenous regulatory elements such as an exogenous promoter can be useful for expressing recombinant nucleic acid in a particular host.

Generally, the regulatory elements that are present in an expression vector include a transcriptional promoter, a ribosome binding site, a terminator, and an optionally present operator. Another preferred element is a polyadenylation signal providing for processing in eukaryotic cells. Preferably, an expression vector also contains an origin of replication for autonomous replication in a host cell, a selectable marker, a limited number of useful restriction enzyme sites, and a potential for high copy number. Examples of expression vectors are cloning vectors, modified cloning vectors, specifically designed plasmids and viruses.

Expression vectors providing suitable levels of polypeptide expression in different hosts are well known in the art. Mammalian expression vectors well known in the art include, but are not restricted to, pcDNA3 (Invitrogen, Carlsbad Calif.), pSecTag2 (Invitrogen), pMClneo (Stratagene, La Jolla Calif.), pXT1 (Stratagene), pSG5 (Stratagene), pCMVLacl (Stratagene), pCI-neo (Promega), EBO-pSV2-neo (ATCC 37593), pBPV-1(8-2) (ATCC 37110), pdBPV-MMTneo(342-12) (ATCC 37224), pRSVgpt (ATCC 37199), pRSVneo (ATCC 37198), pSV2-dhfr (ATCC 37146) and pUCTag (ATCC 37460), and. Bacterial expression vectors well known in the art include pET11a (Novagen), pBluescript SK (Stratagene, La Jolla), pQE-9 (Qiagen Inc., Valencia), lambda gt11 (Invitrogen), pcDNAII (Invitrogen), and pKK223-3 (Pharmacia). Fungal cell expression vectors well known in the art include pPICZ (Invitrogen) and pYES2 (Invitrogen), Pichia expression vector (Invitrogen). Insect cell expression vectors well known in the art include Blue Bac III (Invitrogen), pBacPAK8 (CLONTECH, Inc., Palo Alto) and PfastBacHT (Invitrogen, Carlsbad).

Recombinant host cells may be prokaryotic or eukaryotic. Examples of recombinant host cells include the following: bacteria such as E. coli; fungal cells such as yeast; mammalian cells such as human, bovine, porcine, monkey and rodent; and insect cells such as Drosophila and silkworm derived cell lines. Commercially available mammalian cell lines include L cells L-M(TK⁻) (ATCC CCL 1.3), L cells L-M (ATCC CCL 1.2), 293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26) and MRC-5 (ATCC CCL 171).

To enhance expression in a particular host it may be useful to modify the sequence provided in SEQ ID NO 1 to take into account codon usage of the host. Codon usage of different organisms are well known in the art (see, Ausubel, Current Protocols in Molecular Biology, John Wiley, 1987-1998, Supplement 33 Appendix 1C).

Expression vectors may be introduced into host cells using standard techniques. Examples of such techniques include transformation, transfection, lipofection, protoplast fusion, and electroporation.

Nucleic acid encoding for a polypeptide can be expressed in a cell without the use of an expression vector employing, for example, synthetic mRNA or native mRNA. Additionally, mRNA can be translated in various cell-free systems such as wheat germ extracts and reticulocyte extracts, as well as in cell based systems, such as frog oocytes. Introduction of mRNA into cell based systems can be achieved, for example, by microinjection.

### LXRLI1 POLYPEPTIDES

LXRLI1 polypeptides contain an amino acid sequence comprising or consisting of SEQ ID NO 2. LXRLI1 polypeptides have a variety of uses, such as providing a marker for LXR activity; being used as an immunogen to produce antibodies binding to LXRLI1; being used as a target to identify compounds binding to the LXRLI1; and/or being used as a target to identify compounds that alter reverse cholesterol transport.

In chimeric polypeptides containing one or more regions from LXRLI1 and one or more regions not from LXRLI1, the region(s) not from LXRLI1 can be used, for example, to achieve a particular purpose or to produce a polypeptide that can substitute for LXRLI1 or a fragment thereof. Particular purposes that can be achieved using chimeric LXRLI1 polypeptides include providing a marker for LXR activity, enhancing an immune response, and altering reverse cholesterol transport.

LXRLI1 polypeptides also include functional LXRLI1 that are induced by LXR activation and have a sequence similarity of at least about 85%, preferably at least 95% with SEQ ID NO 2 Sequence similarity for polypeptides can be deternined by BLAST (Altschul, et al., 1997 Nucleic Acids Res. 25:3389-402). In one embodiment sequence similarity is determined using BLAST search program with the following parameters:

MATRIX:BLOSUM62, PER RESIDUE GAP COST: 11, and Lambda ratio: 1.

Polypeptides can be produced using standard techniques including those involving chemical synthesis and those involving biochemical synthesis. Techniques for chemical synthesis of polypeptides are well known in the art (see e.g., Vincent, in Peptide and Protein Drug Delivery, New York, N.Y., Dekker, 1990).

Biochemical synthesis techniques for polypeptides are also well known in the art. Such techniques employ a nucleic acid template for polypeptide synthesis. The genetic code providing the sequences of nucleic acid triplets coding for particular amino acids is well known in the art (see, e.g., Lewin GENES IV, p. 119, Oxford University Press, 1990). Examples of techniques for introducing nucleic acid into a cell and expressing the nucleic acid to produce protein are provided in references such as Ausubel, Current Protocols in Molecular Biology, John Wiley, 1987-1998, and Sambrook, et al., in Molecular Cloning, A Laboratory Manual, 2^{nd} Edition, Cold Spring Harbor Laboratory Press, 1989.

Functional LXRLI1

Functional LXRLI1 is a membrane bound protein whose expression is altered by LXR and plays a role in cholesterol metabolism, more specifically, reverse cholesterol transport. The identification of the amino acid and nucleic acid sequences of LXRLI1 provide tools for obtaining functional proteins related to LXRLI1 from other sources, for producing LXRLI1 chimeric proteins, and for producing functional derivatives of SEQ ID NO 2.

LXRLI1 polypeptides can be readily identified and obtained based on their sequence similarity to LXRLI1. Both the amino acid and nucleic acid sequences of LXRLI1 can be used to help identify and obtain LXRLI1 polypeptides. For example, SEQ ID NO 2 can be used to produce degenerative nucleic acid probes or primers for identifying and cloning nucleic acid encoding for a LXRLI1 polypeptide, and SEQ ID NO 1 or fragments thereof, can be used under conditions of moderate stringency to identify and clone nucleic acid encoding LXRLI1 polypeptides from a variety of different organisms.

The use of degenerative probes and moderate stringency conditions for cloning is well known in the art. Examples of such techniques are described by Ausubel, Current Protocols in Molecular Biology, John Wiley, 1987-1998, and Sambrook, et al., in Molecular Cloning, A Laboratory Manual, 2^{nd} Edition, Cold Spring Harbor Laboratory Press, 1989.

Starting with LXRLI1 obtained from a particular source, derivatives can be produced that are involved in reverse cholesterol transport. Such derivatives include polypeptides with amino acid substitutions, additions and deletions. Changes to LXRLI1 to produce a derivative having essentially the same properties should be made in a manner not altering the tertiary structure.

Differences in naturally occurring amino acids are due to different R groups. An R group effects different properties of the amino acid such as physical size, charge, and hydrophobicity. Amino acids are can be divided into different groups as follows: neutral and hydrophobic (alanine, valine, leucine, isoleucine, proline, tryptophan, phenylalanine, and methionine); neutral and polar (glycine, serine, threonine, tryosine, cysteine, asparagine, and glutamine); basic (lysine, arginine, and histidine); and acidic (aspartic acid and glutamic acid).

Generally, in substituting different amino acids it is preferable to exchange amino acids having similar properties. Substituting different amino acids within a particular group, such as substituting valine for leucine, arginine for lysine, and asparagine for glutamine are good candidates for not causing a change in polypeptide functioning.

Changes outside of different amino acid groups can also be made. Preferably, such changes are made taking into account the position of the amino acid to be substituted in the polypeptide. For example, arginine can substitute more freely for nonpolar amino acids in the interior of a polypeptide then glutamate because of its long aliphatic side chain (See, Ausubel, Current Protocols in Molecular Biology, John Wiley, 1987-1998, Supplement 33 Appendix 1C).

LXRLI1 Antibodies

Antibodies recognizing LXRLI1 can be produced using a polypeptide containing SEQ ID NO 2 or a fragment thereof as an immunogen. Preferably, a polypeptide used as an immunogen consists of a polypeptide of SEQ ID NO 2 or a SEQ ID NO 2 fragment at least 9 amino acids in length.

Antibodies to LXRLI1 have different uses such as being used to identify the presence of LXRLI1 and to isolate LXRLI1 polypeptides. Identifying the presence of LXRLI1 can be used, for example, to identify cells producing LXRLI1. Such identification provides an additional source of LXRLI1 and can be used to distinguish cells known to produce LXRLI1 from cells that do not produce LXRLI1. For example, antibodies to LXRLI1 can distinguish human cells expressing LXRLI1 from human cells not expressing LXRLI1 or non-human cells (including bacteria) that do not express LXRLI1. Such LXRLI1 antibodies can also be used to determine the effectiveness of LXR ligands, using techniques well known in the art, to detect and quantify changes in the protein levels of LXRLI1 in cellular extracts, and in situ immunostaining of cells and tissues.

Techniques for producing and using antibodies are well known in the art. Examples of such techniques are described in Ausubel, Current Protocols in Molecular Biology, John Wiley, 1987-1998; Harlow, et al., Antibodies, A Laboratory Manual, Cold Spring Harbor Laboratory, 1988; and Kohler, et al., 1975 Nature 256:495-7.

LXRLI1 Binding Assay

LXRLI1 or a fragment thereof can be used in binding studies to identify compounds binding to the protein. Such studies can be performed using different formats including competitive and non-competitive formats. Further competition studies can be carried out using additional compounds determined to bind to LXRLI1.

The particular LXRLI1 sequence involved in ligand binding can be readily identified by using labeled compounds that bind to the protein and different protein fragments. Different strategies can be employed to select fragments to be tested to narrow down the binding region. Examples of such strategies include testing consecutive fragments about 15 amino acids in length starting at the N-terminus, and testing longer length fragments. If longer length fragments are tested, a fragment binding to a compound can be subdivided to further locate the binding region. Fragments used for binding studies can be generated using recombinant nucleic acid techniques.

Preferably, binding studies are performed using LXRLI1 expressed from a recombinant nucleic acid. More preferably, recombinantly expressed LXRLI1 consists of the SEQ ID NO 2 amino acid sequence.

Binding assays can be performed using individual compounds or preparations containing different numbers of compounds. A preparation containing different numbers of compounds having the ability to bind to LXRLI1 can be divided into smaller groups of compounds that can be tested to identify the compound(s) binding to LXRLI1.

Binding assays can be performed using recombinantly produced LXRLI1 present in different environments. Such environments include, for example, cell extracts and purified cell extracts containing the LXRLI1 recombinant nucleic acid; and also include, for example, the use of a purified LXRLI1 polypeptide produced by recombinant means which is introduced into a different environment.

Functional Assays

The identification of LXRLI1 as a LXR regulated gene provides a means for screening for compounds that bind to LXRLI1 protein thereby altering reverse cholesterol transport. Assays involving a functional LXRLI1 polypeptide can be employed for different purposes such as selecting for compounds active at LXRLI1, evaluating the ability of a compound to affect reverse cholesterol transport, and mapping the activity of different LXRLI1 regions. LXRLI1 activity can be measured using different techniques such as detecting a change in the intracellular conformation of LXRLI1, detecting a change in the intracellular location of LXRLI1, or measuring the level of reverse cholesterol transport activity.

Recombinantly expressed LXRLI1 can be used to facilitate determining whether a compound is active at LXRLI1. For example, LXRLI1 can be expressed by an expression vector in a cell line and used in a co-culture growth assay, such as described in WO 99/59037, to identify compounds that bind to LXARAI1.

Techniques for measuring reverse cholesterol transport (RCT), are known in the art. In particular, Sparrow, et al. (2002 J. Biol. Chem. 277: 10021-7) report a method for performing a cholesterol efflux assay on cultured mammalian tissue culture cells using gas chromatography-mass spectrometry. Other RCT assays include, but are not limited to, measurement of efflux of radioactive cholesterol to: 1) exogenous apoA-I or HDL as described in Francis, et al. (1995 J. Clin. Invest. 96:78-87); 2) exogenous HDL subfractions or reconstituted phospholipid-apoprotein complexes as described in Kritharides, et al. (1998 Arterioscler. Throm. Vasc. Biol. 18:1589-99); and 3) endogenously formed apoE as described by Huang, et al. (2001 Arterioscier. Throm. Vasc. Biol. 21:2019-25). In addition, RCT in living humans can be determined by measurement of fecal sterol excretion (Eriksson, et al., 1999 Circulation 100:594-8).

RCT functional assays can be performed using cells expressing LXRLI1 at a high level contacted with individual compounds or preparations containing different compounds. A preparation containing different compounds where one or more compounds affect RCT in cells over producing LXRLI1 as compared to control cells containing expression vector lacking LXRLI1 coding sequence, can be divided into smaller groups of compounds to identify the compound(s) affecting LXRLI1 mediated RCT activity.

LXRLI1 and RCT functional assays can be performed using recombinantly produced LXRLI1 present in different environments. Such environments include, for example, cell extracts and purified cell extracts containing the LXRLI1 expressed from recombinant nucleic acid and an appropriate membrane for the polypeptide; and the use of a purified LXRLI1 produced by recombinant means that is introduced into a different environment suitable for measuring RCT.

### MODULATING LXRLI1 EXPRESSION

LXRLI1 expression can be modulated as a means for increasing or decreasing LXRLI1 activity. Such modulation includes inhibiting LXRLI1 nucleic acid activity to reduce LXRLI1 expression or supplying LXRLI1 nucleic acid to increase LXRLI1 activity.

Inhibition of LXRLI1 Activity

LXRLI1 nucleic acid activity can be inhibited using nucleic acids recognizing LXRLI1 nucleic acid and affecting the ability of such nucleic acid to be transcribed or translated. Inhibition of LXRLI1 nucleic acid activity can be used, for example, in target validation studies.

A preferred target for inhibiting LXRLI1 translation is mRNA. The ability of mRNA encoding LXRLI1 to be translated into a protein can be effected by compounds such as anti-sense nucleic acid, RNA interference (RNAi) and enzymatic nucleic acid.

Anti-sense nucleic acid can hybridize to a region of a target mRNA. Depending on the structure of the anti-sense nucleic acid, anti-sense activity can be brought about by different mechanisms such as blocking the initiation of translation, preventing processing of mRNA, hybrid arrest, and degradation of mRNA by RNAse H activity.

RNAi also can be used to prevent protein expression of a target transcript. This method is based on the interfering properties of double-stranded RNA derived from the coding regions of gene that disrupts the synthesis of protein from transcribed RNA.

Enzymatic nucleic acid can recognize and cleave another nucleic acid molecule. Preferred enzymatic nucleic acids are ribozymes.

General structures for anti-sense nucleic acids, RNAi and ribozymes, and methods of delivering such molecules, are well known in the art. Modified and unmodified nucleic acids can be used as anti-sense molecules, RNAi and ribozymes. Different types of modifications can affect certain anti-sense activities such as the ability to be cleaved by RNAse H, and can effect nucleic acid stability. Examples of references describing different anti-sense molecules, and ribozymes, and the use of such molecules, are provided in U.S. Pat. Nos. 5,849,902; 5,859,221; 5,852,188; and 5,616,459. Examples of organisms in which RNAi has been used to inhibit expression of a target gene include: C. elegans (Tabara, et al., 1999 Cell 99:123-32; Fire, et al., 1998 Nature 391:806-11), plants (Hamilton and Baulcombe, 1999 Science 286:950-52), Drosophila (Hammond, et al., 2001 Science 293:1146-50; Misquitta and Patterson, 1999 Proc. Nat. Acad. Sci. 96:1451-56; Kennerdell and Carthew, 1998 Cell 95:1017-26), and mammalian cells (Bernstein, et al., 2001 Nature 409:363-6; Elbashir, et al., 2001 Nature 411:494-8).

Guidelines for pharmaceutical administration in general are provided in, for example, Remington's Pharmaceutical Sciences, 18^{th} Edition, supra, and Modern Phannaceutics, 2^{nd} Edition; supra. Nucleic acid-can be introduced into cells present in different environments using in vitro, in vivo, or ex vivo techniques.

Increasing LXRLI1 Expression

Nucleic acid coding for the LXRLI1 can be used, for example, to cause an increase in RCT or to create a test system (e.g., a transgenic animal) for screening for compounds affecting LXRLI1 expression. Nucleic acids can be introduced and expressed in cells present in different environments.

Guidelines for pharmaceutical administration in general are provided in, for example, Remington's Pharmaceutical Sciences, 18^{th} Edition, supra, and Modern Pharmaceutics, 2^{nd} Edition, supra. Nucleic acid can be introduced into cells present in different environments using in vitro, in vivo, or ex vivo techniques. Examples of techniques useful in gene therapy are illustrated in Gene Therapy & Molecular Biology: From Basic Mechanisms to Clinical Applications, Ed. Boulikas, Gene Therapy Press, 1998.

### GENE EXPRESSION PROFILING

Comparing patterns of gene expression is a widely used means of identifying novel genes, investigating gene function and finding potential new therapeutic targets (Shiue, et al., 1997 Drug Devel. Res. 41:142-59). The study of gene expression changes has played a major role in development of our understanding of nuclear receptor proteins. With the completion of the human genome sequencing effort, it is now a realistic goal to document all gene expression changes that occur during nuclear receptor ligand activation, in particular LXR. Historically, many techniques have been used to identify and clone differentially expressed genes (Liang, et al., 1992 Science 257:967-71; Welsh, et al., 1992 Nucleic Acids Res. 20:4965-70; Tedder, et al., 1988 Proc. Natl. Acad. Sci. 85:208-12; Davis, et al., 1984 Proc. Natl. Acad. Sci. 81:2194-8; Lisitsyn, et al., Science 259:946-51 (1993); Velculescu, et al., 1995 Science 270:484-7; Diatchenko, et al., 1996 Proc. Natl. Acad. Sci. 93:6025-30; Jiang, et al., 2000 Proc. Natl. Acad. Sci. 97:12684-9; Yang, et al., 1999 Nucleic Acids Res. 27: 517-23). However, these are generally not well suited for discerning the functional significance of gene expression differences. In many cases, these differences are not unique to a particular cellular pathway and the specificity of these changes becomes apparent only after secondary characterization using labor intensive techniques (Shiue, et al., 1997 Drug Devel. Res. 41:142-59).

Recently, it has become routine to use the technique of DNA microarray hybridization to quantify the expression of many thousands of discrete mRNA sequences in a single assay known as expression profiling (van't Veer, et al., 2002 Nature 415:530-36; Hughes, et al., 2001 Nature Biotech. 19:342-7; Hughes; et al., 2000, Cell 102:109-26; Lockhart and Winzeler, 2000 Nature 405:827-36; Roberts, et al., Science 2000 287:873-80; Wang et al., 1999 Gene 229:101-8; Lockhart, et al., 1996 Nat. Biotechnology 14:1675-80; Lockhart et al., U.S. Pat. No. 6,040,138; and Schena et al., 1995 Science 270: 467-70). Many applications have been described for expression profiling, but perhaps most relevant to elucidating gene function is the development of tools used to group genes according to similarities in patterns of gene expression in expression profiling experiments.

Coexpression of genes of known function with poorly characterized or novel genes has been suggested as a method to assign function to genes for which information is not available (Eisen, et al., 1998 Proc. Natl. Acad. Sci. 95:14863-8). Using a reference database or compendium of expression profiles from Saccharomyces cerevisiae, novel open reading frames (ORFs) showed that coordinated transcriptional regulations were enriched for a given phenotype, although there was potential for false positive detections (Hughes et al., 2000 Cell 102:109-126). In human cells, coregulation of uncharacterized expressed sequence tag (EST) sequences with known genes was noted, but no evaluation of the identities and properties of these ESTs was made.

In addition, expression profiles can also be used to identify pathway-specific reporters and target genes for a particular biological pathway of interest (see for example, WO 00/58520 and WO 00/58521). Such reporter genes, and probes directed to them, can be used to measure the activity of a particular biological pathway and may be further used in the design of drugs, drug therapies or other biological agents to target a particular biological pathway. Expression profiles can also be used to determine protein activity levels of a target protein using the methods described in U.S. Pat. No. 6,324,479.

The measurement of expression profiles using microarrays also has many important applications to the monitoring of disease states and therapies (see, for example, U.S. Pat. Nos. 6,218,122 and 6,222,093), and the identification of drug targets, identification of pathways of drug action and drug design (See, for example, U.S. Pat. No. 6,303,291; U.S. Pat. No. 6,165,709; U.S. Pat. No. 6,146,830; U.S. Pat. No. 5,965,352; and U.S. Pat. No. 5,777,888). As disclosed in many of the above-cited references, the functional identity of each gene represented on a microarray need not be understood in order for the overall expression profile to have a specific and substantial utility.

For example, van't Veer, et al. (2002 Nature 415:530-536) identified “good prognosis” and “poor prognosis” expression signatures that could be used to predict the clinical outcome of breast cancer patients. The function of many of proteins encoded by the genes represented in these expression signature groups remains unknown, yet the expression level of these genes within an expression signature still has utility in predicting disease outcome.

Similarly, U.S. Pat. No. 5,777,888 discloses the utility of microarray expression profiles to evaluate the target-specificity of a candidate drug by comparison of an expression profile obtained from cells treated with the candidate drug to a database of expression profiles obtained from cells treated with known drugs. Again, the function of a gene represented in these drug evaluation expression profiles need not be known in order to obtain useful information regarding the specificity of the candidate drug.

U.S. Pat. No. 6,218,122 provides methods for monitoring the disease state of a subject and determining the effect of a therapy upon a subject through the use of expression profiles wherein the functional identity of each gene represented in the expression profile need not be known to obtain the desired information (see also U.S. Pat. No. 6,266,093).

Shoemaker, et al. (2000 Nature 409:922-7; see also copending and commonly owned U.S. patent application Ser. No. 09/724,538 filed Nov. 11, 2000), provides methods for using microarray gene expression profiles to engage in gene discover and the detection of splice variants. Thus, microarrays comprising nucleic acids probes complementary and hybridizable to one or more exon sequences of the LXRLI1 coding sequence can be used to measure the expression levels of a plurality of LXRLI1 exons to permit high-throughput detection of splice variants of the LXRLI1 nucleic acids.

Markers Useful in Monitoring LXR Activity

Polynucleotides that are complementary and hybridizable to the LXR-regulated markers listed in Table 1 can be used to select LXR pathway reporters using the methods described in WO 00/58520 and WO 00/58521. LXR pathway reporters are useful, for example, in gene expression assays to diagnose and monitor treatment of diseases related to cholesterol metabolism and to identify and classify compounds affecting LXR activity. LXR pathway reporters can also be used to identify and categorize cholesterol disease subtypes not heretofore appreciated using conventional diagnostic tests. Expression of the LXR pathway reporter genes of the present invention can be measured using any of a variety of methods well known in the art, such as Northern blots, microarrays, and RT-PCR, and the like.

Another aspect of the present invention provides a method of estimating LXR activity in a subject. The same method can also be used to detect changes in LXR activity in a sample in response to a disease state or treatment of a disease. The method comprises the steps of measuring a transcript level in a sample of mRNA or nucleic acid derived therefrom from the subject, wherein the transcript comprises a nucleic acid sequence selected from the sequence identification numbers listed in Table 3. The measured level of the selected transcript is then compared to the level of the same transcript measured in a control sample. In another embodiment, the method additionally includes the measurement of a transcript comprising a nucleotide sequence selected from the sequence identification numbers listed in Table 2. In alternative embodiment, the transcript encodes a polypeptide comprising an amino;:acid sequence selected from the group consisting of SEQ ID NO 2, SEQ ID NO 14, SEQ ID) NO 16, SEQ ID NO 18, SEQ ID NO 20, SEQ-ID NO 22, SEQ ID NO 24, SEQ ID NO 26, SEQ ID NO 28, SEQ ID; NO 30, SEQ ID NO 32, SEQ ID NO 34, SEQ ID NO 36, SEQ ID NO 38, SEQ ID NO 40, SEQ ID NO 42, SEQ ID NO 44, SEQ ID NO 46, SEQ ID NO 48, SEQ ID NO 50, SEQ ID NO 52, SEQ ID NO 54, SEQ ID NO 56, SEQ ID NO 58, SEQ ID NO 60, SEQ ID NO 62, SEQ ID NO 64, SEQ ID NO 66, SEQ ID NO 68, SEQ ID NO 70, SEQ ID NO 72, SEQ ID NO 75, SEQ ID NO 78, SEQ ID NO 80, SEQ ID NO 83, and SEQ ID NO 96.

As would be apparent to an artisan of biological assays, a variety of different kinds of control samples may be used to practice the above-described methods for estimating LXR activity or detecting changes in LXR activity in a sample. For example, LXR activity may be estimated by reference to a control sample obtained from cells that express LXR gene at a low level due to, for example, the presence of a mutation in the LXR gene or due to inhibition of LXR expression by using RNAi. Other techniques for obtaining cells that express LXR at a low level are also well known in the art. Alternatively, the control sample may be obtained from cells that express high levels of LXR. Again, a variety of methods are well known in the art for manipulating the expression of a gene of interest. In some embodiments of the method, multiple control samples are used to produce a graph that relates the level of LXR expression in a cell sample to the level of expression of the measured transcripts. Alternatively, multiple control samples can be made by treating a series of cell samples from a subject with an increasing amount of a LXR agonist compound, such as APD, and measuring transcript levels corresponding to any of the markers listed in Tables 2 and 3.

In other embodiments, the methods described above for measuring LXR activity are used to diagnose a disease or disorder involving LXR activity in a sample by detecting an increase or decrease in the measured transcript level relative to the amount of the same transcript present in an analogous sample from a subject not having the disease or disorder or not subjected to therapy. Preferably, the disease or disorder is cholesterol gallstones, atherosclerosis, lipid storage diseases, obesity, diabetes, or hypercholesterolemia.

In another embodiment the methods of measuring LXR activity are used to screen for a compound that changes the activity of LXR, wherein the compound involves increased or decreased level of LXR activity in a sample from the subject contacted with the compound relative to the level present in an analogous sample from the subject not contacted with the compound. Preferably, the compound is a LXR ligand, more preferably an agonist.

The invention further provides gene marker sets that distinguish various cholesterol metabolic states of a biological sample and methods of use therefor. In one embodiment, the invention provides a method for determining the cholesterol metabolic state of a biological sample comprising detecting a difference in the expression of a first plurality of genes relative to a control biological sample, the first plurality of genes consisting of LXRLI1 and at least one of the genes corresponding to the markers listed in Table 1, excluding LXRLI1. In another embodiment, the first plurality of genes consists of at least two or more of the genes corresponding to the markers listed in Table 3. In yet another embodiment, the first plurality of genes consists of at least one or more of the genes corresponding to the markers listed in Table 3 and one or more of the genes corresponding to the markers listed in Table 2.

The invention further provides a method for classifying a test LXR ligand as a full LXR-ligand or as a selective LXR-ligand. The method comprising detecting a difference in the expression of a plurality of genes in a first cell sample contacted by the test LXR-ligand compared to the expression of the plurality of genes in a second cell sample contacted by a reference LXR-ligand, the plurality of genes consisting of at least five genes corresponding to markers listed in Table 1, wherein the test LXR-ligand is classified as a full activity LXR-ligand if each gene in said plurality of genes is similarly regulated in the first and second cell samples, and, the test LXR-ligand is classified as a selective activity LXR-ligand if fewer than all of the genes in the plurality of genes are similarly regulated in the first cell sample as compared to the second cell sample. Preferably, the ligand is an agonist and the plurality of genes consists of all of the genes corresponding to markers listed in Table 1. In other embodiments the plurality of genes consists of at least 10, 15, 20, 25, 30, 35, 40, 45, 50 or all of the markers listed in Table 1. Preferable, in each case, at least one gene marker is selected from Group I genes, as defined in FIG. 2, and at least one gene marker is selected from Group II genes, as defined in FIG. 2.

### EXAMPLES

Examples are provided below to further illustrate different features and advantages of the present invention. The examples also illustrate useful methodology for practicing the invention. These examples do not limit the claimed invention.

### Example 1

Identification of LXR Regulated Markers Using Microarray Expression Profiles

To identify human transcripts up or down regulated by LXR-ligand, FlexJet™ microarrays representing either 25,000 Unigene clusters were hybridized to a mixture of cRNAs prepared from samples obtained from untreated versus treated cells of various types. Microarrays, and materials and methods for preparing hybridization samples from purified RNA, hybridizing the microarrays, detecting hybridization signals, and data analysis are described in van't Veer,iet al. (2002 Nature 415:530-536) and Hughes, et al. (2001 Nature Biotechnol. 19:342-7).

Experiments were conducted comparing gene expression profiles of primary hepatocytes, primary human macrophage and THP-1 cells treated with a variety of compounds known to activate the LXR and PPAR nuclear receptor regulated pathways. Additional compound treatments were performed which are known to cause an increase, or decrease in cellular cholesterol. All cells were cultured at 37° C. in a humidified atmosphere consisting of 95% air and 5% carbon dioxide. For each cell sample, mRNA was extracted and purified using Trizol reagent (Invitrogen Corporation, Carlsbad, Calif.) followed by DNAse treatment, as described by Fu, et al. (2001 J. Biol. Chem. 276:38378-87). For the primary hepatocytes, the Trizol purification was followed by a RNasy DNAase (Promega Corp, Madison, Wis.) step per the manufacturer's instructions. Purified mRNA samples were processed for microarray hybridizations as described in van't Veer, et al. (2002 Nature 415:530-536). Specific cell types were grown and treated with compounds as described below.

THP-1 cells were obtained from ATCC (TIB-202) and were grown in Medium A (RPMI-1640 medium (Sigma Cat.#R8005) containing 10 mM HEPES buffer, 10% fetal calf serum (FCS), 1 mM sodium pyruvate, 2 mM L-Glutamine, and Antibiotic-Antimycotic Solution (Sigma Cat.#A9909, 100 U/ml Penicillin, 0.1 mg/ml Streptomycin, 0.25 mg/ml Amphotericin B)). THP-1 cells were differentiated into macrophages in 6-well tissue culture dishes at a density of 3 million cells/well by incubation in the same medium plus 100 nM phorbol 12-Myristate 13-acetate (Sigma Cat.#P8139) for three days. After differentiation into macrophages, cells were collected and, further treated as described below and in figure legends for each specific compound.

Human primary monocytes were prepared as described by Wright and Silverstein (1982 J. Exp. Med. 156:1149-64), and differentiated to macrophages by culturing for 7 days in Teflon jars in RPMI-1640 medium (VWR International, West Chester, Pa.) supplemented with 12% human serum, 100 units/ml penicillin and 100 mg/ml streptomycin sulfate). Cells were then plated in the same medium to initiate compound exposure as described below for each specific compound. At the time intervals indicated in figure legends, cell samples were collected and mRNA extracted. Extracted mRNA samples were prepared for microarray hybridizations as described in van't Veer, et al. (2002 Nature 415:530-536).

Human primary hepatocytes were received from In Vitro Technologies (Baltimore, Md.) in six well dishes and maintained in phenol red-free DMEM (high glucose) containing 10% charcoal-stripped FCS (Geminni Bio-Products, Inc., Calabasas, Calif.), 1% nonessential amino acids, 1% glutamine and 100 units/ml Penicillin G and 100 μg/ml Streptomycin sulfate. Dexamethasone (Sigma-Aldrich Corp., St. Louis, Mo.) was also added to the growth medium at 0.01 μM to maintain hepatocyte viability. Cells were then plated in the same medium to initiate compound exposure experiments using the compounds and concentrations indicated below and in the figure legends. Cell samples were collected after 24 hours of compound exposure and mRNA extracted.

Acetyl-podocarpic dimer, zaragozic acid, lovastatin and fenofibrate were synthesized at the Merck Research Laboratories (Rahway, N.J.). Glucagon was purchased from Sigma-Aldrich Corp. Acetylated low density lipoprotein (AcLDL) was purchased from Intracel Corp (Rockville Md.). Methyl-β-Cyclodextrin (CD), Cholesterol (Ch) and 22(R)-hydroxycholesterol were purchased from Sigma-Aldrich). Cells were treated with test compounds in the following fashion:

Acetyl-Podocarpic Dimer (APD) is a highly specific LXR agonist (Sparrow, et al., 2002 J. Biol. Chem. 10.1074/jbc.M1108225200). Hepatocytes were treated with APD at 0.001 μM, 0.01 μM and 0.1 μM for 24 hours, followed by a reapplication of fresh media containing compound for an additional six hours prior to extraction of total RNA. Macrophages were treated with APD at 0.1 μM for 24 hours prior to extraction of total RNA. THP-1 cells were treated with APD at 0.1 μM for 3, 6, or 20 hours prior to extraction of total RNA.

Dexamethasone is an anti-inflammatory glucocorticoid receptor ligand. For experiments involving the use of primary hepatocyte cells, dexamethasone was added to the culture medium at 0.01 μM to enhance hepatocyte viability.

Fenofibrate is a PPARa agonist (Guo, et al., 2001 Biochim. Biophys. Acta. 1533:220-32). Hepatocyte cell samples were treated with fenofibrate at 0.01 mM, 0.03 mM and 0.1 mM for 24 hours, then the medium replaced with fresh media containing fenofibrate at the indicated concentration and the cells incubated for an additional six hours prior to extraction of total RNA.

Glucagon is a glucagon receptor agonist (Houslay, et al., 1976 Biochim. Biophys. Acta. 436:495-504; Broer, et al., 1977 Endocrinology 101:236:49). Hepatocyte cell samples were treated with glucagon at 0.01 μM for 24 hours, then the medium was replaced with fresh media containing 0.01 μM glucagon and the cells incubated for an additional six hours prior to extraction of total RNA.

Lovastatin is an inhibitor of HMG-CoA reductase that reduces cholesterol and isoprenoid synthesis (Alberts, et al., 1980 Proc. Natl. Acad. Sci. 77:3957-61). THP-1 cell samples were treated with 50 μM lovastatin in DMSO for 3 hours, 6 hours and 20 hours, respectively, prior to extraction of total RNA.

Zaragozic Acid is an inhibitor of squalene synthase which results in inhibition of cholesterol synthesis, but not the synthesis of other isoprenoids (Bergstrom et al., 1993 Proc. Natl. Acad. Sci. 90:80-4). THP-1 cells were treated with 100 nM Zaragozic acid in DMSO for 3 hours 6 hours and 20 hours, respectively, prior to extraction of total RNA.

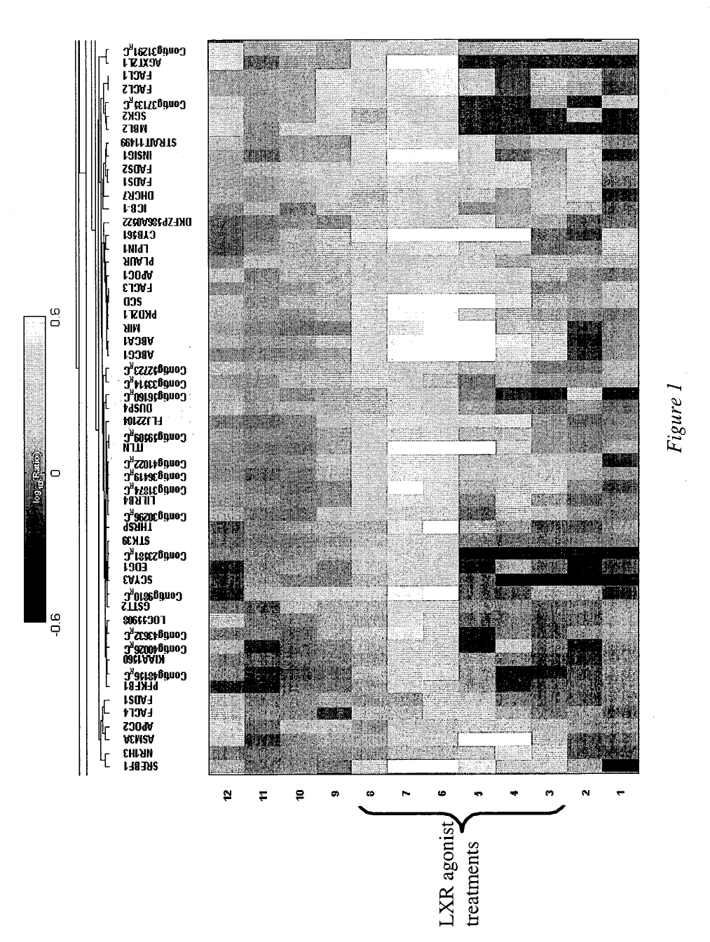

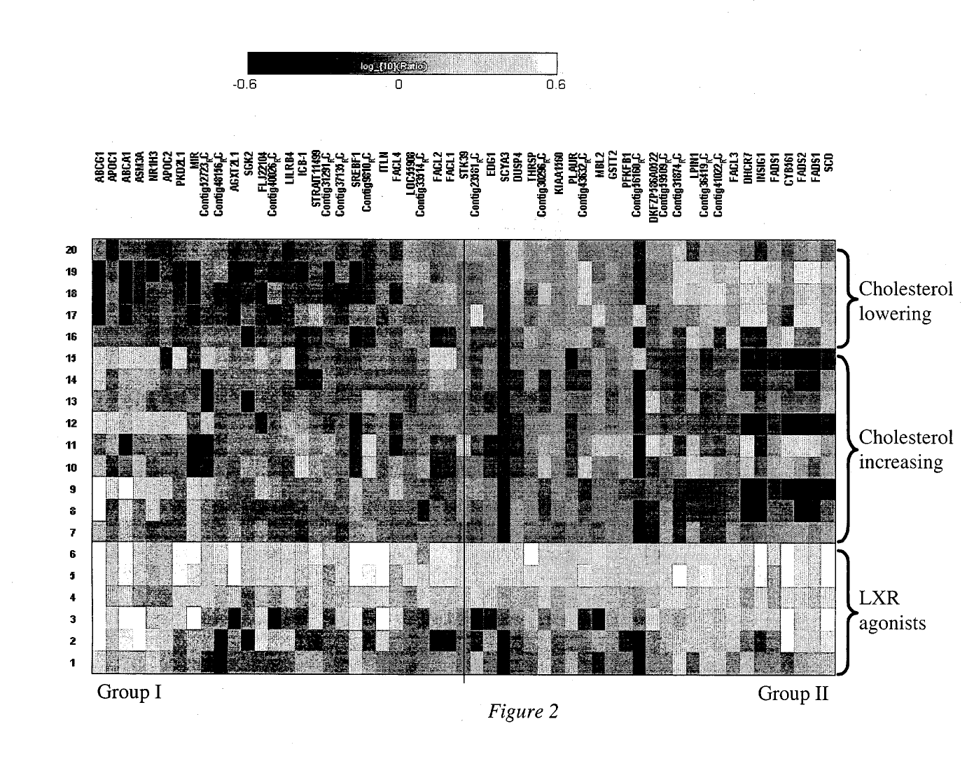

A total of 297 markers exhibited changes in the log ratio of expression of >0.2, with a P-value of <0.01 in at least two of the total of twelve experiments which were analyzed. The gene expression profiles of the 297 markers over the twelve experiments were analyzed by using a two dimensional hierarchical clustering algorithm. This cluster analysis groups genes showing the greatest similarity of regulation over all experiments (first dimension) and the experiments showing the greatest similarities in gene regulation (second dimension). For clarity, FIG. 1 displays only a section (55 markers) of the total data set. Each experiment and each marker, including gene names when known, are represented on the X- and Y-axes, respectively. Genes up-regulated in a particular experiment are colored dark gray; genes down regulated in that experiment are colored light gray; and genes showing no regulation in a particular experiment are colored black. The set of markers shown in FIG. 1 identifies genes that are transcriptionally up-regulated by compounds that activate LXR.

Table 1 presents a list of all of the markers (and corresponding nucleic acid and amino acid SEQ ID NOs) corresponding to the LXR-ligand induced transcripts identified from FIG. 1. Hereafter the markers listed in Table 1 are collectively referred to as the “LXR-regulated geneset.” Of the 55 markers within the LXR-regulated geneset, five markers represented genes known to be LXR regulated and to be involved with cholesterol and/or lipid metabolism. These five previously known LXR regulated genes are listed in Table 2.

Table 3 lists 50 markers identified in FIG. 1 that were not previously known to be regulated by LXR, but were identified as such in this microarray data set. Of these 50 markers, 32 markers corresponded to genes having some level of functional annotation. Inspection of the putative functions of the proteins encoded by the 32 gene markers in Table 3 revealed that the most prevalent shared functional characteristic of the group was that 14 of the proteins are reasonably involved with, or effect, cholesterol and/or lipid metabolism. Six of the markers listed in Table 3, including an incomplete, truncated version of LXRLI1 (reference as NM_{—}022918), encoded computationally predicted proteins whose functions have not yet been identified, and 13 markers represented ESTs that have not yet been experimentally associated to a gene sequence.

Inspection of NM_{—}022918 and predicted protein FLJ22104 sequences indicated that the predicted coding region was probably incomplete. On this basis NM_{—}022918 was selected from the set of markers listed in Table 3 and a full length cDNA clone was isolated and its nucleotide sequence determined as described in Example 2 below.

TABLE 1

55 markers representing the LXR-regulated geneset
and corresponding nucleotide and protein sequence reference numbers.

| Systematic | Nucleotide | Amino Acid | Systematic | Nucleotide | Amino Acid |
|---|---|---|---|---|---|
| Name | Sequence | Sequence | Name | Sequence | Sequence |
| LXRLII | SEQ ID NO 1 | SEQ ID NO 2 | NM_000242 | SEQ ID NO 57 | SEQ ID NO 58 |
| NM_005502 | SEQ ID NO 3 | SEQ ID NO 4 | NM_016112 | SEQ ID NO 59 | SEQ ID NO 60 |
| NM_004176 | SEQ ID NO 5 | SEQ ID NO 6 | NM_017625 | SEQ ID NO 61 | SEQ ID NO 62 |
| NM_005063 | SEQ ID NO 7 | SEQ ID NO 8 | NM_013262 | SEQ ID NO 63 | SEQ ID NO 64 |
| NM_004915 | SEQ ID NO 9 | SEQ ID NO 10 | NM_002625 | SEQ ID NO 65 | SEQ ID NO 66 |
| NM_005693 | SEQ ID NO 11 | SEQ ID NO 12 | NM_001394 | SEQ ID NO 67 | SEQ ID NO 68 |
| NM_001995 | SEQ ID NO 13 | SEQ ID NO 14 | NM_002983 | SEQ ID NO 69 | SEQ ID NO 70 |
| D10040 | SEQ ID NO 15 | SEQ ID NO 16 | NM_001400 | SEQ ID NO 71 | SEQ ID NO 72 |
| NM_004457 | SEQ ID NO 17 | SEQ ID NO 18 | NM_018687 | SEQ ID NO 73 | |
| NM_004458 | SEQ ID NO 19 | SEQ ID NO 20 | NM_002659 | SEQ ID NO 74 | SEQ ID NO 75 |
| NM_013402 | SEQ ID NO 21 | SEQ ID NO 22 | AK022997 | SEQ ID NO 76 | |
| AF035284 | SEQ ID NO 23 | SEQ ID NO 24 | AK056513 | SEQ ID NO 77 | SEQ ID NO 78 |
| NM_004265 | SEQ ID NO 25 | SEQ ID NO 26 | AJ272057 | SEQ ID NO 79 | SEQ ID NO 80 |
| NM_001645 | SEQ ID NO 27 | SEQ ID NO 28 | Contig59509_RC | SEQ ID NO 81 | |
| NM_000483 | SEQ ID NO 29 | SEQ ID NO 30 | AF113007 | SEQ ID NO 82 | SEQ ID NO 83 |
| NM_001360 | SEQ ID NO 31 | SEQ ID NO 32 | Contig9810_RC | SEQ ID NO 84 | |
| AB046780 | SEQ ID NO 33 | SEQ ID NO 34 | Contig23581_RC | SEQ ID NO 85 | |
| NM_003251 | SEQ ID NO 35 | SEQ ID NO 36 | Contig30296_RC | SEQ ID NO 86 | |
| D80010 | SEQ ID NO 37 | SEQ ID NO 38 | Contig31291_RC | SEQ ID NO 87 | |
| BC018999 | SEQ ID NO 39 | SEQ ID NO 40 | Contig31874_RC | SEQ ID NO 88 | |
| U06715 | SEQ ID NO 41 | SEQ ID NO 42 | Contig33514_RC | SEQ ID NO 89 | |
| NM_005542 | SEQ ID NO 43 | SEQ ID NO 44 | Contig36419_RC | SEQ ID NO 90 | |
| NM_031279 | SEQ ID NO 45 | SEQ ID NO 46 | Contig37135_RC | SEQ ID NO 91 | |
| NM_000854 | SEQ ID NO 47 | SEQ ID NO 48 | Contig41022_RC | SEQ ID NO 92 | |
| NM_013233 | SEQ ID NO 49 | SEQ ID NO 50 | Contig43632_RC | SEQ ID NO 93 | |
| NM_016276 | SEQ ID NO 51 | SEQ ID NO 52 | Contig48156_RC | SEQ ID NO 94 | |
| NM_006847 | SEQ ID NO 53 | SEQ ID NO 54 | AF289609 | SEQ ID NO 95 | SEQ ID NO 96 |
| NM_004848 | SEQ ID NO 55 | SEQ ID NO 56 | | | |

TABLE 2
Genes listed in Table 1, that were previously known to be regulated by activated
LXR.

| | | Protein | |
|---|---|---|---|
| Systematic Name | Gene Name | Description | Gene Category |
| NM_005502 | ABCA1 | ATP-binding cassette, sub- | ATP-binding, Glycoprotein, |
| | | family A (ABC1), member 1. | Transmembrane, Transport, |
| | | LXR Regulated: J. Biol. | Cholesterol metabolism, |
| | | Chem. 2000, 275:28240-45. | Atherosclerosis, Disease |
| | | | mutation, Polymorphism |
| NM_004176 | SREBF1 | Sterol regulatory element | Transcription regulation, |
| | | binding transcription factor 1 | Activator, DNA-binding, |
| | | LXR Regulated: Gene & | Lipid metabolism, |
| | | Development, 2000, | Cholesterol metabolism, |
| | | 14:2819-30. | Nuclear protein, |
| | | | Transmembrane, |
| | | | Endoplasmic reticulum, |
| | | | Golgi stack, Alternative |
| | | | splicing, 3D-structure |
| NM_005063 | SCD | Stearoyl-CoA desaturase | Oxidoreductase, Fatty acid |
| | | (delta-9-desaturase) | biosynthesis, |
| | | LXR Regulated: Gene & | Transmembrane, |
| | | Development, 2000, | Endoplasmic reticulum, Iron, |
| | | 14:2831-8. | Hypothetical protein |
| NM_004915 | ABCG1 | ATP-binding cassette, sub- | ATP-binding, |
| | | family G (WHITE), | Transmembrane, Transport, |
| | | member 1. | Alternative splicing, |
| | | LXR Regulated: J. Biol. | Alternative initiation, |
| | | Chem. 2001, 276:39438-47. | Polymorphism |
| NM_005693 | NR1H3 (LXRα) | Nuclear receptor subfamily 1, | Receptor, Transcription |
| | | group H, member 3 | regulation, DNA-binding, |
| | | LXR Regulated: Mol. Cell. | Nuclear protein, Zinc-finger |
| | | Biol. 2001, 21:7558-68. | |

TABLE 3
Genes listed in Table 1, that were first identified in this work as being regulated by
LXR.

| | | Protein | |
|---|---|---|---|
| Systematic Name | Gene Name | Description | Gene Category |
| NM_001995 | FACL1 | Fatty-acid-Coenzyme A | Ligase, Fatty acid |
| | | ligase, long-chain 1 | metabolism, Magnesium, |
| | | | Multigene family |
| D10040 | FACL2 | Fatty acid Coenzyme A | Ligase, Fatty acid |
| | | ligase, long chain 2 | metabolism, Magnesium, |
| | | | Multigene family |
| NM_004457 | FACL3 | Fatty-acid-Coenzyme A | Ligase, Fatty acid |
| | | ligase, long-chain 3 | metabolism, Magnesium, |
| | | | Multigene family |
| NM_004458 | FACL4 | Fatty-acid Coenzyme A | Ligase, Fatty acid |
| | | ligase, long-chain 4 | metabolism, Magnesium, |
| | | | Multigene family, |
| | | | Alternative splicing |
| NM_013402 | FADS1 | Fatty acid desaturase 1 | Heme, Fatty acid |
| | | | metabolism |
| AF035284 | FADS1 | Fatty acid desaturase 1 | Heme, Fatty acid |
| | | | metabolism |
| NM_004265 | FADS2 | Fatty acid desaturase 2 | Heme, Fatty acid |
| | | | metabolism, Hypothetical |
| | | | protein |
| NM_001645 | APOC1 | Apolipoprotein C-I, | Plasma, Lipid transport, |
| | | transgenic mice have | VLDL, Signal, 3D-structure, |
| | | hypertriglyceridemia (2001 | Lipoprotein |
| | | Diabetes 50:2779-85). | |
| NM_000483 | APOC2 | Apolipoprotein C-II | Chylomicron, VLDL, |
| | | | Plasma, Lipid transport, |
| | | | Lipid degradation, Signal, |
| | | | Disease mutation, |
| | | | Polymorphism, |
| | | | Hyperlipidemia, Lipoprotein |
| NM_001360 | DHCR7 | 7-Dehydrocholesterol | Sterol biosynthesis, |
| | | reductase | Cholesterol biosynthesis, |
| | | | Oxidoreductase, NADP, |
| | | | Transmembrane, |
| | | | Endoplasmic reticulum, |
| | | | Disease mutation |
| AB046780 | KIAA156O | Glycerol-3-phosphate | Phospholipid biosynthesis, |
| (referred to as Contig42768 | | acyltransferase | Transferase, Acyltransferase, |
| in FIGS. 1 and 2) | | | Transmembrane, |
| | | | Mitochondrion, Transit |
| | | | peptide |
| NM_003251 | THRSP | Thyroid hormone responsive | Fatty Acid Synthesis |
| | | (SPOT14 homolog, rat). | |
| | | Expressed in lactating | |
| | | mammary, adipose, and liver | |
| | | and activates genes encoding | |
| | | the enzymes of fatty acid | |
| | | synthesis (1998 PNAS | |
| | | 95:6989-94). | |
| D80010 | LPIN1 | Lipin 1, mouse mutation | Lipodystropy, Hypothetical |
| | | causes lipodystropy (2001 | protein |
| | | Nat. Gen. 27:121-4). | |
| BC018999 | ASM3A | Acid sphingomyelinase-like | Hydrolase, Glycosidase, |
| (referred to as AK000184 in | | phosphodiesterase. | |
| FIGS. 1 and 2) | | | |
| U06715 | CYB561 | Cytochrome b-561 is a major | Electron transport, |
| | | transmembrane protein of | Transmembrane, Heme, |
| | | catecholamine and | possible cofactor for FADS |
| | | neuropeptide secretory | |
| | | vesicles. | |
| NM_005542 | INSIG1 | Insulin induced gene 1 | Leucine-rich repeat, Repeat, |
| | | | Thought to play a role in |
| | | | growth and differentiation of |
| | | | tissues involved in metabolic |
| | | | control. |
| NM031279 | AGXT2L1 | Alanine-glyoxylate | Transferase, |
| (referred to as Contig31546 | | aminotransferase 2-like 1 | Aminotransferase |
| in FIGS. 1 and 2) | | | |
| NM_000854 | GSTT2 | Glutathione S-transferase | Transferase, Multigene |
| | | theta 2 | family, 3D-structure |
| NM_013233 | STK39 | Serine threonine kinase 39 | Transferase, |
| | | (STE20/SPS1 homolog, | Serine/threonine-protein |
| | | yeast) | kinase, ATP-binding |
| NM_016276 | SGK2 | Serum/glucocorticoid | ATP-binding, Kinase, |
| | | regulated kinase 2 | Serine/threonine-protein |
| | | | kinase, Transferase, |
| | | | Hypothetical protein |
| NM_006847 | LILRB4 | Leukocyte immunoglobulin- | Signal, Receptor |
| | | like receptor, subfamily B | |
| | | (with TM and ITIM | |
| | | domains), member 4 | |
| NM_004848 | ICB-1 | Basement membrane-induced | |
| | | gene | |
| NM_000242 | MBL2 | Mannose-binding lectin | Signal, Lectin, |
| | | (protein C) 2, soluble | Hydroxylation, |
| | | (opsonic defect) | Glycoprotein, Mannose- |
| | | | binding, Membrane, |
| | | | Calcium, Collagen, Repeat, |
| | | | Polymorphism, 3D-structure |
| NM_016112 | PKD2L1 | Polycystic kidney disease 2- | |
| | | like 1 | |
| NM_017625 | ITLN | Intelcetin | |
| NM_013262 | MIR | Myosin regulatory light chain | |
| | | interacting protein | |
| NM_002625 | PFKFB1 | 6-phosphofructo-2- | Multifunctional enzyme, |
| | | kinase/fructose-2,6- | Transferase, Kinase, |
| | | biphosphatase 1 | Hydrolase, ATP-binding, |
| | | | Phosphorylation, Liver, |
| | | | Multigene family |
| NM_001394 | DUSP4 | Dual specificity phosphatase | Hydrolase, Hypothetical |
| | | 4 | protein, Nuclear protein |
| NM_002983 | SCYA3 | Small inducible cytokine A3 | Cytokine, Chemotaxis, |
| | | | Inflammatory response, |
| | | | Signal |
| NM_001400 | EDG1 | Endothelial differentiation, | Receptor, Hypothetical |
| | | sphingolipid G-protein- | protein, G-protein coupled |
| | | coupled receptor, 1 | receptor, Transmembrane, |
| | | | Glycoprotein, |
| | | | Phosphorylation, |
| | | | Lipoprotein, Palmitate |
| NM_004458 | LOC55908 | Hepatocellular carcinoma- | |
| | | associated gene TD26 | |
| NM_002659 | PLAUR | plasminogen activator, | Receptor, Kinase, Signal, |
| | | urokinase receptor | Glycoprotein, GPI-anchor, |
| | | | Repeat, Alternative splicing |
| LXRLI1 (This work, referred | LXRLI1 | LXRLI1 | LXR-ligand induced, |
| to as FLJ22104 in FIGS. 1 | | | predicted transmembrane |
| and 2) | | | protein |
| AK022997 | FLJ12935 | Homo sapiens cDNA | Hypothetical protein |
| (referred to as | | FLJ12935 tis, clone | |
| Contig40026_RC in FIGS. 1 | | NT2RP2004982 | |
| and 2) | | | |
| AK056513 | FL131951 | Homo sapiens cDNA | Hypothetical protein |
| (referred to as | | FLJ31951 fis, clone | |
| Contig52723_RC in FIGS. 1 | | NT2RP7007177, weakly | |
| and 2) | | similar to Homo sapiens | |
| | | multiple membrane spanning | |
| | | receptor TRC8 mRNA | |
| AJ272057 | STRAIT11499 | Hypothetical protein | Hypothetical protein |
| | | STRAIT11499 | |
| Contig59509_RC | | | |
| AF113007 | DKFZP586A0522 | DKFZP586A0522 protein, | Hypothetical protein |
| | | cDNA clone from human | |
| | | fetal liver | |
| Contig9810_RC | ESTs | | |
| Contig23581_RC | ESTs | | |
| Contig30296_RC | ESTs | | |
| Contig31291_RC | ESTs | | |
| Contig31874_RC | ESTs | | |
| Contig33514_RC | ESTs | | |
| Contig36419_RC | ESTs | | |
| Contig37135_RC | ESTs | | |
| Contig41022_RC | ESTs | | |
| Contig43632_RC | ESTs | | |
| Contig48156_RC | ESTs | | |
| AF289609 | ESTs | | |
| (referred to as | | | |
| Contig56160_RC in FIGS. 1 | | | |
| and 2) | | | |

### Example 2

Cloning and Sequencing of LXRLI1

Microarray data indicated that mRNA NM_{—}022918 (SEQ ID NO 97), encoding the predicted protein FU22104 (SEQ ID NO 98) was induced in LXR-ligand experiments (see FIG. 1). Mouse homology to FU22104 indicated that there might be 82 amino acids more to be added to the amino terminal end of FLJ22104 ORF (mRNA NM_{—}022918). The new predicted ORF was a combination of EST BM149697 (SEQ ID NO 99) and overlapping mRNA NM_{—}022918. The new predicted ORF protein was named LXRLI1 and the encoding nucleotide region was named LXRLI1. RT-PCR primers were designed to include, not only the new 5′ extension of the open reading frame contained within BM149697, but also noncoding sequence in the genomic sequence 5′ to BM149697. This 5′ genomic sequence contained an in-frame STOP codon. The presence of this in-frame STOP codon 5′ to the LXRLI1 ORF indicates that the predicted ORF is full length and cannot be extended beyond the predicted starting methionine amino acid. The 5′ “forward” primer used to amplify and clone the entire LXRLI1 ORF was designed to have a nucleotide of 5′ CAGAGTAACCCCGCTCTCGTGAC 3′ (SEQ ID NO 100). The 3′ “reverse” primer was designed to have the nucleotide sequence of 5′ GTGTTCAACATAATTAACTCTTCA AGATTG 3′ (SEQ ID NO 101). All PCR and sequencing primers were obtained from RESGEN/Invitrogen, Huntsville, Ala.

RT-PCR

The LXRLI1 cDNA sequence was cloned using a combination of RT and PCR. 20 ng of small intestinal mRNA (Ambion, Austen, Tex.) or 400 ng of THP-1 total RNA (see above for description of THP-1 cell line) was reverse transcribed using Superscript II (Gibco/Invitrogen,, Carlsbad, Calif.) and oligod(T) primer (RESGEN/Invitrogen, Huntsville, Ala.) according to the Superscript II manufacturer's instructions. For PCR, 1 μl of the completed RT reaction was added to 40 μl of water, 5 μl of 10× buffer, 1 μl of dNTPs and 1 μl of enzyme from the Clonetech (PaloAlto, Calif.) Advantage 2 PCR kit. PCR was done in a Gene Amp PCR System 9700 (Applied Biosystems, Foster City, Calif.). After an initial 94° C. denaturation of 1 minute, 35 cycles of a 30 second denaturation at 94° C. followed by a 1 minute annealing at 65° C. and a 90 second synthesis at 68° C. The 35 cycles of PCR were followed by a 7 minute extension at 68° C. The 50 μl reaction was then chilled to 4° C. 10 ml of the resulting reaction product was run on a 1% agarose (Invitrogen, Ultra pure) gel stained with 0.3 μg/ml ethidium bromide (Fisher Biotech, Fair Lawn, N.J.). The gel was visualized and photographed on a UV light box to determined if the PCR had yielded products of the expected size, in the case of the predicted LXRLI1, ORF about 1.5 kilobases (Kb). A faint about 1.5 Kb stained DNA band was seen for the RT-PCR from small intestine. The remainder of the 50 ml PCR reactions from small intestine and THP-1 cells was purified using the QIAquik Gel extraction Kit (Qiagen, Valencia, Calif.) following the QIAquik PCR Purification Protocol provided with the kit. An about 50 μl of product obtained from the purification protocol was concentrated to about 6 μl by drying in a Speed Vac Plus (SC110A, from Savant, Holbrook, N.Y.) attached to a Universal Vacuum Sytem 400 (also from Savant) for about 30 minutes on medium heat.

Cloning of RT-PCR Products

About 4 μl of the 6 μl of purified LXRLI1 RT-PCR product was used in a cloning reaction using the reagents and instructions provided with the TOPO TA cloning kit (Invitrogen, Carlsbad, Calif.). About 2 μg of the cloning reaction was used following the manufacturer's instructions to transform TOP10 chemically competent E. coli provided with the cloning kit. After the 1 hour recovery of the cells in SOC medium (provided with the TOPO TA cloning kit), 200 μl of the mixture was plated on LB medium plates (Sambrook, et al., in Molecular Cloning, A Laboratory Manual, 2^{nd} Edition, Cold Spring Harbor Laboratory Press, 1989) containing 100 μg/ml Ampicillin (Sigma, St. Louis, Mo.) and 80 μg/ml X-GAL (5-Bromo-4-chloro-3-indoyl B-D-galactoside, Sigma, St. Louis, Mo.). Plates were incubated overnight at 37° C. White colonies were picked from the plates into 2 ml of 2×LB medium. These liquid cultures were incubated overnight on a roller at 37° C. Plasmid DNA was extracted from these cultures using the Qiagen (Valencia, Calif.) Qiaquik Spin Miniprep kit. About 3 μl of each DNA miniprep was digested 1 hour at 37° C. with 0.5 μl of the restriction enzyme EcoRI (provided at 10 units/μl by Gibco/Invitrogen, Carlsbad, Calif.). About 10 μl of the 15 μl digestion reaction was run on a 1% Agarose gel and the DNA bands were visualized and photographed on a UV light box to determine which minipreps samples had inserts of the size predicted for a cDNA insert encoding for the predicted LXRLI1 ORF, about 1.5 Kb. Six clones having the expected 1.5 Kb inserts were identified and prepared for DNA sequencing of the putative LXRLI1 ORF.

Sequencing of RT-PCR Products from cDNA Clones

Six putative full length clones of LXRLI1 were chosen for sequencing (two clones obtained from small intestinal mRNA and four clones obtained from THP-1 total RNA). About 4 ml of each DNA miniprep (described previously above) were used in a DNA sequencing reaction with each of the following oligonucleotide primers:

| | |
|---|---|
| F | |
| 5′ GTAAAACGACGGCCAGT 3′ | (SEQ ID NO 102) |
| R | |
| 5′ GGAAACAGCTATGACCATG 3′ | (SEQ ID NO 103) |
| MB291 | |
| 5′ CCAAAGCCAGGAGTCCATGAGTAG 3′ | (SEQ ID NO 104) |
| MB306 | |
| 5′ TGGTCTAGTCAGGAAATTTGTGGATTC 3′ | (SEQ ID NO 105) |
| MB307 | |
| 5′ GAATCCACAAATTTCCTGACTAGACCA 3′ | (SEQ ID NO 106) |
| MB314 | |
| 5′ CTACTCATGGACTCCTGGCTTTG 3′ | (SEQ ID NO 107) |
| MB315 | |
| 5′ GCACAACAATTCCCATGTATTTAGCG 3′ | (SEQ ID NO 108) |
| MB316 | |
| 5′ CGCTAAATACATGGGAATTGTTGTGC 3′. | (SEQ ID NO 109) |

Sequencing reactions were composed of 4 μl of miniprep, 4 μl of primer (at 1 mM), 4 μl of water and 8 μl of BigDye Terminator Cycle Sequencing Ready Reaction (Applied Biosystems, Foster City, Calif.).

The PCR was carried out using Gene Amp PCR System 9700 (Applied Biosystems, Foster City, Calif.) using the PCR conditions in the instructions supplied with the Ready Reaction kit. Sequencing reactions were purified using the DyeEx Spin Kit (Qiagen, Valencia, Calif.) and dried for 20 minutes on low heat in a Speed Vac Plus (SC110A, from Savant, Holbrook, N.Y.) attached to a Universal Vacuum Sytem 400 (also from Savant). The reactions were resuspended in 4 μl of a 4 to 1 mixture of formamide (Sigma, St. Louis, Mo.) with 25 mM EDTA (Sigma) and 50 mg/ml dextran blue (Sigma). The reactions were then heated to 100° C. for 2 minutes and chilled on ice. The DNA was sequenced on an ABI 377 DNA Sequencer. The sequencing gel was prepared using a Long Ranger Singe1 Pack (BioWhittaker Molecular Applications, Rockland, Me.) according to the manufacturer's instructions. 2 μl of the sequencing reaction was loaded into each well of the gel. The gel was run for 3.5 hours using the 36E 2400 run module, the dye set DT (BD set Andy-Primer) and the dRHOD Matrix.

LXRLI1 clone sequences were compiled into contigs using either the small intestinal LXRLI1 clone sequences or the THP-1 LXRLI1 clone sequences as follows. One clone from both the small intestine and the THP-1 LXRLI1 clones were designated as a reference clone. The sequence of each reference LXRLI1 clone was first compared to published genomic clones from the AK000684 region (NCBI website, BlastN). When the sequence of the reference THP-1 or small intestinal LXRLI1 clone differed from the published sequence, the LXRLI1 sequence was then compared to the same region of sequence in the other sequenced clones from the same cell source to determine if the sequence difference observed was due to PCR error, or represented the consensus sequence for LXRLI1 in the small intestine or THP-1 cell line. Comparison of the consensus LXRLI1 nucleotide sequences obtained from the small intestine and THP-1 cells showed that the DNA sequence from small intestine differs by one base pair (from a G to a C at position 1435) from the sequence cloned from THP-1 cells. The final LXRLI1 cDNA sequence is set forth in SEQ ID NO 1. The predicted LXRLI1 protein from small intestine differs by one amino acid (from a Gly to an Ala at position 430) from the sequence predicted in THP-1 cells. The final LXRLI1 amino acid sequence is set forth in SEQ ID NO 2.

The nucleotide sequence of LXRLI1 cDNA (SEQ ID NO 1) was used to query the GenBank sequence database operated by the National Library of Medicine, in a BLAST (Basic Local Alignment Search Tool) search (Table 4). The search was performed using the following parameters: bl2seq, BLASTn program, non-default parameters—e 0.0001-FF. A BLAST search returns an Expect (E) value; the E value is the probability that a particular search result would have occurred by chance. Highly significant E values are greatly smaller than 1.0 (but larger than 0.0), while insignificant E values are close to 1.0.

The amino acid sequence of LXRLI1 protein (SEQ ID NO 2) was also used to query the GenBank sequence database (Table 5). The search was performed using the following parameters: bl2seq, BLASTp program, non-default parameters—e 0.0001-FF. * Amino acid sequences of the predicted protein products were compared to entries in two protein motif databases, Pfam and PROSITE. No significant protein motif matches were found. However, analyses of the LXRLI1 polypeptide sequence using two different membrane topology prediction programs, SOSUI (Hirokawa, et al., 1998 Bioinformatics 14:378-9) and TMpred (Hofmann and Stoffel, 1993 Biol. Chem. Hoppe-Seyler 374:166), suggests that the LXRLI1 protein has four (SOSUI) to seven (TMpred) transmembrane helices. These data suggest that LXRLI1 is a membrane protein.

SEQ ID NO 1 contained a full open reading frame that encoded a protein (SEQ ID NO 1) identical to the predicted FLJ22104 protein (SEQ ID NO 98), but contained an eighty-two additional amino acids at the amino terminus, an additional twenty-two amino acids, starting at amino acid 50 of SEQ ID NO 98) and an isoleucine rather than threonine at amino acid 235 of SEQ ID NO 98.

TABLE 4
BLASTn results for LXRLI1-encoding nucleotide sequences.

| | | | Maximum Percent | Maximum Percent | |
|---|---|---|---|---|---|
| | | Novel cDNA Blast | Identify over Sequence | Identify over Sequence | Maximum Length of |
| Novel cDNA | Polypeptide | Description | Window of 125 bp | Window of 275 bp | 100% Identity |
| SEQ ID NO 1 | SEQ ID NO 2 | 1. AK000684 | 1. 100% | 1. 100% | 1. 504 nucleotides |
| | | 2. AK013269 | 2. 96.8% | 2. 95.3% | 2. 68 nucleotides |
| | | 3. AK025757 | 3. 100% | 3. 100% | 3. 553 nucleotides |

TABLE 5
BLASTp results for LXARAI1 Protein.

| | | | | Prosite | Maximum Length of | Maximum Length of |
|---|---|---|---|---|---|---|
| Polypeptide | Blast Score | Blast Description | Pfam Motif(s) | Motif(s) | 100% Identity | 100% Similarity |
| SEQ ID NO 2 | 1. 870, E = 0.0 | 1. BAB28760 | 1. None | 1. None | 1. 112 amino acids out | 1. 146 amino acids out |
| | | [Mus musculus] | | | of a total of 458 | of a total of 458 |
| | 2. 711, E = 0.0 | 2. BAB15233 predicted | 2. None | 2. None | 2. 184 amino acids out | 2. 184 amino acids out |
| | | protein FLJ22104 | | | of a total of 458 | of a total of 458 |
| | | [Homo sapiens] | | | | |

### Example 3

Real Time Quantitative PCR of LXRLI1 Gene Expression

The relative expression of LXRLI1 mRNA was determined for a panel of human tissues using a quantitative real time polymerase chain reaction (PCR) based assay. Total RNA isolated from eight different human tissues was obtained from Ambion Inc. (Austin, Tex.). One hundred ng of total RNA from each tissue was subjected to a one-step reverse transcription-PCR amplification protocol following Applied Biosystems, Inc. (ABI, Foster City, Calif.) specifications as outlined in the ABI protocol manual 4310299B. The ABI human beta actin pre-developed assay reagent (PDAR #4326315E) was used to amplify the reference gene beta actin. The following LXRLI1 primer set: 57662FW 5′-GACGGTGGTACACTCTTGAGAAAA-3′ (SEQ ID NO 110) and 57662RV 5′-GAAGATGGGAAAACATTGTATAATTTAAGC-3′ (SEQ ID NO 111), designed using ABI primer express software, was used at a concentration of 300 nM. LXRLI1 primer optimization and validation of the 57662FW/57662RV primer set-target specificity was carried out prior to analysis of human tissue samples. Human beta actin amplification was detected using the actin specific 5′-VIC labeled TaqMan PDAR probes obtained from ABI. The LXRLI1 primer set described above was used to amplify a 119 base pair LXRLI1 specific amplicon that was detected by Syber Green binding and exhibited the predicted Tₘ of 74° C. All of the RT-PCT reactions were carried out in triplicate along with non-template controls using the ABI Prism 7000 sequence detection system. Critical threshold (C_{T}) values (Bustin, 2000 J. Mol. Endocrinol. 25:169-93) were obtained for each reaction, and average C_{T} values; were generated for each gene in each tissue as described in the TaqMan user manual (TaqMan Universal PCR Master Mix; Protocol, p.5; © 1998 PE Applied Biosystems).

The relative expression of LXRLI1 in each of eight tissues tested is depicted in Table 6 as a fold change in expression relative to LXRLI1 expression in kidney, as further normalized to beta-actin expression across all eight tissues. The data shown in Table 6 shows that LXRLI1 is expressed in all of the tissues examined. The highest relative level of LXELI1 expression was observed in adrenal and skeletal muscle tissue, with intermediate levels of expression in bladder, liver and testis tissue, and a low level of expression in lung, spleen and kidney tissue.

TABLE 6
Expression of LXRLI1 in Human Tissue (Fold expression relative to
expression level in Kidney as normalized with beta-actin expression level).

| Human Tissue | LXRLI1 |
|---|---|
| Adrenal | 23 |
| Skeletal Muscle | 20 |
| Bladder | 10.8 |
| Liver | 8.6 |
| Testis | 8.5 |
| Lung | 4.7 |
| Spleen | 1.4 |
| Kidney | 1.0 |

### Example 4

Use of LXR-Ligand Induced Markers to Classify LXR-Ligands

The 55 markers, representing the LXR-regulated geneset defined in Table 1, can also be used to classify LXR-ligands as exemplified in this example.

Microarray gene expression profiles were obtaining using RNA samples from different human cell lines exposed to LXR-agonist APD and to other treatments known to increase or decrease cellular cholesterol levels. Experimental methods were the same as those described in Example 1, except as described below.

22(R)-hydroxycholesterol (22RHC) is a LXR-ligand that is also thought to mimic cholesterol loading via suppression of SREBP processing (Brown and Goldstein, 1999 Proc. Natl. Acad. Sci. 96:11041-8). THP-1 cell samples were treated with 10 μM 22RHC (Sigma C 9384) in 10 mM ethanol. Cells were incubated in 22RHC for 3 hours, 6 hours and 20 hours, respectively, prior to extraction of total RNA.

Acetylated Low Density Lipoprotein (AcLDL) is a composition known to increase intracellular cholesterol levels, i.e., cholesterol loading (Chinetti, et al., 2001 Nature Med. 7:53-8; Fu, et al., 2001 J. Biol. Chem. 42:38378-87). THP-1 cells were cultured with AcLDL (200 μg/ml) for 3, 6, and 20 hours prior to extraction of total RNA.

Methyl-β-cyclodextrin (CD) is an inducer of cholesterol efflux from plasma membranes (Kilsdonk, et al., 1995, J. Biol. Chem. 270:17250-6). Medium A was aspirated from THP-1 cells and replaced with RPMI-1640 containing 2.0 mM methyl-β-cyclodextrin (Sigma C 4555), 0.1% BSA, and 100 nM PMA and the cells were incubated for 1 hour. This medium was then changed back to Medium A, and cells were collected for RNA measurements after 3 hours, 6 hours, and 20 hours of additional culture time.

Cholesterol mixed with CD results in the formation of water soluble cholesterol/CD complexes which are taken up by plasma membranes at an increased rate as compared to cholesterol alone (Christian, et al., 1997 J. Lipid Res. 38:2264-72). Cholesterol/CD mixture was prepared by dissolving 24.17 mg cholesterol powder in 5 ml of 100 mM CD (assume molecular weight 1338 for CD), shaking overnight at 37° C., and then filtering the cholesterol/CD complex solution through a 0.25 μm filter. The filtered cholesterol/CD complex was then added to THP-1 cells growing in Medium A to a final concentration of 0.5 mM CD, 24 μg/ml cholesterol. The THP-1 cells were cultured in Medium A with the CD and cholesterol mixture for 3, 6, and 20 hours prior to extraction of total RNA.

Gene expression profiles were obtained using the methods of Example 1, from twenty different RNA samples obtained from cell samples treated as described in the legend to FIG. 2. Expression levels for each of the markers comprising the LXR-regulated geneset were then sorted and rank ordered (X-axis) based upon their correlation to the ABCG1 marker expression profile across the twenty experimental conditions (Y-axis) represented in FIG. 2.

The results shown in FIG. 2 demonstrate that measurement of gene expression of the LXR-regulated geneset markers can be used to monitor cholesterol and lipid metabolism, and, to classify LXR ligand compounds. Inspection of the gene expression patterns displayed in FIG. 2 shows that the LXR-regulated geneset markers can be divided into two groups, designated “Group I” and “Group II” in FIG. 2. The Group I markers contain several genes, in particular, ABCA1 (SEQ ID NO 3), ABCG1 (SEQ ID NO 9), and SREBF1 (SEQ ID NO 5) that are well known to be directly regulated by LXR at the transcriptional level. In addition, Group I markers also contain a number of genes whose protein products are known to be directly involved in reverse cholesterol transport which serves to lower cholesterol and plant sterol absorption. In contrast, Group II markers include several genes, in particular, SCD (SEQ ID NO 7), FADS1 (SEQ ID NO 21) and FADS2 (SEQ ID NO 25) that are transcriptionally regulated by SREBF1. In addition, Group II markers are enriched for genes whose protein products are involved in lipid synthesis.

The data in FIG. 2 is also organized by experimental treatments on the Y-axis to show the differences in gene expression levels of the LXR-regulated geneset in response to cell treatments that are characterized as lowering cellular cholesterol levels verses those that cause an increase in cellular cholesterol levels. Inspection of the gene expression changes observed in the Group I markers as compared to the Group II markers across the cholesterol lowering and increasing treatments reveals that the Group I markers respond in a fashion that is reciprocal to the Group II response.

In summary, the data presented in FIG. 2 demonstrate the utility of monitoring the changes in gene expression levels of the LXR-regulated geneset markers set forth in Table 1. In particular, given the functional dichotomy between the markers in Group I verses Group II, the LXR-regulated geneset markers can be used to classify LXR-ligands into those that are general, i.e., affect markers in both groups, verses those that affect primarily Group I or Group II markers. The LXR-regulated geneset markers can also be used to determine the state of cholesterol metabolism in a cell sample by comparing the gene expression level of markers in Group I to the markers in Group II. Increased levels of gene expression of Group I markers and decreased levels of gene expression of Group II markers in a cell sample being indicative of a metabolic state of low cholesterol; while increased levels of gene expression of Group II markers and decreased levels of gene expression of Group I markers in a cell sample are indicative of a metabolic state of increased cholesterol. Based upon this result, the Group I and Group II markers can also be used in gene expression assays of corresponding transcript levels to classify LXR-ligands to determine whether the ligand compound affects expression of both Group I and Group II markers, or whether the ligand is selective in its affects on Group I or Group II markers.

All patents, patent publications, and other published references mentioned herein are hereby incorporated by reference in their entireties as if each had been individually and specifically incorporated by reference herein. While preferred illustrative embodiments of the present invention are shown and described, one skilled in the art will appreciate that the present invention can be practiced by other than the described embodiments, which are presented for,purposes of illustration only and not by way of limitation. Various modifications may be made to the embodiments described herein without departing from the spirit and scope of the present invention. The present invention is limited only by the claims that follow.

What is claimed:

1. A purified nucleic acid comprising SEQ ID NO 1, or the complement thereof.

2. The nucleic acid of claim 1, wherein said nucleic acid comprises a region encoding for the amino acid sequence of SEQ ID NO 2.

3. The nucleic acid of claim 1, wherein said nucleotide sequence encodes for a polypeptide consisting of the amino acid sequence of SEQ ID NO 2.

4. The nucleic acid of claim 1, wherein said nucleotide sequence comprises at least 554 consecutive nucleotides of SEQ ID NO 1, or the complement thereof.

5. A purified polypeptide comprising an amino acid sequence of SEQ ID NO 2.

6. The polypeptide of claim 5, wherein said polypeptide consists of amino acid sequence set forth in SEQ ID NO 2.

7. The polypeptide of claim 5, wherein said polypeptide comprises at least 185 consecutive amino acids of SEQ ID NO 2.

8. An expression vector comprising a nucleotide sequence encoding amino acid sequence set forth in SEQ ID NO 2, wherein said nucleotide sequence is transcriptionally coupled to an exogenous promoter.

9. The expression vector of claim 8, wherein said nucleotide sequence encodes for a polypeptide consisting of the amino acid sequence of SEQ ID NO 2.

10. The expression vector of claim 8, wherein said nucleotide sequence comprises SEQ ID NO 1.

11. The expression vector of claim 8, wherein said nucleotide sequence consists of the sequence of SEQ ID NO 1.

12. A recombinant cell comprising the expression vector of claim 8, wherein said cell comprises an RNA polymerase recognized by said promoter.

13. The recombinant cell of claim 12, wherein said cell is made by a process comprising the step of introducing the expression vector of claim 8 into said cell.

14. A method of estimating LXR activity in a subject, comprising:
(a) measuring a transcript level in a sample of mRNA or nucleic acid derived therefrom from said subject, wherein said transcript comprises a nucleotide sequence selected from the group consisting of SEQ ID NO 1, SEQ ID NO 13, SEQ ID NO 15, SEQ ID NO 17, SEQ ID NO 19, SEQ ID NO 21, SEQ ID NO 23, SEQ ID NO 25, SEQ ID NO 27, SEQ ID NO 29, SEQ ID NO 31, SEQ ID NO 33, SEQ ID NO 35, SEQ ID NO 37, SEQ ID NO 39, SEQ ID NO 41, SEQ ID NO 43, SEQ ID NO 45, SEQ ID NO 47, SEQ ID NO 49, SEQ ID NO 51, SEQ ID NO 53, SEQ ID NO 55, SEQ ID NO 57, SEQ ID NO 59, SEQ ID NO 61, SEQ ID NO 63, SEQ ID NO 65, SEQ ID NO 67, SEQ ID NO 69, SEQ ID NO 71, SEQ ID NO 73, SEQ ID NO 74, SEQ ID NO 76, SEQ ID NO 77, SEQ ID NO 79, SEQ ID NO 81, SEQ ID NO 82, SEQ ID NO 84, SEQ ID NO 85, SEQ ID NO 86, SEQ ID NO 87, SEQ ID NO 88, SEQ ID NO 89, SEQ ID NO 90, SEQ ID NO 91, SEQ ID NO 92, SEQ ID NO 93, SEQ ID NO 94 and SEQ ID NO 95; and
(b) comparing said measured level of said transcript to the level of said transcript measured in a control sample;
wherein the level of transcript measured in said sample from said subject as compared to the level of transcript measured in said control sample provides an estimate of LXR activity in said subject sample.

15. The method of claim 14 wherein said transcript level is measured using a method selected from the group consisting of a microarray, a Northern blot, and RT-PCR.

16. The method of claim 14 wherein said control sample is contacted with an LXR agonist.

17. The method of claim 16 wherein said comparing step is performed using a plurality of control samples, said plurality comprising at least one control sample not treated with an LXR agonist.

18. The method of claim 14 which is used to diagnose a disease or disorder involving LXR activity in a sample by detecting an increase or decrease in said transcript level relative to the amount present in an analogous sample from a subject not having the disease or disorder or not subjected to therapy.

19. The method of claim 18 wherein said disease or disorder is cholesterol gallstones, atherosclerosis, lipid storage diseases, obesity, diabetes, or hypercholesterolemia.

20. The method of claim 14 which is used to identify a compound that changes LXR activity, wherein said compound changes the estimated level of LXR activity in a sample from said subject contacted with said compound relative to the estimated level of LXR activity in an analogous sample from said subject not contacted with said compound.
